(19) United States
(12) Patent Application Publication
(10) Pub. No.: US 2018/0010091 A1
(43) Pub. Date: Jan. 11, 2018

(54) Hydrogel Comprising A Scaffold Macromer Crosslinked With A Peptide And A Recognition Motif

(71) Applicant: Massachusetts Institute of Technology, Cambridge, MA (US)

(72) Inventors: Linda G. Griffith, Cambridge, MA (US); Barbara Imperiali, Cambridge, MA (US); Christi D. Cook, Cambridge, MA (US); Caroline Chopko Ahrens, Lubbock, TX (US); Kasper Renggli, Cambridge, MA (US); Jorge L. Valdez Macias, Cambridge, MA (US)

(73) Assignee: Massachusetts Institute of Technology, Cambridge, MA (US)

(21) Appl. No.: 15/543,866

(22) PCT Filed: Jan. 15, 2016
(86) PCT No.: PCT/US2016/013495

§ 371 Date: Jul. 14, 2017

Related U.S. Application Data
(60) Provisional application No. 62/104,065, filed on Jan. 15, 2015.

(51) Int. Cl. C12N 5/00; C08G 65/333; C08J 3/24; C08G 65/334; C08J 3/075; G01N 33/574; G01N 33/50

(52) U.S. Cl. CPC C12N 5/0068; G01N 33/574; C08G 65/33396; G01N 33/5005; C08G 65/3342; C08J 3/075; C08J 3/24; C12N 5/0012; C12N 5/0062; C08G 2650/04; C08J 2371/02; C08G 2650/20; C08G 2210/00; C12N 2513/00; C12N 2533/30; C12N 2537/10

(57) ABSTRACT

Methods of forming, dissolving, and functionalizing an extracellular matrix gel on demand based on cross-linking, modification, and dissolution of hydrogels using transpeptidase (e.g. sortase) are disclosed. Also provided are hydrogels comprising one or more macromers crosslinked to a mixture of peptides, wherein all or a portion of the peptides in the mixture comprise a recognition motif cleavable by a transpeptidase (e.g., sortase).

### RELATED APPLICATION

This application claims the benefit of U.S. Provisional Application No. 62/104,065, filed on Jan. 15, 2015. The entire teachings of the above application are incorporated herein by reference.

### GOVERNMENT SUPPORT

The invention was made with Government support under Grant No. U54-CA112967, R01EB010246, and UH3TR000496 awarded by the National Institutes of Health, Grant No. CBET-0939511 awarded by the National Science Foundation, and W911NF-12-2-0039 awarded by the Defense Advanced Research Projects Agency. The government has certain rights in the invention.

### BACKGROUND OF THE INVENTION

Cell phenotypes, such as the malignant proliferation and invasion properties of carcinomas, are dramatically different when cells are cultured within three dimensional extracellular matrix (ECM) gels compared to culture on two dimensional substrates, and, in turn, are profoundly influenced by not only the composition of ECM, but also biophysical properties of the ECM, such as matrix stiffness and permeability (Griffith and Swartz, Nat. Rev. Mol. Cell Biol. 7, 211-224, 2006). Further, growing appreciation of the role of paracrine interactions between epithelia and stromal cells in tissue homeostasis and malignancy motivates development of new approaches to parse these interactions in vitro to understand dynamic cell-cell communication and signaling (Rothberg et al., Biochimica et Biophysica Acta (BBA)—Proteins and Proteomics 1824, 123-132, 2012). These concerns are driving efforts to capture complex human physiology in culture with new biomaterials and devices that foster formation of 3D cells, tissues, and organ subsystems, along with new approaches to measure and interpret dynamic extracellular and intracellular signaling networks within such complex models. Currently available systems limit the ability to measure and interpret such signaling networks accurately for various reasons.

Natural ECM gels, particularly collagen and Matrigel®, have demonstrated great utility in eliciting relevant biological behaviors in vitro and they remain workhorses for the general cell biology community as they are readily available. However, the biophysical and compositional properties of native ECM are difficult to tune in modular fashion, and dissolution of these gels to release cells for further analysis requires long incubations in protease solutions, if it can be accomplished at all. Long incubation times are undesirable because, for example, intracellular signaling networks are highly dynamic and respond within minutes to changes in external cues. Moreover, the use of proteases to break down the 3D tissue ECM alters the very properties under investigation in models of physiological processes such as 3D malignant invasion. Furthermore, although a spectrum of synthetic and semi-synthetic hydrogels enabling modular control of cell adhesion, degradation, stiffness, and other properties have been described, broad adoption of these is limited by gaps in functionality as well as accessibility.

While gels, including hydrogels, represent advantageous systems due to their physical properties, optimal strategies to improve and modify them with biological cues are needed.

### SUMMARY OF THE INVENTION

Many approaches, such as thermal, chemical, and ionic shifts have been deployed to release cells, as has photodegradation, but these approaches are relatively slow, have variable success in minimizing cell damage, or are limited in application to relatively thin tissues. Further, many strategies have been explored to functionalize hydrogels with ECM-derived biological cues such as adhesion peptides and growth factors. For example, photopolymerization is a useful and commonly used technique. However, the use of UV radiation may result in damage to the cells encapsulated in the hydrogels. Other chemical approaches are available (e.g., NHS chemistry and copper-catalyzed alkyne-azide cyclo-addition), but many of these approaches are toxic, technically challenging, or may result in undesired damage or alteration to the protein or peptide used to functionalize the hydrogels.

The present invention addresses the substantial need in creating gels that can be formed and dissolved on demand using methods accessible to the general cell biology community. In some embodiments, the present method also allows for rapid dissolution of gels and release of cells (or other biomaterial encapsulated within the gels), allowing for accurate analysis and interpretation of cellular events (e.g., signaling). In some embodiments, the present method also enables partial dissolution of gels to, for example, modify the properties of the gel (e.g., physical properties such as density and stiffness). In some embodiments, the present method also provides a method to readily incorporate, with high specificity, relatively large peptides and proteins in gels with high yield and to specifically remove or exchange these functional groups. In particular, the present invention provides methods of transpeptidase (e.g., sortase) mediated formation, dissolution, and/or functionalization of crosslinked gels. The present invention provides methods that allow formation and dissolution of gels rapidly on demand with potentially very little impact on cells.

Accordingly, in one embodiment, the present invention provides a hydrogel comprising one or more scaffold macromers crosslinked to a mixture of peptides, wherein all or a portion of the peptides in the mixture comprise a recognition motif cleavable by a transpeptidase. In a related embodiment, the present invention also provides a method of dissolving the hydrogel, said method comprising treating the hydrogel with a first transpeptidase and a peptide comprising an acceptor substrate sequence of the first transpeptidase under conditions that promote dissolution of the hydrogel, thereby dissolving the hydrogel.

In another embodiment, the present invention provides a method of forming a hydrogel dissolvable by a transpeptidase, said method comprising: combining 1) a mixture of peptides, wherein all or a portion of the peptides in the mixture comprise a recognition motif cleavable by a transpeptidase, each peptide having a first crosslinking moiety; 2) one or more scaffold macromers having a second crosslinking moiety; and 3) a suitable crosslinking agent under suitable conditions that promote crosslinking of the first and second crosslinking moieties, thereby forming a hydrogel dissolvable by a transpeptidase.

In other embodiments, the present invention provides a method of dissolving a hydrogel, said method comprising: treating a hydrogel comprising a transpeptidase recognition motif with a transpeptidase and a peptide comprising an acceptor substrate sequence under conditions that promote dissolution of the hydrogel, thereby dissolving the hydrogel.

In another embodiment, the present invention provides a method of dissolving a hydrogel, said method comprising: treating a hydrogel comprising a sortase recognition motif with a sortase and a peptide comprising an acceptor substrate sequence under conditions that promote dissolution of the hydrogel, thereby dissolving the hydrogel.

In another embodiment, the present invention provides methods of forming a hydrogel that comprises a pendant transpeptidase recognition motif, said method comprising: combining one or more scaffold macromers having a first crosslinking moiety, a transpeptidase recognition motif having a second crosslinking moiety at its N-terminal end, and a suitable crosslinking agent under conditions that promote crosslinking of the first and second crosslinking moieties, thereby forming a hydrogel that comprises a pendant transpeptidase substrate sequence. In certain embodiments, the transpeptidase recognition motif further comprises a biomolecule at its C-terminal end.

In a further embodiment, the present invention provides methods of forming a functionalized hydrogel, said method comprising: combining a first scaffold macromer having a terminal first transpeptidase recognition motif; a second scaffold macromer having a terminal transpeptidase acceptor substrate sequence; one or more biomolecules having a terminal transpeptidase recognition motif or an acceptor substrate sequence; and a transpeptidase under conditions that promote transpeptidase ligation of the transpeptidase recognition motif with the acceptor substrate sequence, thereby forming a functionalized hydrogel.

In another embodiment, the present invention provides a kit for hydrogel formation comprising: an isolated transpeptidase enzyme; and a plurality of scaffold macromers, wherein said plurality comprises at least a first macromer having a terminal transpeptidase recognition motif, and at least a second macromer having a terminal transpeptidase acceptor substrate sequence.

BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

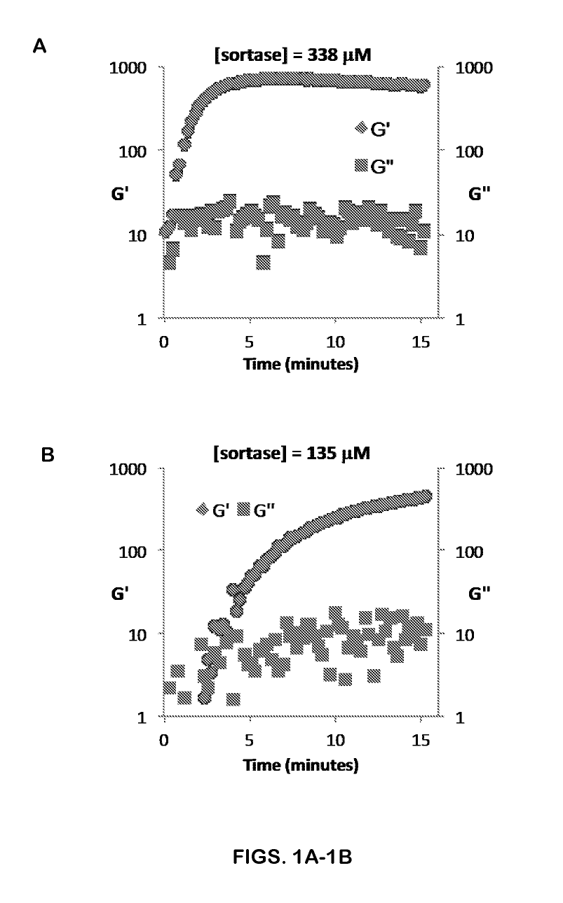

FIGS. 1A and 1B illustrate the time-dependent storage (G′) and loss (G″) moduli (Pa) for Sortase A (SrtA)-3M mediated gel formation at 338 μM sortase (FIG. 1A) and 135 μM sortase (FIG. 1B).

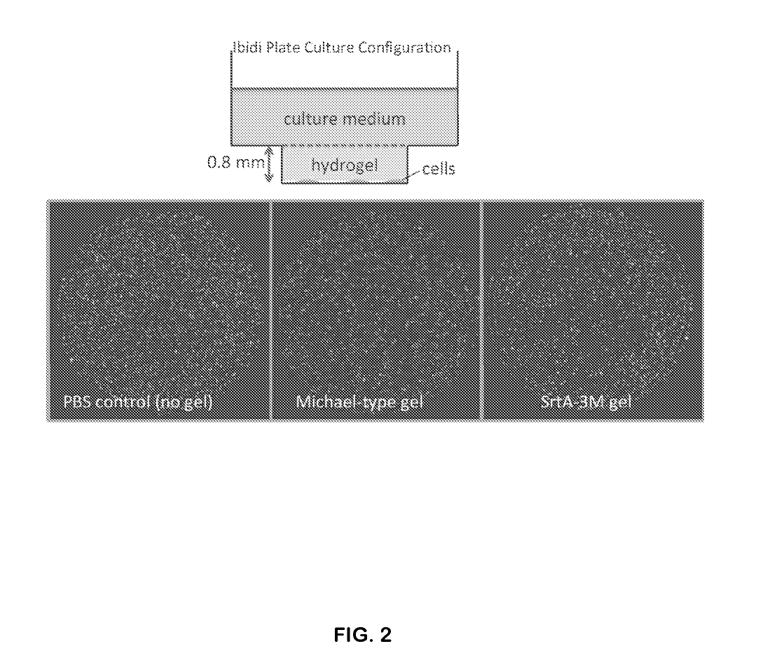

FIG. 2 depicts culture configuration (top panel) and live/dead viability stains for MSC cultured as indicated (bottom panel). SrtA-3M crosslinking concentration was 338 μM. Green=viable, red=dead.

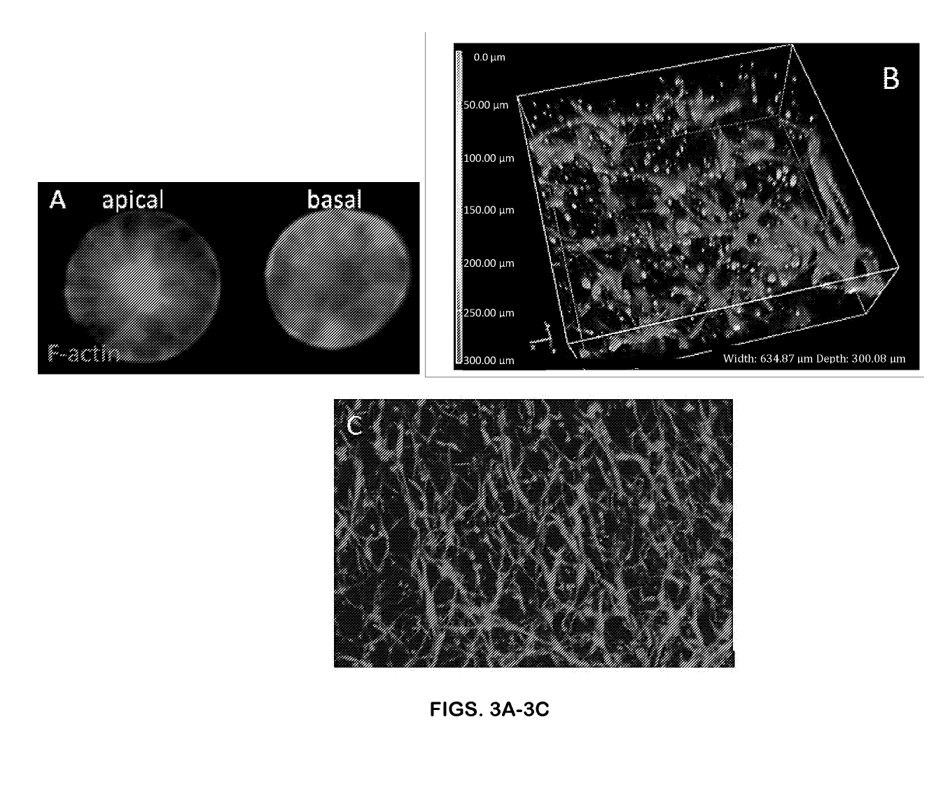

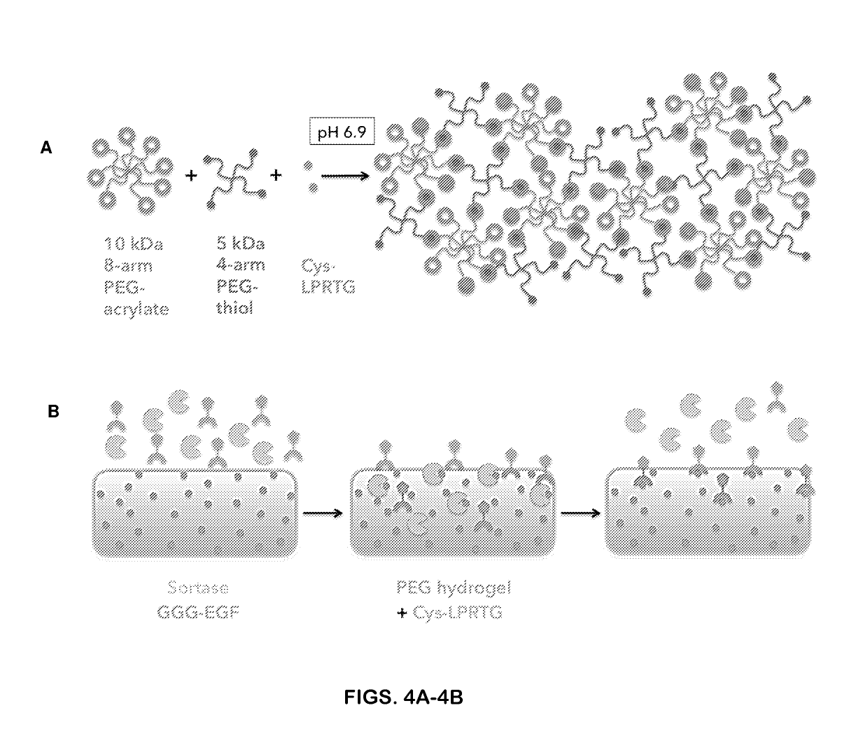

FIGS. 3A-3C illustrate functionalized PEG hydrogel morphogenesis assays. FIG. 3A shows polarization of endometrial epithelia as a function of adhesion ligand composition. FIG. 3B depicts tube formation by iPS-derived endothelial cells, and FIG. 3C shows network formation by mesenchymal stem cells (MSC).

FIGS. 4A and 4B show schematics of hydrogel formation (FIG. 4A) and sortase-mediated GGG-EGF tethering (FIG. 4B). FIG. 4A shows gel formation through Michael-type addition.

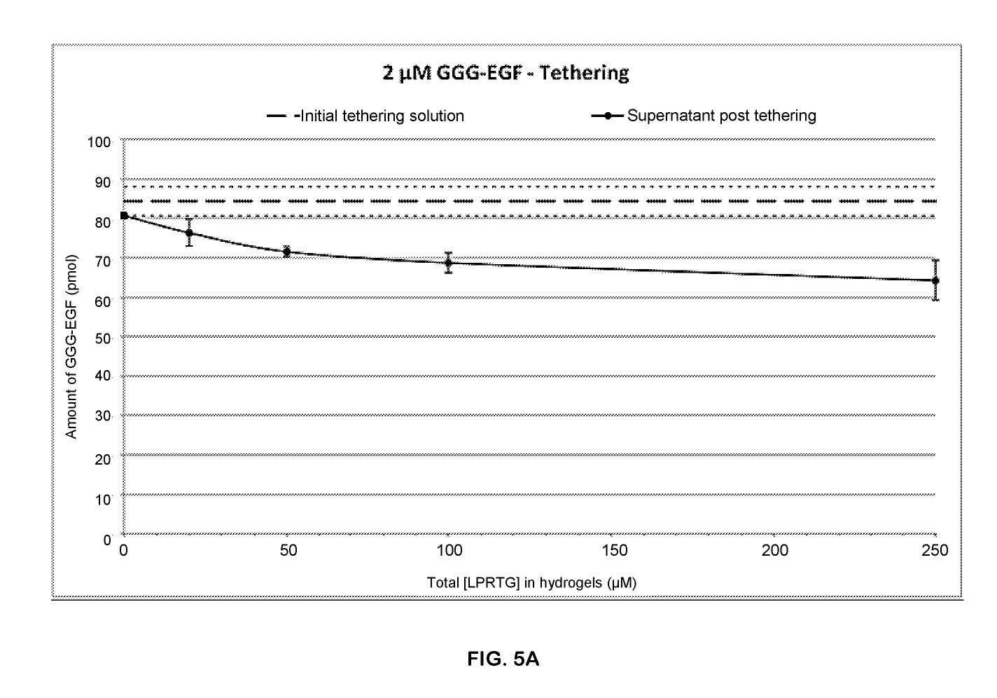

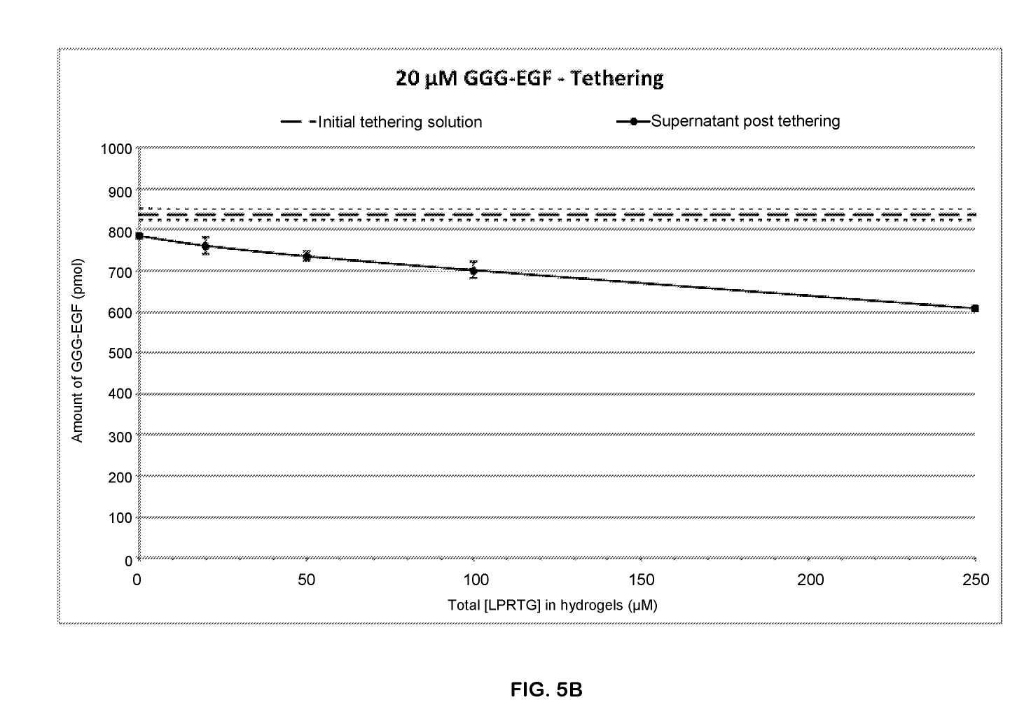

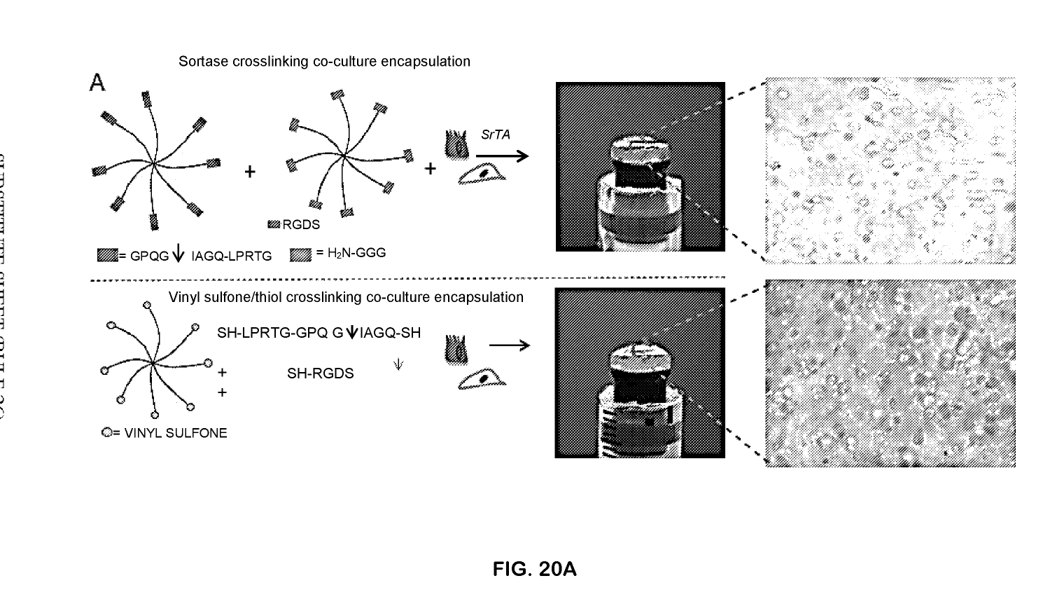

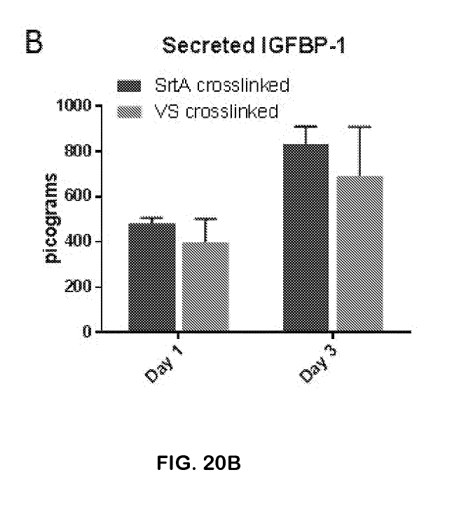

FIGS. 5A and 5B depict tethering sandwich ELISA at 2 μM GGG-EGF (FIG. 5A) and 20 μM GGG-EGF (FIG. 5B).

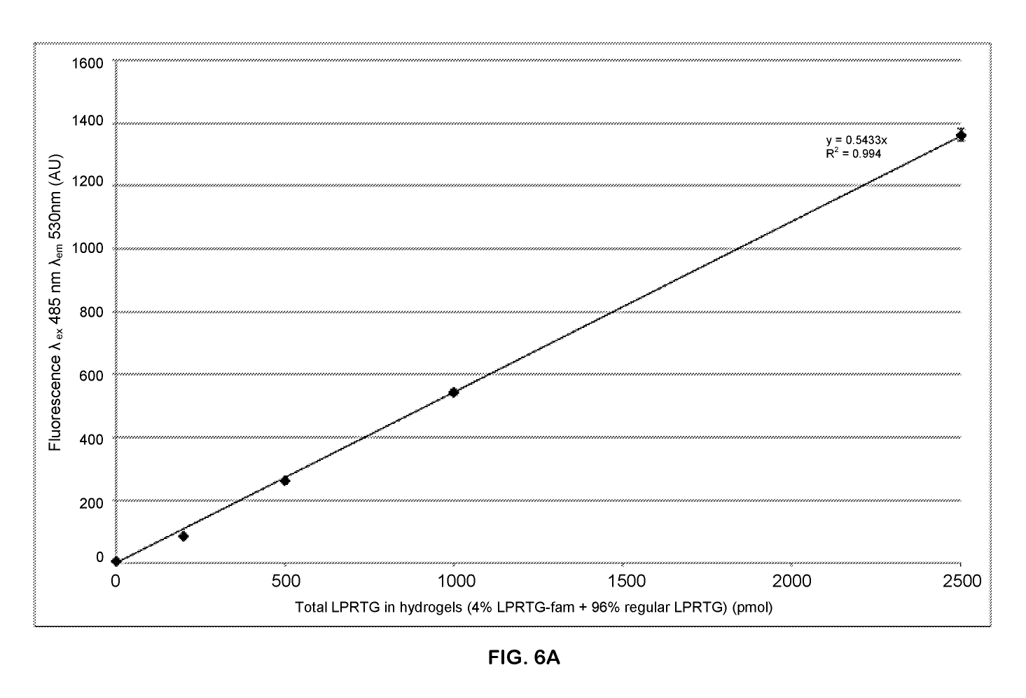

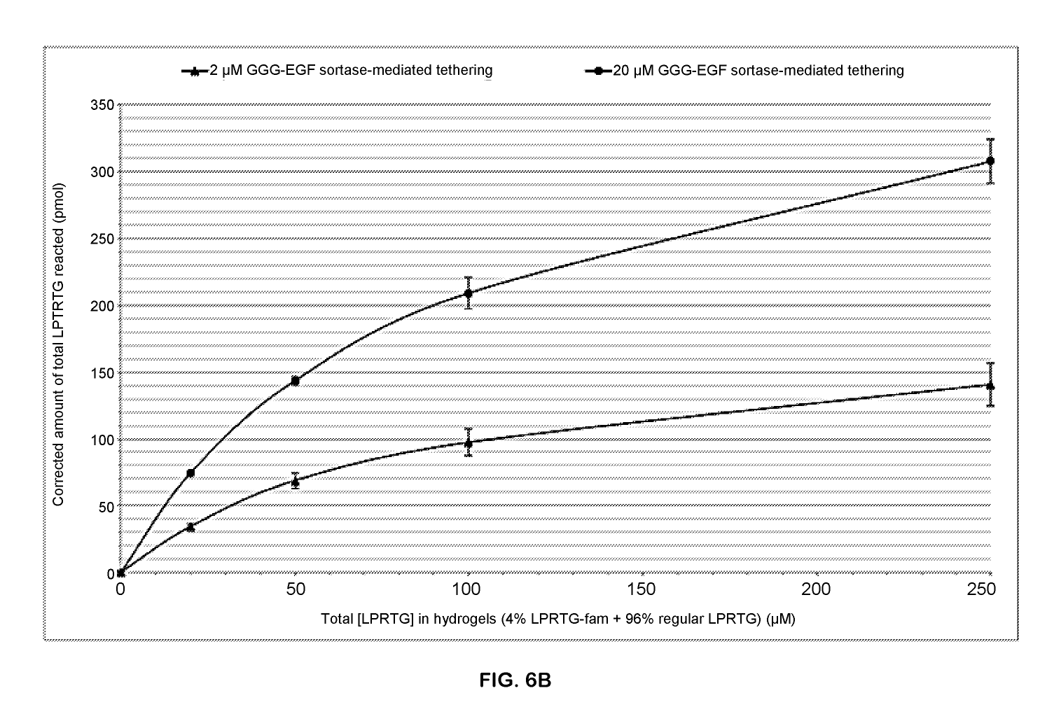

FIGS. 6A and 6B show fluorescence measurements in the tethering assays. FIG. 6A shows a linear standard curve generated by measuring fluorescence of hydrogels containing 0, 20, 50, 100, or 250 μM of total LPRTG peptide. FIG. 6B shows the corrected amount of reacted LPRTG with increasing LPRTG concentration in hydrogels, measured for both 2 μM and 20 μM GGG-EGF. The y-axis of FIG. 6A reads “Fluorescence λₑₓ, 485 nm λₑₘ 530 nm (AU); the upper right hand corner of FIG. 6A reads “y=0.5433x, R²=0.9994”.

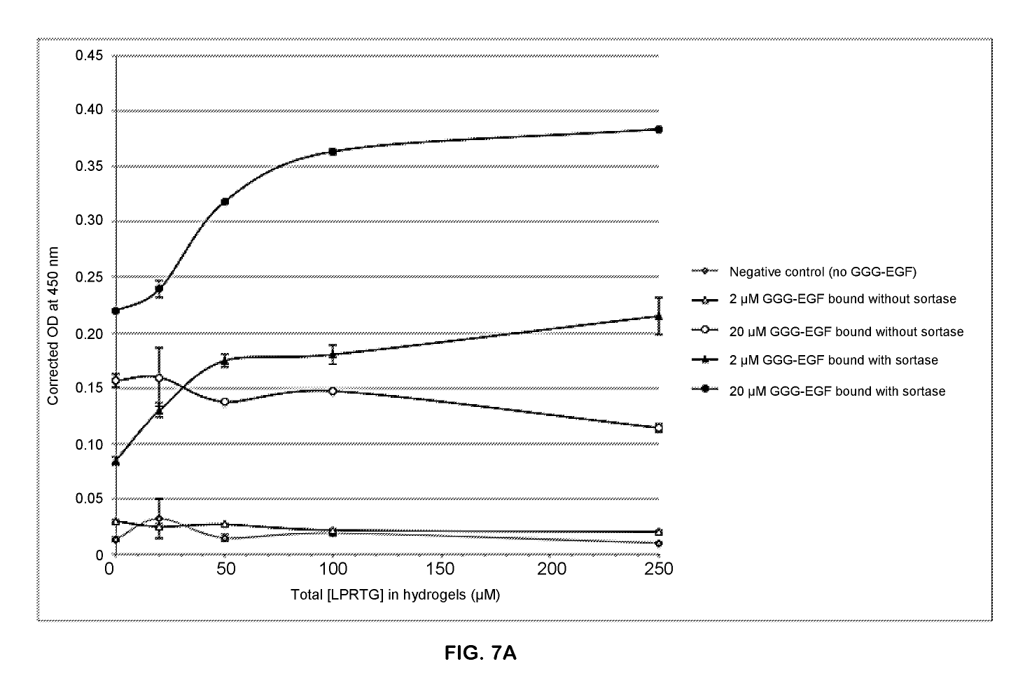

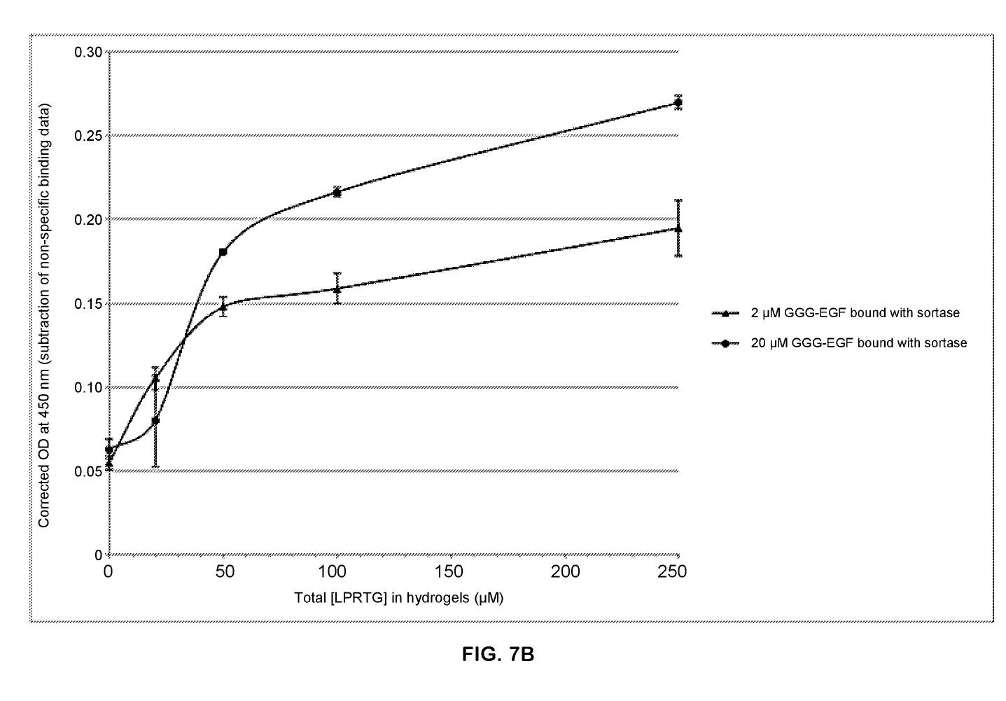

FIGS. 7A and 7B depict direct ELISA on hydrogels detecting the presence of EGF. FIG. 7A shows the degree of non-specific binding of GGG-EGF in the absence of SrtA; and FIG. 7B shows a corrected direct ELISA, to directly compare SrtA-mediated tethering at 2 and 20 mM GGG-EGF.

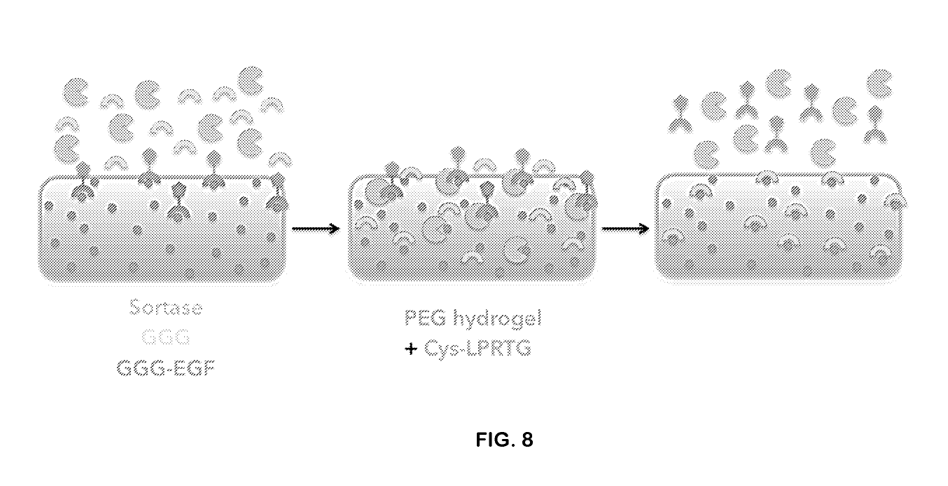

FIG. 8 illustrates a schematic of tethered GGG-EGF cleavage by SrtA.

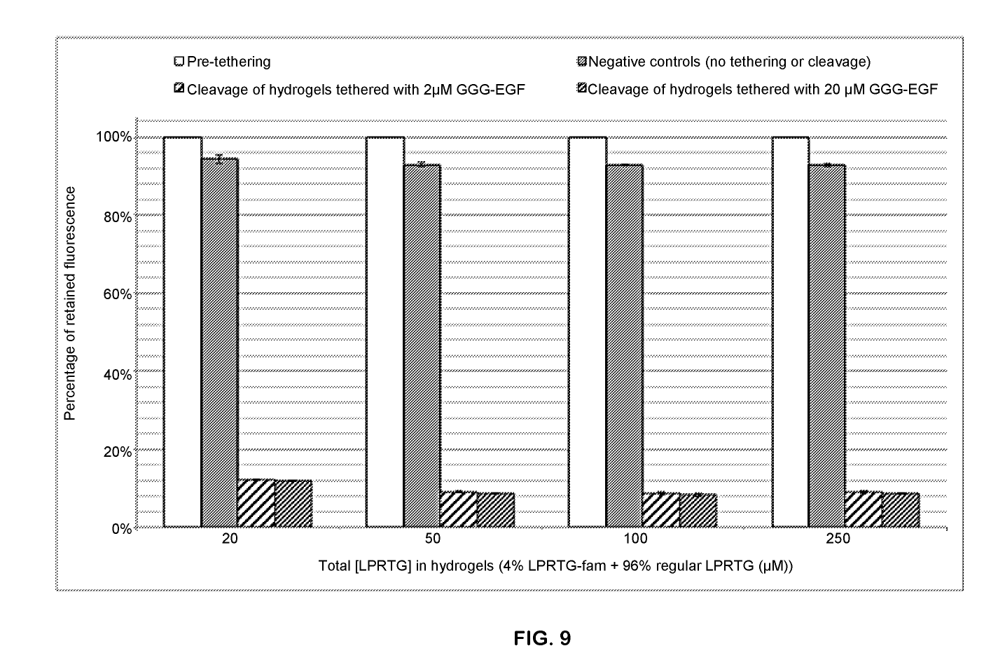

FIG. 9 depicts fluorescence measurements before and after SrtA-mediated cleavage of tethered GGG-EGF (tethered with 2 or 20 μM GGG-EGF), in various hydrogel concentrations of LPTRG.

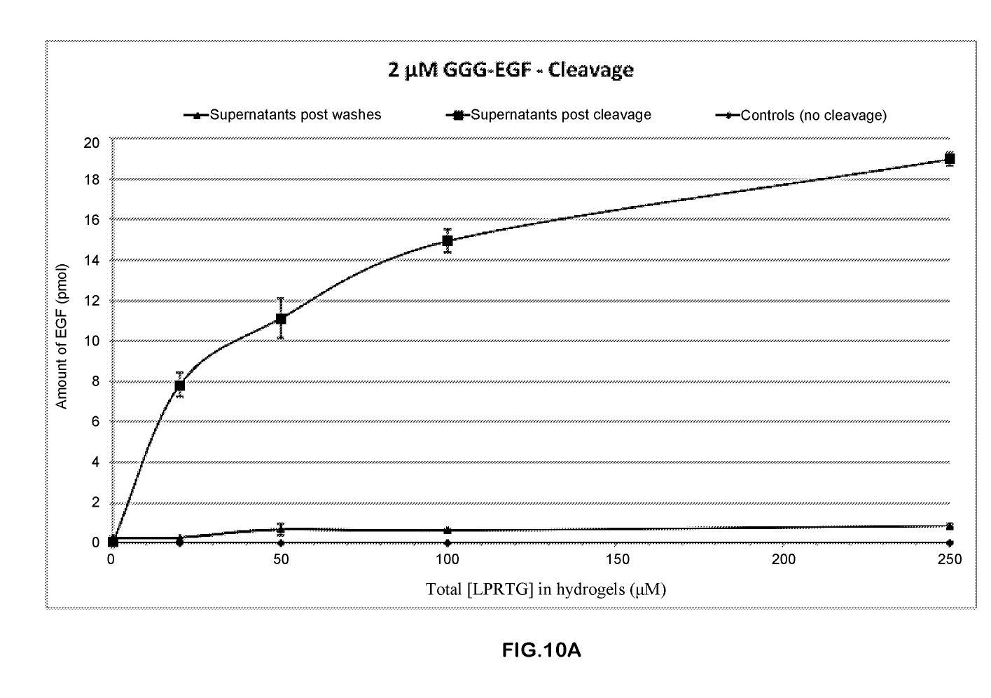

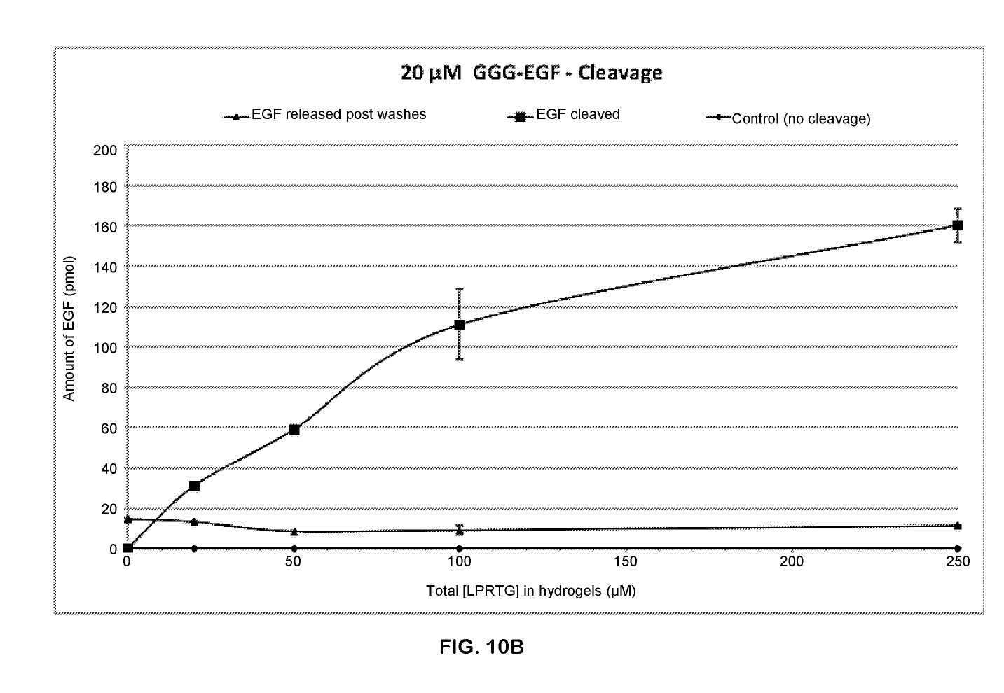

FIGS. 10A and 10B depict the amount of released GGG-EGF as a function of LPRTG concentration for tethering at 2 μM (FIG. 10A) and 20 μM (FIG. 10B).

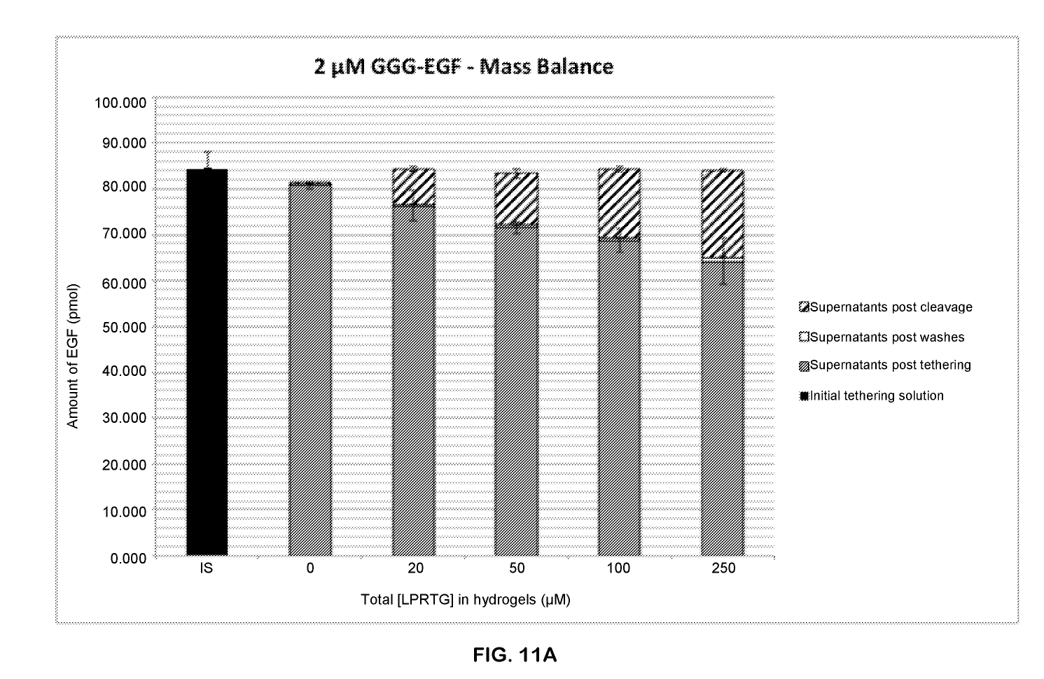

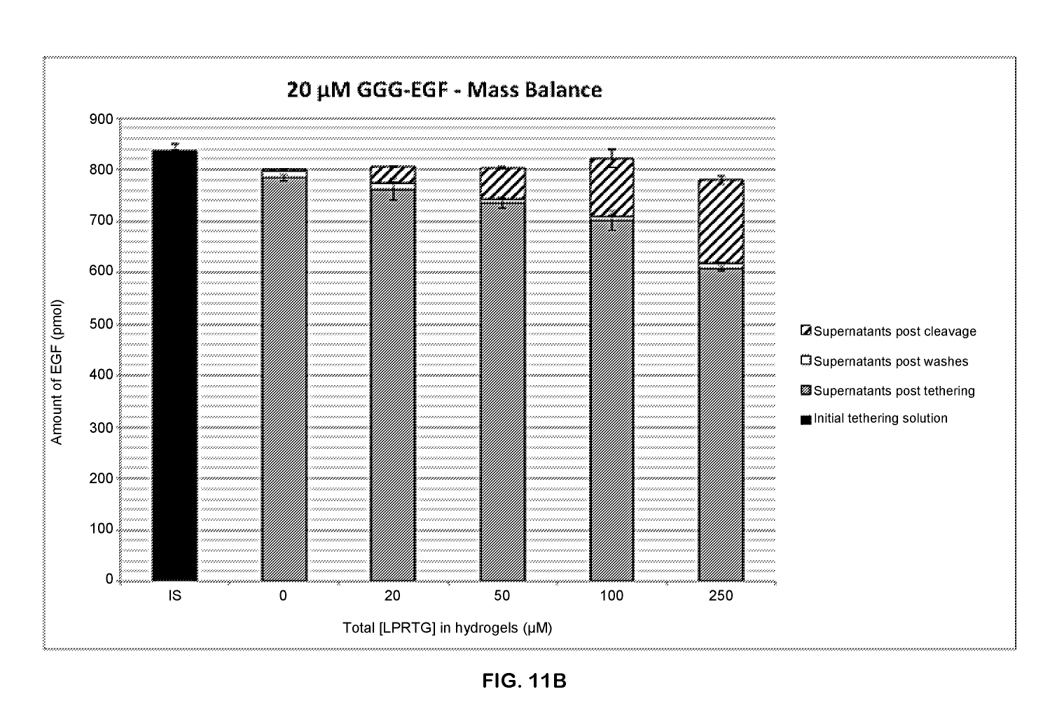

FIGS. 11A and 11B illustrate the mass balance achieved, showing that the sum of the amount of GGG-EGF remaining in solution after tethering, the amount of GGG-EGF released by washes, and the amount of cleaved GGG-EGF matches the amount of EGF in the initial tethering, when tethered at 2 μM (FIG. 11A) or 20 μM (FIG. 11B) GGG-EGF.

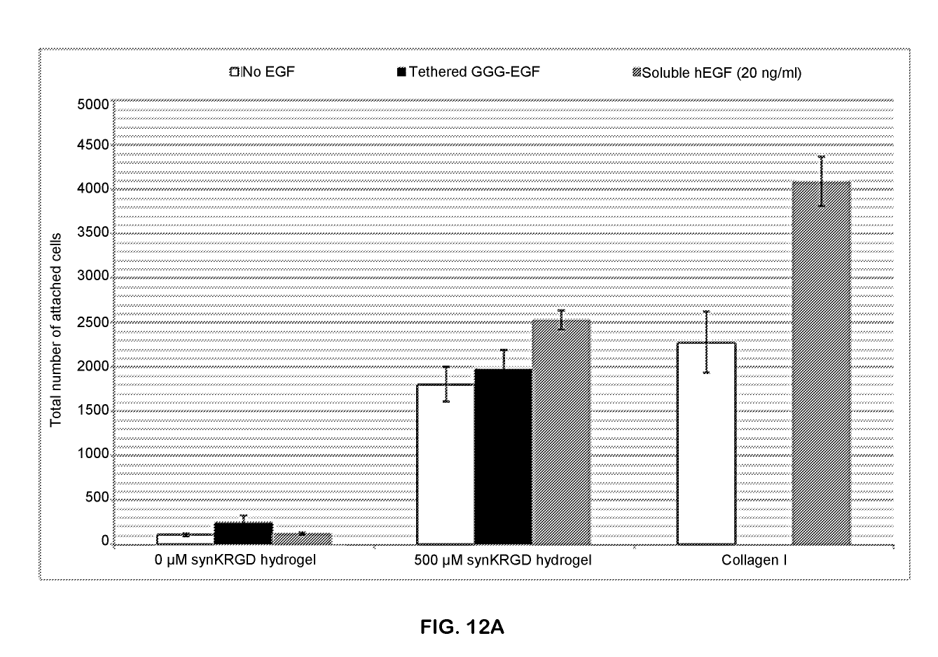

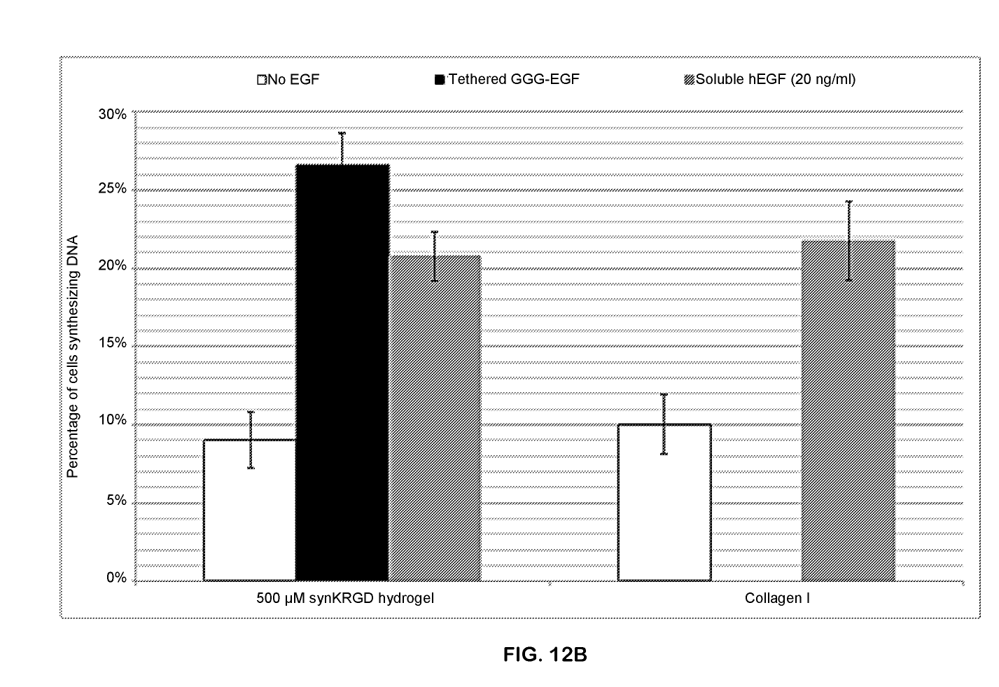

FIGS. 12A and 12B show results of cell attachment (FIG. 12A) and DNA synthesis assay (FIG. 12B) of hepatocytes.

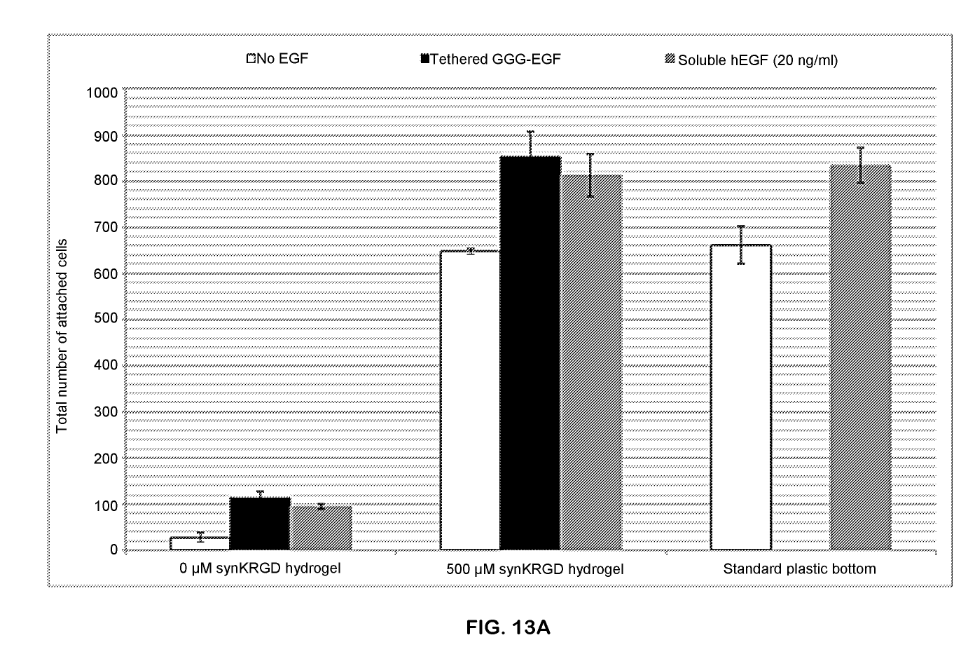

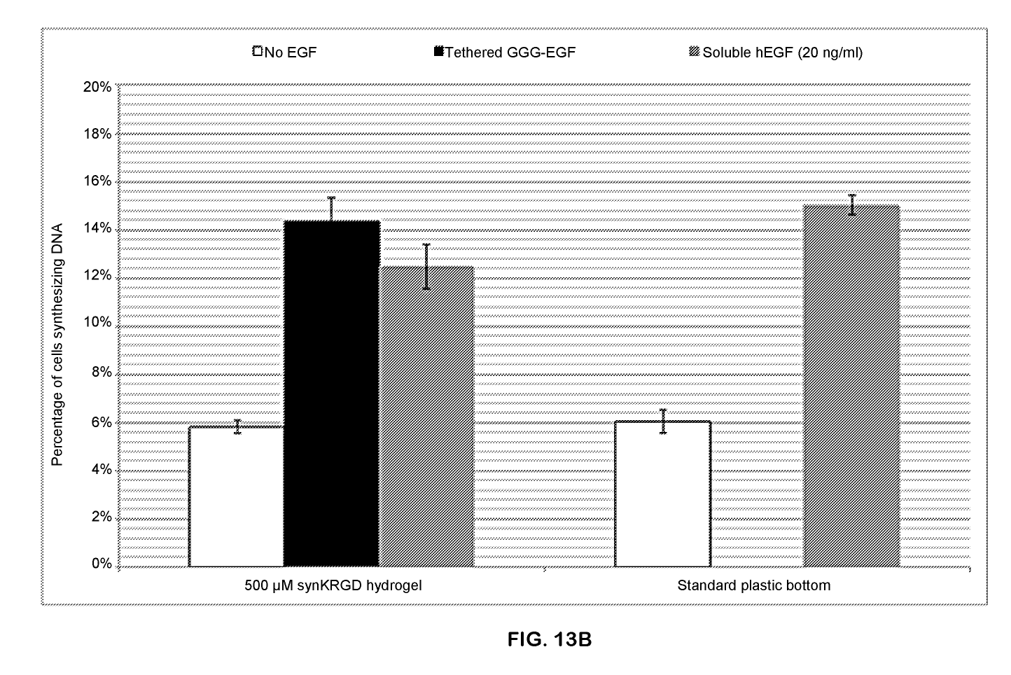

FIGS. 13A and 13B show results of cell attachment (FIG. 13A) and DNA synthesis assay (FIG. 13B) of endometrial epithelial cells.

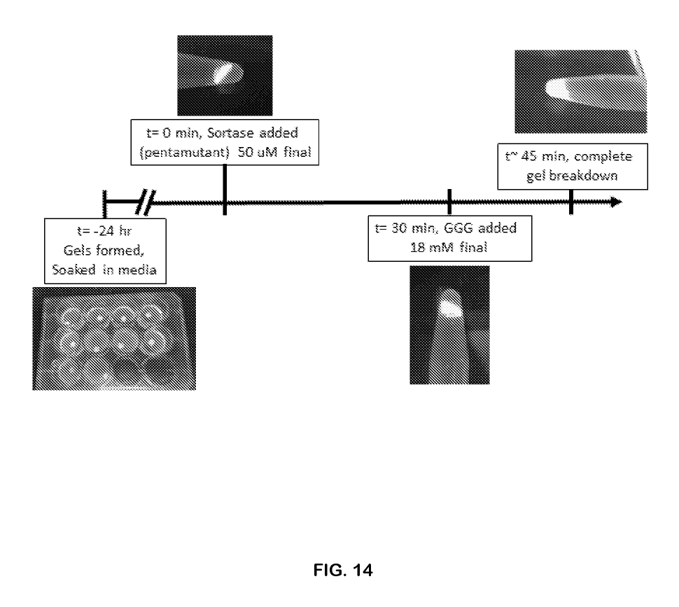

FIG. 14 illustrates visualization of sortase-mediated hydrogel dissolution in the presence of GGG over a time line.

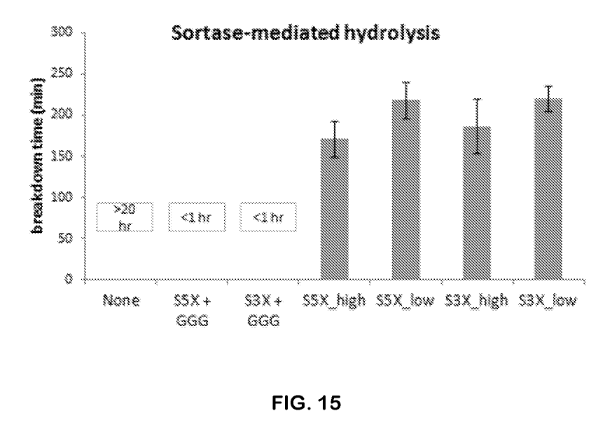

FIG. 15 shows a comparison of sortase-mediated gel degradation in the presence and absence of GGG in preliminary experiments; “high” indicates 416 μM and “low” indicates 250 μM of sortase. “S5X” refers to SrtA-5M pentamutant and “S3X” refers to SrtA-3M triple mutant.

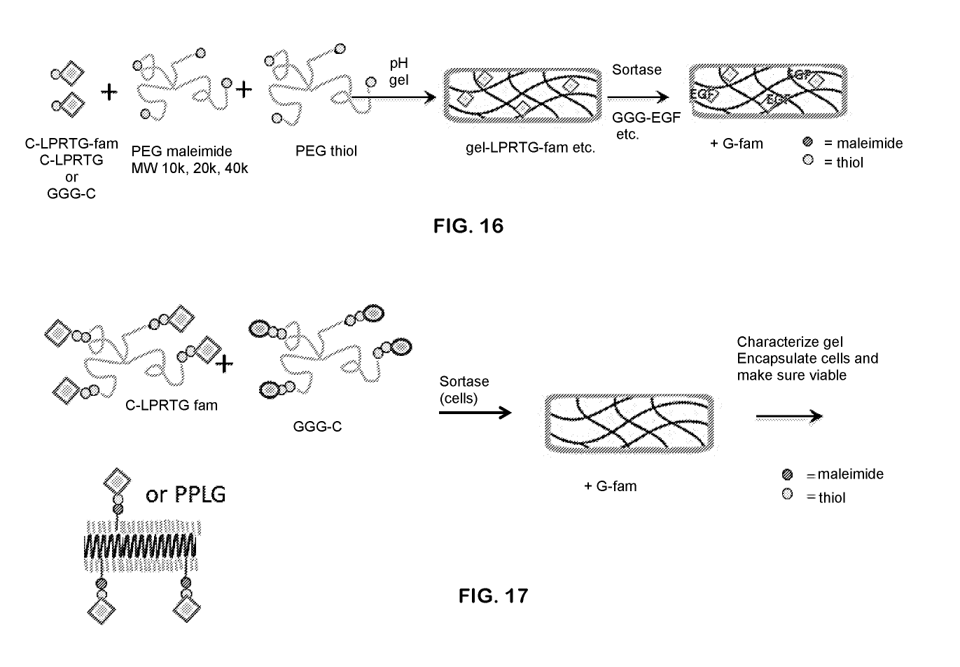

FIG. 16 shows a generalized schematic of sortase grafting EGF or Neuregulin to PEG hydrogels. Boxes represent C-LPRTG-fam, C-LPRTG, or GGG-C. Green dots represent maleimide groups; blue dots represent thiol groups. Gray lines represent PEG.

FIG. 17 shows a schematic of sortase-mediated bulk crosslinking to form PEG hydrogels. Green boxes represent C-LPRTG-fam, and blue circles represent GGG-C. Green dots represent maleimide groups; blue dots represent thiol groups; gray lines represent PEG.

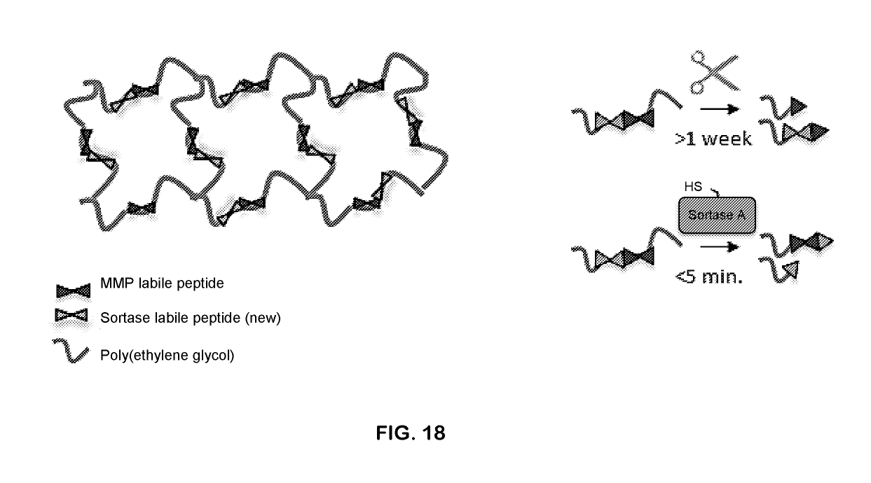

FIG. 18 illustrates formation of a hydrogel that contains a sortase substrate sequence (“sortase labile peptide” or “sortase sensitive peptide”) and a substrate sequence cleaved by matrix metalloproteases (MMP).

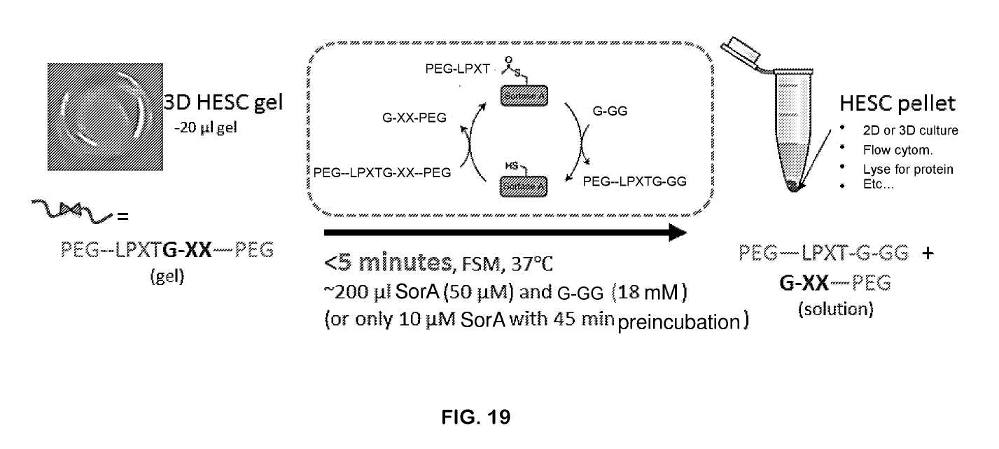

FIG. 19 shows a schematic of a reaction mediated by SrtA to dissolve gels rapidly. “FSM” is full serum media; “SorA” refers to “SrtA”.

FIGS. 20A-20B show a comparison of cell encapsulation methods using hydrogel formed by sortase crosslinking and vinyl sulfone/thiol crosslinking. FIG. 20A (top panel) shows gel encapsulation strategy using SrtA to catalyze crosslinking and incorporation of the adhesive motif RGD. FIG. 20A (bottom panel) shows gel encapsulation strategy using vinyl sulfone (VS)/thiol chemistry to crosslink and incorporate the adhesive motif RGD. FIG. 20B shows cumulative IGFBP-lsecreted to culture media in endometrial stromal/epithelial co-cultures by days 1 and 3 from hydrogels crosslinked either through SrtA catalysis or VS/thiol chemistry.

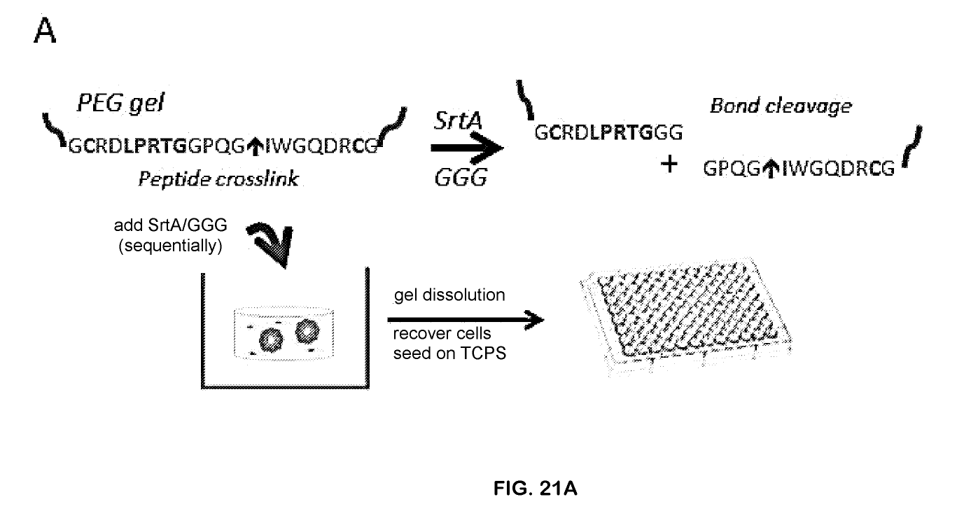

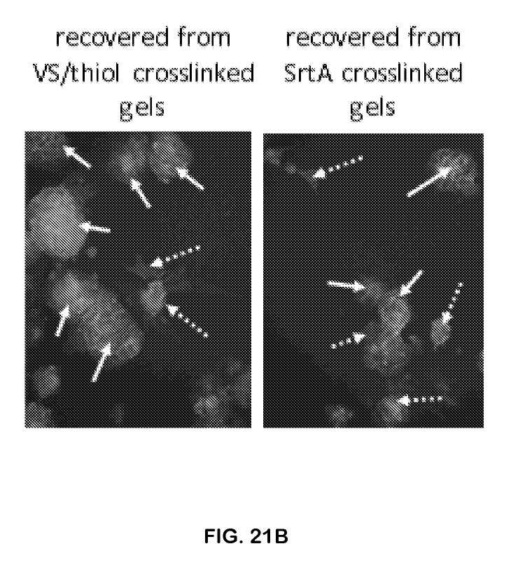

FIGS. 21A-21B depict endometrial stromal/epithelial cell release from PEG hydrogel through sortase-mediated dissolution. FIG. 21A shows gel dissolution strategy using SrtA and soluble GGG and recovery of cells from 3D hydrogels. FIG. 21B shows cells recovered from 3D co-cultures at day 6 and seeded on TCPS. Example of Ber-EP4 staining indicated by thick arrow; example of CD10 staining indicated by dashed arrow.

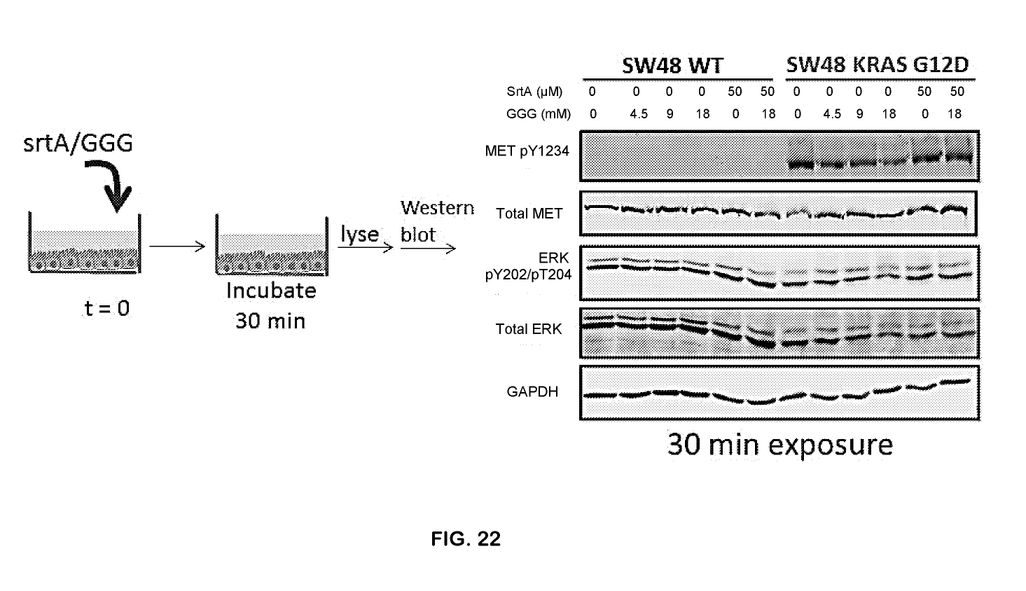

FIG. 22 shows the effect of srtA and GGG combinations on phosphorylation activity of ERK and MET.

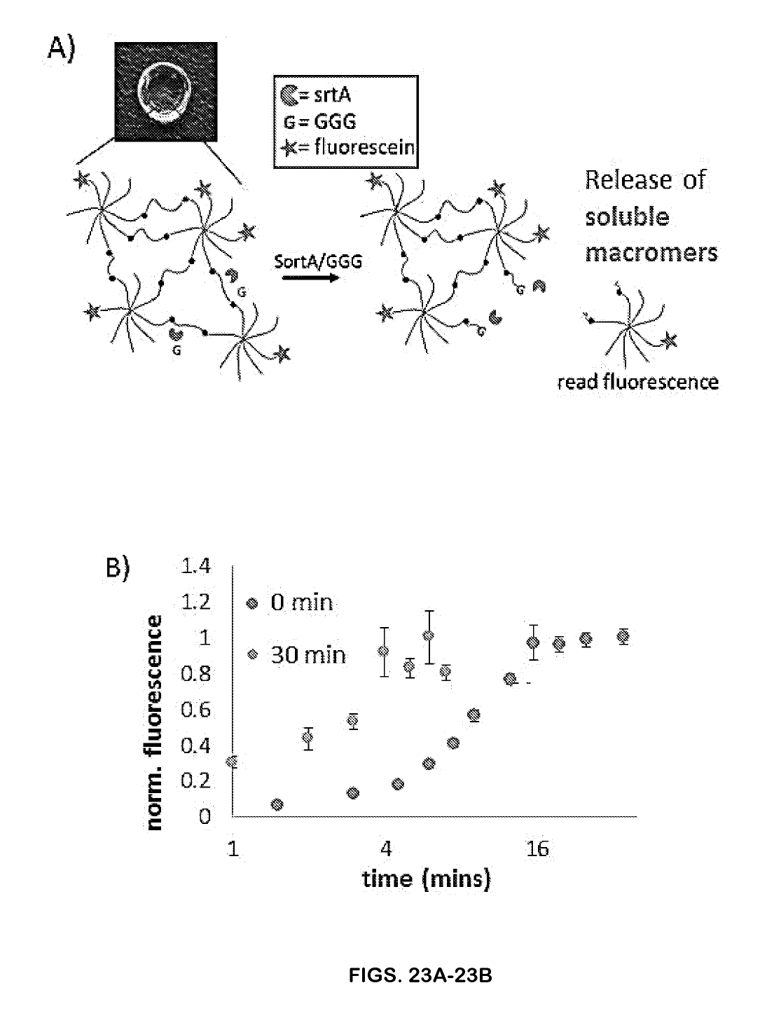

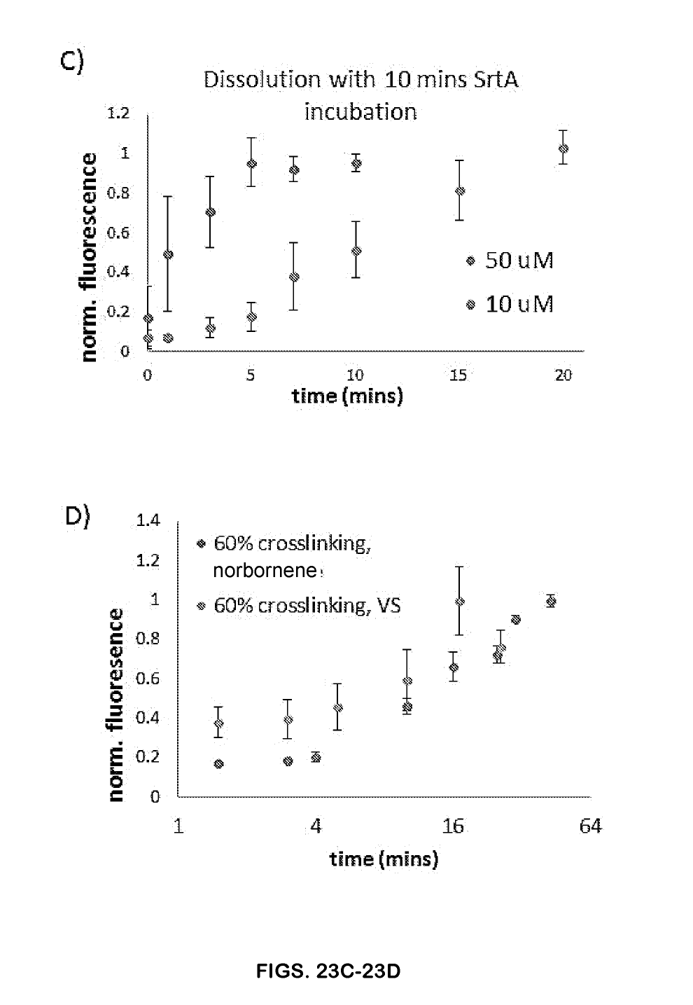

FIGS. 23A-23D show the results of SrtA-mediated hydrogel dissolution kinetics studies. FIG. 23A illustrates the assay quantification scheme. FIG. 23B depicts hydrogel dissolution with 30 mins incubation of srtA before addition of GGG and with no incubation before addition of GGG. FIG. 23C shows hydrogel dissolution after 10 min incubation with SrtA at 10 uM and 50 uM. FIG. 23D shows SrtA-mediated dissolution of hydrogels synthesized via norbornene-thiolene or Michael-type (vinyl sulfone) crosslinking chemistry.

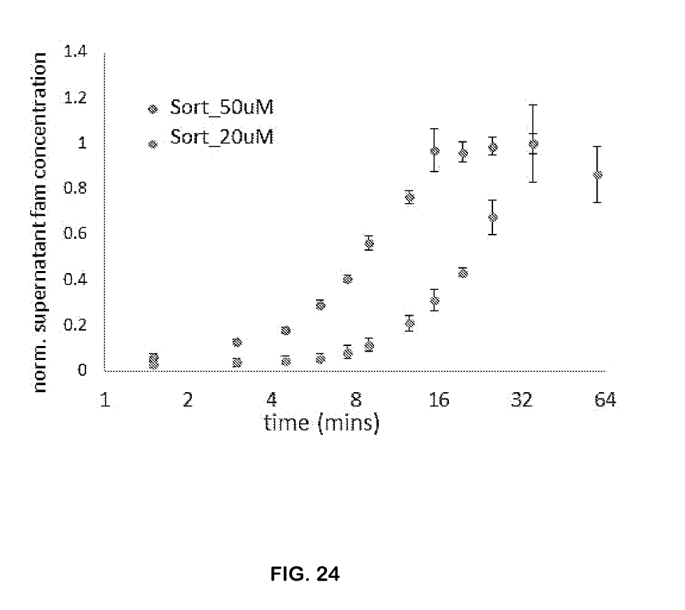

FIG. 24 demonstrates that dissolution time is tunable through sortase concentration.

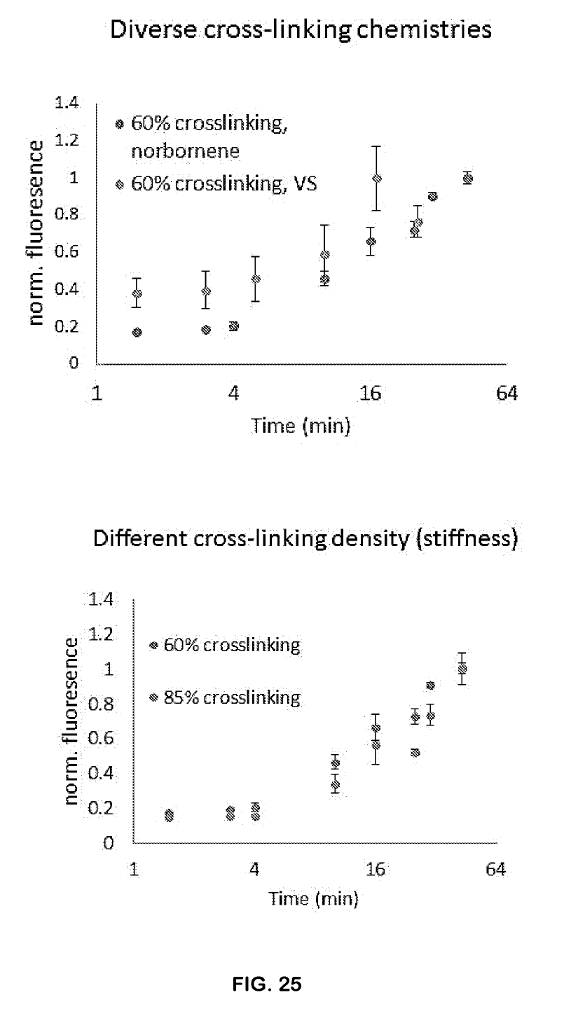

FIG. 25 shows that sortase-mediated dissolution is robust to different types of gels. Here, 50 uM sortase and 18 mM GGG were added simultaneously.

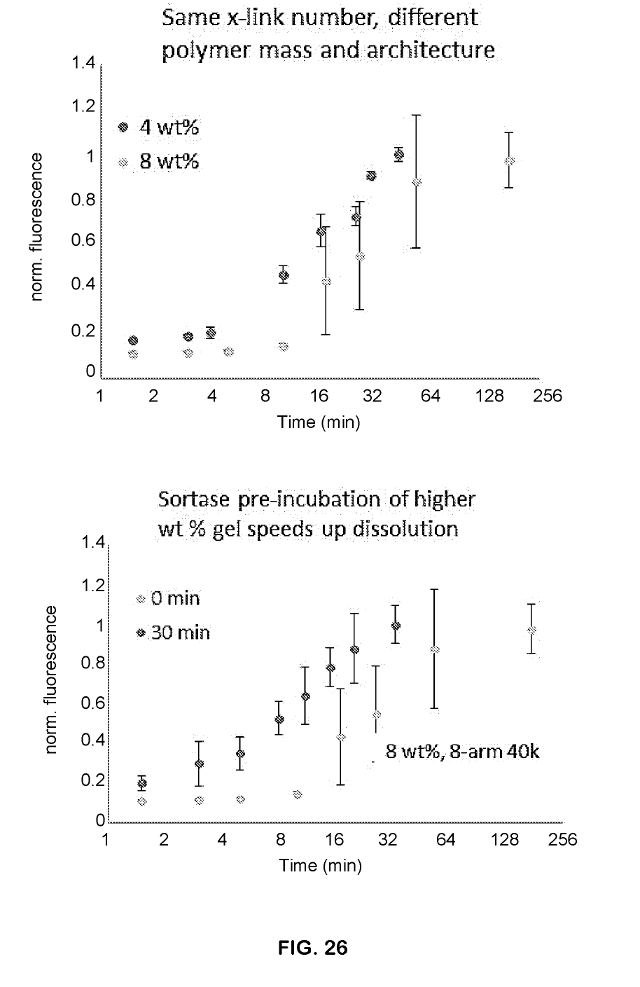

FIG. 26 shows that higher weight % gel dissolves more slowly, but pre-incubation with 50 uM sortase (as shown) before adding 18 mM GGG can speed up dissolution.

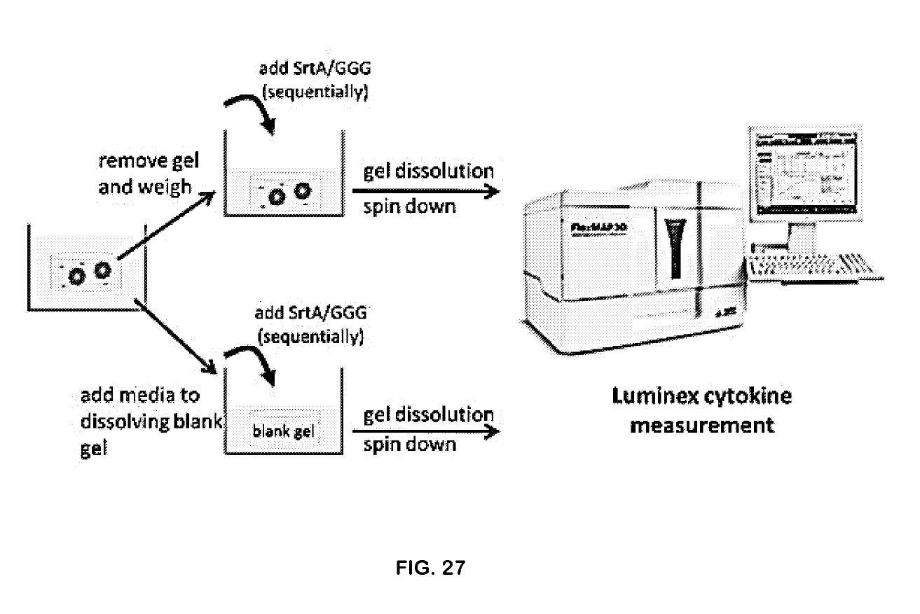

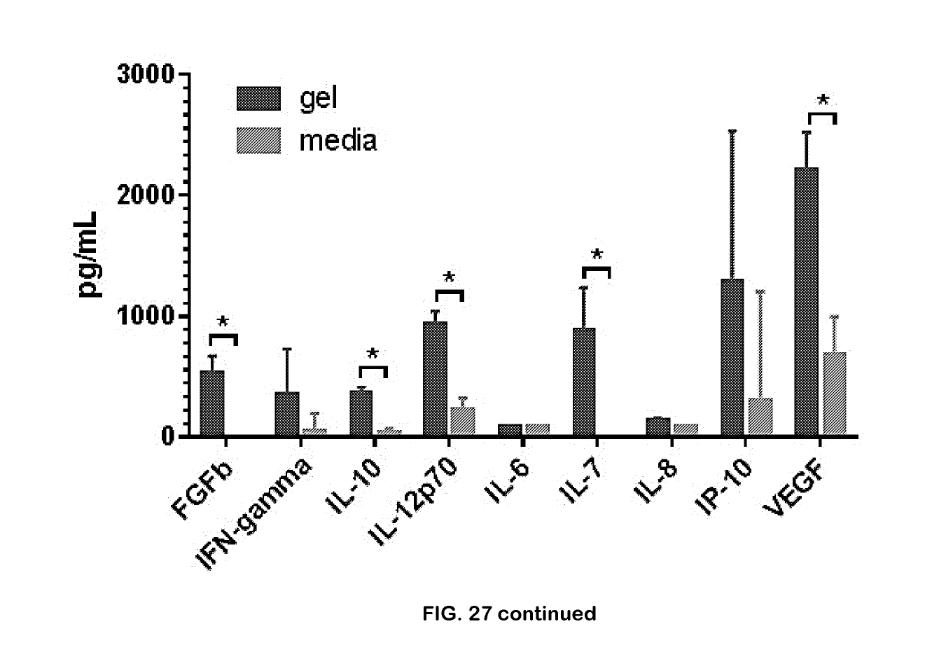

FIG. 27 at top panel shows a schematic for measuring cytokine concentrations inside the gel and in the culture media from an epithelial/stromal co-culture at 24 hrs. The bottom panel shows the results of the study.

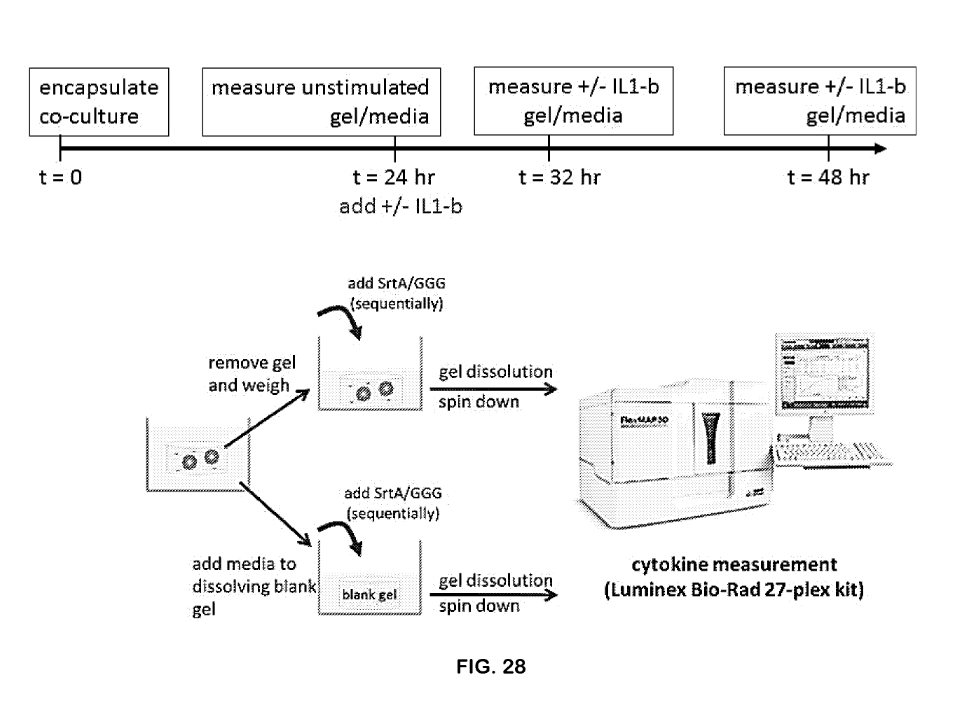

FIG. 28 illustrates the schematic and assay timeline for measuring a dynamic response of cytokine levels to IL1-b stimulation.

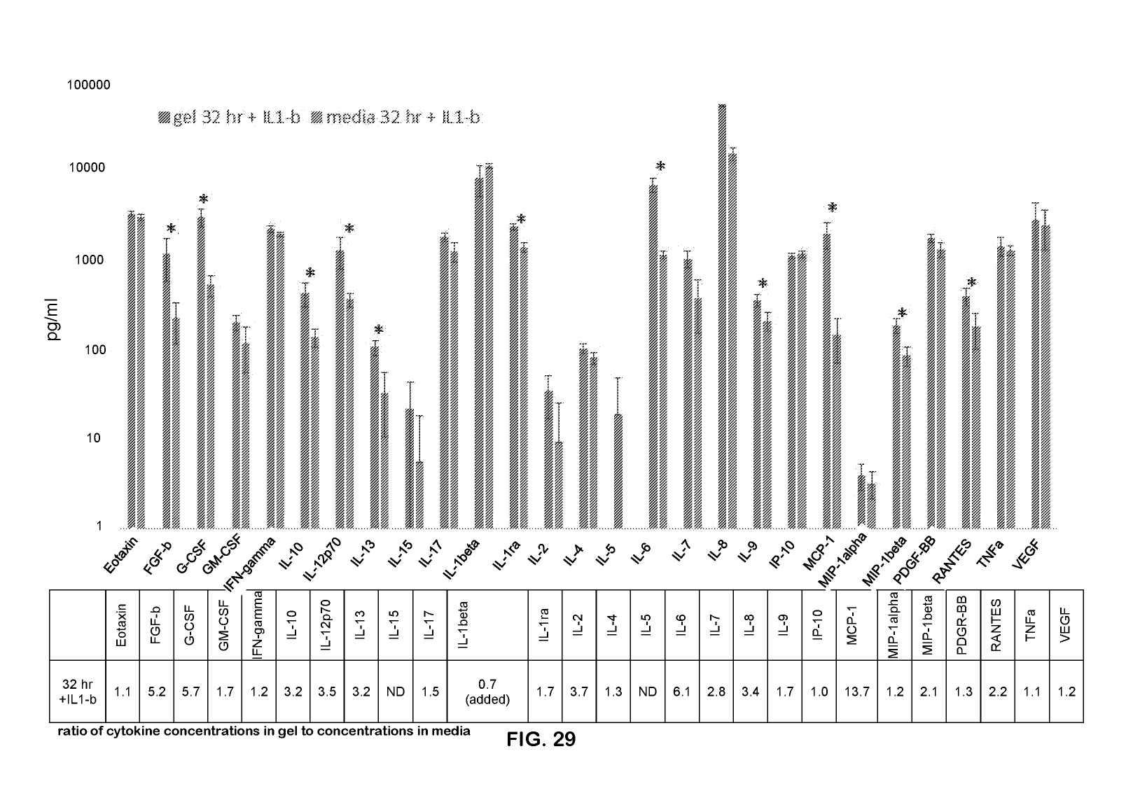

FIG. 29 shows various cytokine levels in the gel versus the media at the 32 hr time point (see FIG. 28 timeline) (representing 8 hr IL1-b stimulation). The ratios of various cytokine concentrations in-gel to media are shown. The study indicates that the differences in ratios cannot be explained solely by diffusion. Ratios at subsequent time points and varying conditions (e.g., at 32 hours +/−IL1-b; 48 hours +/−IL-1b) have also been measured (data not shown).

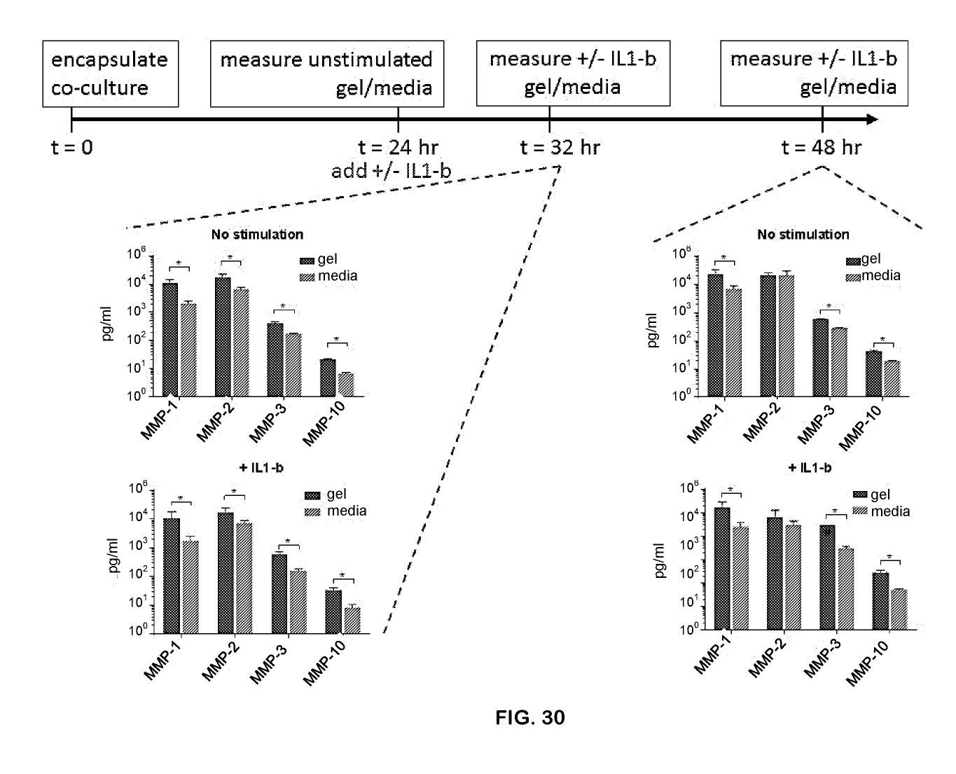

FIG. 30 shows MMP detection in the gel versus the media, as assayed and measured similarly as shown in FIGS. 28 and 29.

FIG. 31 shows TGFP detection in the gel versus the media, as assayed and measured similarly as shown in FIGS. 28 and 29.

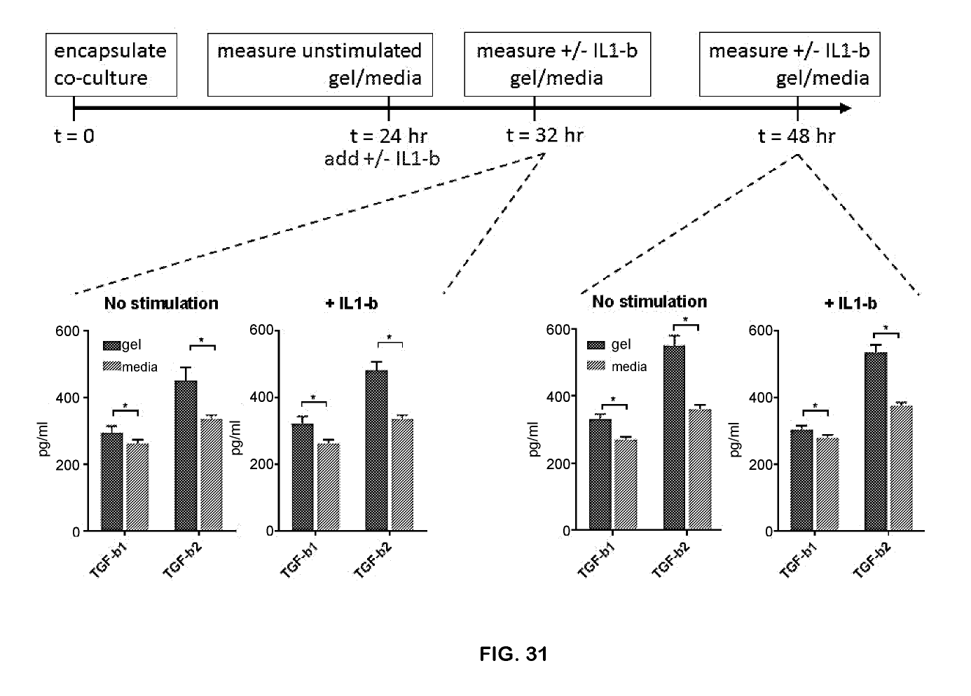

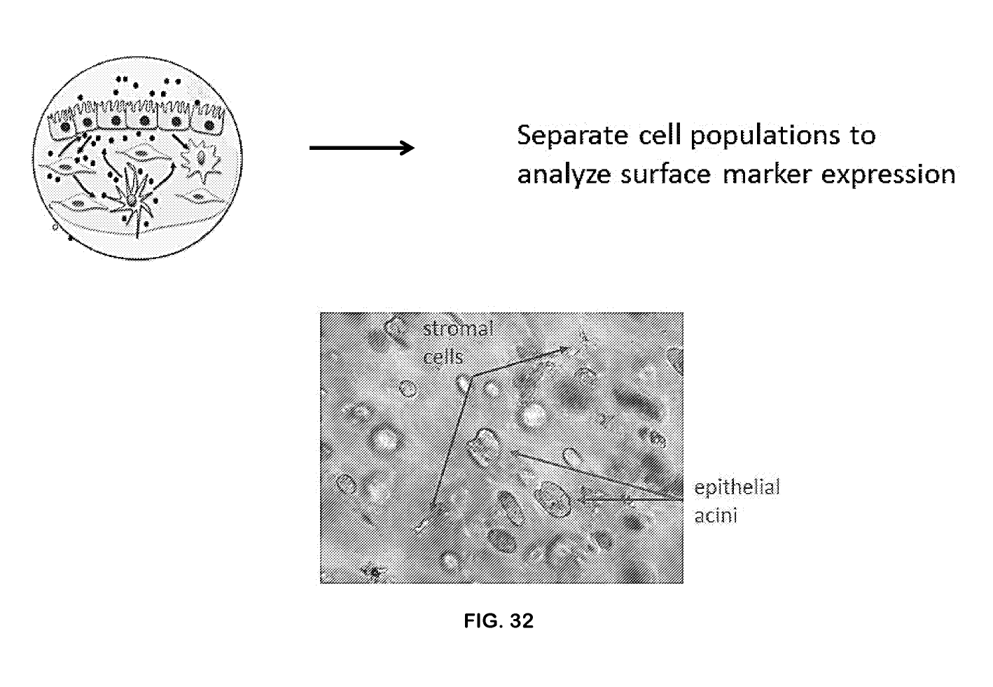

FIG. 32 illustrates formation of large acini (50-100 μm) by epithelial cells in 3D PEG gels, whereas stromal grow as single cells. SrtA dissolution preserves acinar structure morphology. Acini and single cells can be separated by size.

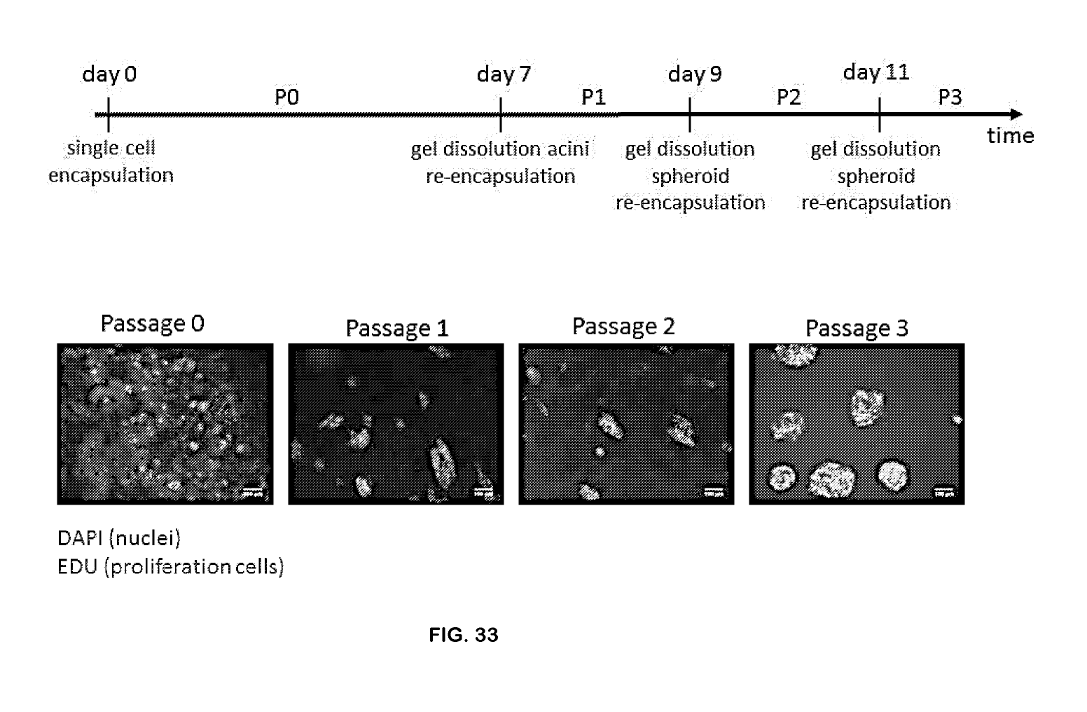

FIG. 33 demonstrates that 3D cultured epithelial acini maintain their morphology after SrtA gel dissolution and contain proliferating cells. The acinar structures were re-encapsulated in gels, and subsequently re-dissolved (“3D passaged”) three times (indicated as “Passage 1,” “Passage 2,” and “Passage 3”) without breaking the acini.

### DETAILED DESCRIPTION OF THE INVENTION

A description of example embodiments of the invention follows.

The present invention is based on crosslinking, modification, and/or dissolution of gels such as hydrogels (e.g., polyethylene glycol hydrogels) by transpeptidases (e.g., sortase A). Generally, transpeptidases catalyze a two-step reaction that begins with the activation of a transpeptidase recognition motif (recognized by the transpeptidase) through formation of an acyl-enzyme intermediate with concomitant release of one or more terminal amino acids. Subsequently, the activated motif can either be hydrolyzed with water or react with an acceptor substrate sequence (a second substrate sequence also recognized by the transpeptidase) having a nucleophilic amine terminus, thereby ligating the amine terminus of the acceptor substrate sequence to the recognition motif sequence. The peptide exchange process of transpeptidases is well characterized (Lupoli et al., JACS 133:10748-51, 2011, incorporated herein by reference in its entirety), and substrate sequences (i.e., the recognition motif and acceptor substrate sequence) are well known, or readily identifiable. Some examples of transpeptidases include, but are not limited to D-glutamyltransferase, peptidyltransferases, glutathione gamma-glutamyl cysteinyltransferase, gamma-glutamyltransferase, gamma-glutamylcyclotransferase, serine-type D-Ala-D-Ala carboxypeptidase, zinc D-Ala-D-Ala carboxypeptidase, glutathione hydrolase, and sortases including Sortase A, and Sortase B.

A notable aspect of SrtA-mediated reactions (and transpeptidases in general) is that the product formed can contain a sequence (e.g., LPRTGGG) that becomes itself a potential substrate (e.g., recognition motif). As described herein, using the reversibility of SrtA-mediated reactions (Liu et al., J. Org. Chem. 79, 487-492 (2014)), gels could be formed and dissolved in minutes while preserving cell viability, thus opening up the possibility that a single relatively low-cost, broadly accessible reagent can be used to create and break down highly tailored synthetic ECM. FIG. 19 provides an illustration of SrtA-mediated gel dissolution.

Thus, as described herein, the present invention provides methods of using transpeptidases (e.g., sortase A and sortase A variants) to effect crosslinking, modification, and/or dissolution of hydrogels. Sortases, in particular, are transpeptidases found in Gram-positive bacteria that anchor surface proteins to the bacterial cell wall. Sortase A (SrtA) catalyzes a peptide exchange process of the general form: (R)-LPXTG+GGG-(R)=(R)-LPXTGGG-(R)+G. As used herein, “X” in the context of an amino acid sequence can be any amino acid residue. Three known, engineered variants of Sortase A (SrtA), derived from Staphylococcus aureus, offer dramatically improved catalytic rate constants and tailored substrate specificity compared to wild type SrtA (the sequences of SrtA and the variants can be found in Chen et al., PNAS 108, 11399-11404, 2011, incorporated by reference herein in its entirety). SrtA and variants thereof are readily expressed in high yield as recombinant ˜25 kDa proteins (Chen, I. et al., PNAS 108: 11399-11404 (2011); Popp and Ploegh, Angew. Chem. Int. Ed. 50, 5024-5032 (2011); Chan, L. et al., PLoS ONE 2, e1164 (2007); all references incorporated by reference in their entireties). Moreover, sortase variants that recognize non-overlapping substrate sequences (e.g., recognition motifs and acceptor substrate sequences) have been described (e.g., Don et al., PNAS 111(37): 13343-13348, 2014; Raeeszadeh-Sarmazdeh, et al., Colloids and Surfaces B: Biointerfaces 128:457-463, 2015).

SrtA-mediated crosslinking provides many advantages over existing enzyme-mediated crosslinking strategies, owing at least in part to its: (i) specificity—the small peptide substrates of SrtA are rare in mammalian proteins, thus crosslinking of cells by the enzyme is avoided; (ii) increased catalytic rates—engineered mutants of SrtA with 100X greater catalytic efficiencies and tailored substrate affinities compared to wild type are available; (iii) increased diffusion rates—SrtA is relatively small (25 kDa) relative to other crosslinking enzymes; and (iv) availability—SrtA mutants can easily be produced recombinantly in high yield.

Accordingly, in one embodiment, the present invention provides a hydrogel comprising one or more scaffold macromers crosslinked to a mixture of peptides, wherein all or a portion of the peptides in the mixture comprise a recognition motif cleavable by a transpeptidase, as described herein.

In some embodiments, the hydrogel of the present invention includes, for example, gels formed as a result of e.g., norbornene-thiolene or Michael-type (vinyl sulfone) crosslinking chemistry, as well as gels formed by sortase. Thus, as used herein, the term “crosslinked” in the context of “macromers crosslinked to a mixture of peptides” includes the joining of a macromer to a peptide as a result of chemical crosslinking (e.g., norbornene-thiolene or Michael-type (vinyl sulfone) crosslinking chemistry), or transpeptidase reaction.

In certain embodiments, a portion of the peptides in the mixture is crosslinked to one or more macromers at its N-terminus, and is free at its C-terminus. In a related embodiment, the hydrogel comprises a pendant transpeptidase substrate sequence, which refers to a sequence that has one end that is not joined by a macromer and is thus accessible by a transpeptidase. The advantages of such a system is to, e.g., readily tether and cleave a biomolecule of interest as desired.

In other embodiments, the present invention also provides a method of forming a hydrogel dissolvable by a transpeptidase, said method comprising: combining 1) a mixture of peptides, wherein all or a portion of the peptides in the mixture comprise a recognition motif cleavable by a transpeptidase, each peptide having a first crosslinking moiety; 2) one or more scaffold macromers having a second crosslinking moiety; and 3) a suitable crosslinking agent under suitable conditions that promote crosslinking of the first and second crosslinking moieties, thereby forming a hydrogel dissolvable by a transpeptidase. In certain embodiments, the transpeptidase is a sortase or a sortase variant. In one embodiment, the sortase is Sortase A (SrtA).

As used herein, a “mixture of peptides” refers to a collection of peptides wherein the mixture can be a collection of a homogeneous population of peptides, or a mixture of two or more different peptides having different amino acid sequences.

As used herein “variant” refers to mutants and modified versions of a protein, and also includes fragments that retain the same or similar activity of the full-length protein.

As used herein, “SorA”, “SrtA” and “Sortase A” are used interchangeably. Unless indicated otherwise, “sortase” refers to the general class of sortase enzymes, which includes Sortase B, for example, as well as Sortase A and variants.

As used herein, “having” is used interchangeably with “comprising”.

In certain embodiments, the peptide can be a linear peptide that comprises a crosslinking moiety on each end. In this scenario, the peptide is flanked by a macromer on each end. In other embodiments, the peptide can be a branched peptide that comprises at least 3 crosslinking moieties (i.e., reactive groups) capable of forming crosslinks with one or more macromers having crosslinking moieties. In this scenario, all or a portion of the branches can have a crosslinking moiety and/or all or a portion of the branches can have one or more sequences that add a functional feature, e.g., substrate sequence for a protease. By way of example, if the peptide has three branches, two of the branches can comprise a transpeptidase recognition motif followed by a crosslinking moiety (and thus join with macromers), while one of the branches has a terminal transpeptidase recognition motif that does not have a crosslinking moiety (and thus serve as a pendant transpeptidase substrate sequence as described herein).

A “crosslinking moiety” as used herein refers to any known suitable reactive groups used in polymer chemistry, as exemplified herein. Suitable conditions for effecting crosslinking using various crosslinking groups are known in the art. “Crosslinking moiety” is often used interchangeably with “crosslinking groups” and “reactive groups.”

In certain embodiments, the recognition motif comprises a sequence selected from the group consisting of: LPXSG, LPXTG, and LAXTG. Other recognition motifs recognized by various sortase variants can also be used.

As described herein, transpeptidases catalyze a two-step reaction that begins with the activation of a transpeptidase “recognition motif” (recognized by the transpeptidase) through formation of an acyl-enzyme intermediate with concomitant release of one or more terminal amino acids. Subsequently, the activated recognition motif can either be hydrolyzed with water or react with an “acceptor substrate sequence” (a second substrate sequence also recognized by the transpeptidase) having a nucleophilic amine terminus, thereby ligating the amino terminus of the acceptor substrate peptide sequence to the recognition motif sequence. In the context of a transpeptidation reaction, the “recognition motif” and “acceptor substrate sequence” can be used interchangeably. To illustrate, if a transpeptidase recognizes a recognition motif of ABCDE, and an acceptor substrate sequence of FGHIJ, the “recognition motif” comprises at least ABCDE (e.g., (R)-ABCDE, where R is any moiety, including a polymer or protein), and the “acceptor substrate sequence” comprises at least FGHIJ (e.g., FGHIJ-(R), wherein R is any moiety, including a polymer or protein). The “recognition motif” and “acceptor substrate sequence” as used herein may be interchangeable, so long as the necessary terminal ends on the recognition motif or the acceptor second substrate sequences are present for a transpeptidase to catalyze the reaction. However, one of the substrate sequences requires a terminal nucleophilic amine (generally referred to as the “acceptor” substrate) to complete the two-step transpeptidase reaction. Generally, as used herein, “substrate sequence” refers to either the recognition motif or the acceptor substrate sequence.

As described herein, “dissolvable” refers to partial or complete dissolution of the gel, and is not limited to a condition in which the gel is completely liquefied. In certain embodiments, partial dissolution of the gel can render the gel having a changed physical property (e.g., decreased gel stiffness and/or density), while maintaining a gel structure. Methods of assessing gel stiffness and/or density are known in the art. For example, rheological measurements (e.g., of loss and storage moduli) and atomic force microscopy can be performed. Swelling of the gel can be measured as an indicator of a change in crosslinking density.

In some embodiments, the portion of the peptides in the mixture (e.g., a first population of peptides) that comprises a recognition motif cleavable by a first transpeptidase can be about 0.001% to about 80% by weight % of the total mass of polymers used to form the hydrogel. In particular embodiments, the portion of the peptides in the mixture that comprises a recognition motif cleavable by a first transpeptidase can be about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 11%, about 12%, about 13%, about 14%, about 15%, about 16%, about 17%, about 18%, about 19%, about 20%, about 22%, about 24%, about 26%, about 28%, about 30%, about 35%, about 40%, about 45%, about 50%, about 55%, about 60%, about 65%, about 70%, about 75%, or about 80%. In a particular embodiment, about 0.001% to about 80% of the peptides in the mixture comprise a recognition motif cleavable by a first transpeptidase. In a related embodiment, the remaining portion of the peptides in the mixture (e.g., a second population of peptides) is not cleavable by the first transpeptidase. In this example, the second population of peptides comprises a recognition motif that is cleavable by another transpeptidase (e.g., a SrtA variant that does not recognize the recognition motif in the first population of peptides). In other related embodiments, as would be appreciated by those of skill in the art, the mixture of peptides can further comprise additional populations of peptides, each having a unique transpeptidase recognition motif that is cleavable by a unique sortase, including branched peptides.

By way of example, the present method enables the formation of a hydrogel wherein, e.g., 10% of the peptides that occur at the crosslinks of macromers contain a recognition motif for a hypothetical transpeptidase A, while the remaining 90% of the peptides that occur at the crosslinks of macromers do not contain a recognition motif for any transpeptidase (and instead comprises a non-functional sequence, or other functional sequences as described herein—e.g., a sequence cleavable by a protease and/or a cell adhesion domain). By doing so, treating the hydrogel with transpeptidase A (and its acceptor substrate sequence) will cleave 10% of the peptides in the hydrogel (and therefore break the crosslinks between macromers joined by this peptide). Depending on the crosslink density in a manner determined by Flory theory, or by experimental determination, the proportion of peptides that contain the transpeptidase A recognition motif can be fine-tuned to the desired physical property of the gel. For example, breaking 10% of the hydrogel's crosslinks can result in a softer gel. Thus, the maximum percentage of crosslinks that can be broken while retaining an intact gel depends on the cross link density.

As another example, the present method enables the formation of a hydrogel wherein, e.g., 10% of the peptides that occur at the crosslinks of macromers contain a recognition motif for hypothetical transpeptidase A, while the remaining 90% of the peptides that occur at the crosslinks of macromers contain a recognition motif for hypothetical transpeptidase B (which does not cleave the recognition motif for transpeptidase A). By doing so, treating the hydrogel with transpeptidase A (and its acceptor substrate sequence) will cleave 10% of the peptides in the hydrogel (and therefore break the crosslinks between macromers joined by this peptide). If it is subsequently desired to completely dissolve the gel (e.g., to release encapsulated cells for anlysis), then the hydrogel can be treated with transpeptidase B (and its acceptor substrate sequence) to cleave the remaining peptides that have not been cleaved by transpeptidase A. Again, the proportions of peptides that can be cleaved by various transpeptidases can be optimized depending on the desired effect.

In other embodiments, the mixture of peptides can further comprise a terminal peptide having a recognition motif cleavable by a transpeptidase, said terminal peptide having a crosslinking moiety on one end. As used herein, a “terminal peptide” refers to a population of peptides having a crosslinking moiety on one end of the peptide (e.g., C-terminus of the peptide). Thus, in this embodiment, addition of a terminal peptide in the present method allows the formation of a hydrogel having a reactive (free) transpeptidase recognition motif, allowing functional groups (e.g., biomolecule such as polypeptides or signaling molecules) to be added and removed using a transpeptidase (preferably a transpeptidase that only recognizes and cleaves the terminal peptide) within an intact gel. In certain embodiments, the terminal peptide can comprise a biomolecule preattached prior to forming the hydrogel. That is, the biomolecule attachment (functionalization) can take place after forming the hydrogel, or a terminal peptide having a biomolecule preattached can be used to make the hydrogel.

In some embodiments, one or more peptides in the mixture of peptides further comprise a sequence cleavable by a protease, including, for example, endoproteases (e.g., serine proteases, cysteine proteases, aspartic acid proteases), and metalloproteases (e.g., matrix metalloproteases and A Disintegrin And Metalloproteinase (ADAM)). That is, in addition to a transpeptidase recognition motif, one or more peptides in the mixture of peptides can further comprise a sequence cleavable by a protease. In certain embodiments, the mixture of peptides can comprise one or more populations of peptides that contain a sequence cleavable by a protease (but without a transpeptidase recognition motif) and one or more populations of peptides that comprise at least a transpeptidase recognition motif. Those of skill in the art can readily identify and design sequences that can be cleaved by such proteases.

In certain embodiments, the hydrogels of the present invention provide a three-dimensional environment in which, e.g., cellular events can be studied. For example, the hydrogels of the present invention allow the three-dimensional “culture” of cells in a matrix environment that can be designed to mimic the extracelluar environment, by providing e.g., cell adhesion domains and cell-driven dynamic matrix remodeling. Thus, in some embodiments, one or more populations of peptides in the mixture of peptides can also comprise cell adhesion domains. Cell adhesion domains include, for example, derivatives of the adhesive sequence RGD, derivatives of the adhesion sequence RGD that contain the synergy site PHSRN, derivatives of the adhesive sequence FOGER derived from collagen I, and polylysine.

In one embodiment, one or more populations of peptides in the mixture of peptides comprise the amino acid sequence GCRDLPRTGGPQGIWGQDRCG (SEQ ID NO: 1).

In other embodiments, the method further comprises combining a cell, a tissue, or an organ, or any combination thereof, thereby encapsulating such constituents into the hydrogel.

As appreciated by those of skill in the art, various scaffold macromers can be used in the present methods, provided that the macromer comprises, on average, at least three reactive groups (e.g., crosslinking moieties). In certain embodiments, the macromer comprises, on average, 3-1000 reactive groups. In some embodiments, it is possible to form a hydrogel using a scaffold macromer that has two reactive groups (e.g., a linear bifunctional PEG) if the mixture of peptides comprises one or more populations of branched peptides that have, on average, 2-3 or more reactive groups. As would be appreciated by those of skill in the art, hydrogel formation can be achieved when there is an average of 2-3 reactive groups on the macromer. Thus, by way of example, a bifunctional PEG having a crosslinking moiety on each end (i.e., two total, with one on each end) can be mixed with a peptide (e.g., branched peptide) having, on average, 2-3 reactive crosslinking moieties in the presence of a crosslinking agent under suitable conditions to form a hydrogel. As those of skill in the art would understand, conditions suitable for gel formation using various macromer and peptide components can be experimentally determined.

A scaffold macromer (also referred to herein as “polymer”) can be selected from any one or more of polyethylene glycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin. Other suitable scaffold macromers are known in the art (Kadajji and Betageri, Polymers 3:1972-2009, 2011). In some embodiments, PEG can be linear or branched. In a particular embodiment, the PEG is an 8-arm PEG having vinylsulfone or norbornene reactive groups. In other embodiments, the macromer can be any water-soluble polypeptide, in particular, a branched polypeptide.

In some embodiments, more than one macromer (i.e., more than one type of macromer) can be used for the present methods. In certain embodiments, the macromer is an 8-arm PEG having 8 crosslinking moieties.

In other embodiments, the present invention also provides a method of dissolving a hydrogel comprising scaffold macromers crosslinked to a mixture of peptides, wherein all or a portion of the peptides in the mixture comprise a recognition motif cleavable by a transpeptidase, said method comprising treating the hydrogel with a first transpeptidase and a peptide comprising an acceptor substrate sequence of the first transpeptidase under conditions that promote dissolution of the hydrogel, thereby dissolving the hydrogel. In certain embodiments, the acceptor substrate sequence comprises NH₂-(G)ₙ, wherein n is equal to or greater than 1.

In various embodiments, as described herein, the hydrogel is pretreated with a transpeptidase (e.g., sortase) prior to treating with the peptide comprising an acceptor substrate sequence.

In various other embodiments, transpeptidases, and particularly SrtA, can be used to reversibly functionalize gels with biomolecules (e.g., protein ligands such as growth factors), which can be readily released in localized fashion. That is, by providing the necessary transpeptidase recognition motif (e.g., LPXTG or GGG) at the terminus of a protein or peptide sequence, the transpeptidase can efficiently tether the protein to a polymer in a hydrogel, so long as the polymer also includes a pendant (free and reactive) transpeptidase substrate sequence that can react with a terminus of the protein to be tethered. Accordingly, as described herein, the present methods allow for an inexpensive, versatile, and facile process for incorporating (and selectively releasing) biomolecules within hydrogels, providing tailored, modified hydrogels that possess intrinsic biological function. The resulting modified hydrogels are potentially capable of eliciting a biological response akin to a native extracellular matrix, providing a 3D in vitro environment.

As described herein, the present method relates to a method of forming gels, e.g., hydrogels that comprise a transpeptidase substrate sequence (transpeptidase recognition motif or acceptor substrate sequence) contained at a crosslink bridge. In some embodiments, the substrate sequence occurs at the junction where two polymers crosslink (or join). In certain embodiments, the gel can be fully dissolved by a transpeptidase under suitable conditions.

Hydrogels that comprise a transpeptidase substrate sequence that is within the crosslink bridge (crosslink junction) can be formed using any polymer crosslinking methods known in the art, and as described in references cited herein. Merely to illustrate, in one embodiment, a gel crosslinked in the following configuration: polymer-LPRTGGG-polymer (where “polymer” refers to “macromer” as used herein) can be formed using conventional crosslinking methods by combining polymer-A+B-LPRTGGG-B+A-polymer, where A and B represent any known crosslinking moiety. Such gels may be readily degraded and dissolved in the presence of a transpeptidase and a peptide that comprises a transpeptidase acceptor substrate sequence (e.g., sortase and NH₂-GGG). As described herein, the peptide represented by LPRTGGG in this example can further comprise additional functional sequences. Moreover, such a reaction can be performed with a mixture of peptides having desirable sequences, as described herein.

Alternatively, it may be desirable to achieve partial degradation of the hydrogel. This could be achieved by controlling the initial crosslinking reaction wherein a mixture of B-LPRTGGG-B and B-B is crosslinked with polymer-A and A-polymer, where again, A and B represent any known crosslinking moiety. Thus, by controlling the ratio of B-LPRTGGG-B and B-B in the reaction, a gel having desirable dissolution properties can be achieved. In certain embodiments, such gels encapsulate biomaterials, such as cells, tissues, or organs. In other embodiments, it is also possible to form a gel crosslinked in the configuration polymer-LPRTGGG-polymer by using transpeptidase (e.g., sortase) mediated ligation. For example, polymer-LPRTGGG+GGG-polymer may be combined in the presence of sortase to form hydrogels crosslinked in the configuration polymer-LPRTGGG-polymer. Further, the transpeptidase substrate sequence need not be directly conjugated to the polymer, but might also be in series with other functionality such as, e.g., known proteolytically degradable peptides often integrated into crosslinked gels.

In another embodiment, the present invention provides methods of forming a hydrogel that comprises a pendant transpeptidase recognition motif, said method comprising: combining a one or more scaffold macromers having a first crosslinking moiety, a peptide comprising a transpeptidase recognition motif having a second crosslinking moiety at its N-terminus, and a suitable crosslinking agent under conditions that promote crosslinking of the first and second crosslinking moieties, thereby forming a hydrogel that comprises a pendant transpeptidase recognition motif. Suitable crosslinking agents are described herein and well known in the art. Further, methods of crosslinking and suitable conditions that promote crosslinking are well known.

As used herein, “pendant transpeptidase substrate sequence” refers to a sequence that has one end that is not joined by a macromer and is thus accessible by a transpeptidase. The advantages of such a system is to, e.g., readily tether and cleave a biomolecule of interest as desired.

As exemplified herein, an 8-arm PEG-acrylate can be combined with a peptide having the sequence GCRE-LPRTGGGK-NH2, together with 4-arm PEG-thiol to form a hydrogel having a pendant transpeptidase substrate sequence. The order of adding each component (e.g., macromer and peptide) is not important, so long as the components are combined in a manner that forms a gel. Conditions for crosslink formation that yields a hydrogel are known, and can be determined experimentally.

In certain embodiments, the one or more macromers having a crosslinking moiety can be present as part of a pre-formed gel, to which a peptide comprising a transpeptidase recognition motif having a second crosslinking moiety at its N-terminus can be added, resulting in a pendant hydrogel.

Hydrogels having a pendant transpeptidase substrate sequence allow for targeted addition, removal, or exchange of biomolecules selectively to the hydrogel. To illustrate, a gel of the configuration: (polymer gel)-LPRTG[X1], where X1 is nothing or a biomolecule, can be readily made using traditional polymer crosslinking methods. For example, polymer-A+B-LPRTGGG[X1], wherein A and B represent any known crosslinking moieties, can be crosslinked using any of the known crosslinking methods to form (polymer gel)-LPRTG[X1]. A gel of this configuration is defined as having a pendant (terminal) transpeptidase substrate sequence (here, as exemplified, a sortase substrate sequence). Once formed, reacting (polymer gel)-LPRTG[X1]+GGG-[X2] and sortase will produce (polymer gel)-LPRTGGG-[X2]+G[X1]. Using this general approach, a functional ligand may be readily tethered to the hydrogel when [X1] is nothing, and [X2] is a functional biomolecule (e.g., a growth factor or an adhesion molecule). Alternatively, a functional biomolecule may be readily removed from the hydrogel when [X1] is a functional biomolecule and [X2] is nothing. Additionally, a biomolecule tethered and incorporated into the gel may be exchanged for another biomolecule when [X1] is functional biomolecule A and [X2] is a functional biomolecule B. Accordingly, hydrogels comprising a pendant transpeptidase substrate sequence can be readily formed, and used to easily functionalize the hydrogel with a biomolecule, remove the functional biomolecule, or exchange the functional biomolecule for another.

In other embodiments, hydrogels of the present invention may be modified and functionalized to contain not just tethered biomolecules, but also proteolytic substrate sequences in addition to transpeptidase substrate sequences. For example, as illustrated in FIG. 18, a matrix metalloprotease (MMP) substrate sequence is incorporated into the hydrogel in addition to the sortase substrate sequence. Incorporation of the MMP substrate sequences encourages proper cell growth in a 3D hydrogel space by mimicking the ECM. Over time, cells produce proteases (e.g., MMPs) that locally degrade the MMP substrate crosslink, which allow the cells to spread or even migrate through the gels (see, e.g., Fonseca et al., Prog. in Poly. Sci. 39(12):2010-29, 2014).

In another embodiment, the present invention provides methods for forming hydrogels through a transpeptidase-mediated reaction. Thus, it is also possible to form a gel crosslinked in the configuration polymer-LPRTGGG-polymer by using sortase-mediated ligation. Accordingly, in a further embodiment, the present invention relates to methods of forming a functionalized hydrogel, said method comprising: combining a first scaffold macromer having a terminal first transpeptidase recognition motif; a second scaffold macromer having a terminal transpeptidase acceptor substrate sequence; one or more biomolecules having a terminal transpeptidase recognition motif or acceptor substrate sequence; and a transpeptidase under conditions that promote transpeptidase ligation of the transpeptidase recognition motif with the acceptor substrate sequence, thereby forming a functionalized hydrogel.

To illustrate, for example, polymer-LPRTG+GGG-polymer may be combined in the presence of sortase to form hydrogels crosslinked in the configuration polymer-LPRTGGG-polymer. Furthermore, the hydrogel may be easily modified and functionalized in a one-step approach by combining, for example, polymer-LPRTG+GGG-polymer+(biomoleculeA-LPRTG and/or GGG-biomoleculeB), wherein the biomolecule may not only be a protein or peptide, but a proteolytically degradable peptide sequence, depending on the functionality desired. Accordingly, any suitable combination of various arrangements of polymers and biomolecules having the appropriate transpeptidase substrate sequences may be used to control the nature of functionality desired.

In certain embodiments, the transpeptidase substrate sequence is attached to the polymer at the N-terminus of the substrate sequence, allowing the sequence to be reactive and accessible to sortase for transpeptidation.

In forming gels according to the present methods, the concentration of the transpeptidase substrate sequence (e.g., LPRTG) can be varied, as desired. For example, a transpeptidase substrate sequence (e.g., LPRTG) may be incorporated into a gel at a concentration of 25 μM, 50 μM, 75 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, or 500 μM. Each system may be assayed for the optimal substrate sequence concentration in the gel for any given application.

In any of the foregoing embodiments, a variety of other transpeptidase substrate sequences (e.g., LPXSG, LPXTG, and LAXTG) can be readily applied to the present methods. Additionally, any suitable substrate sequence comprising a nucleophilic amine may also be used (e.g., a chemical compound) in place of, e.g., GGG, as described in, e.g., Baer et al., Organic & Biomolecular Chemistry, 12:2675, 2014.

In any of the foregoing embodiments, any suitable biomaterial, e.g., cells, tissue, organs, may be encapsulated in the hydrogels of the present invention. In further embodiments, beads comprising a transpeptidase substrate sequence at the surface of the beads may also be encapsulated in the hydrogels, to effect selective and local degradation of the hydrogel upon treatment with, e.g., sortase and NH₂-GGG.

In other embodiments, the present invention provides a method of dissolving a hydrogel, said method comprising: treating a hydrogel comprising a transpeptidase recognition motif with a transpeptidase and a peptide comprising an acceptor substrate sequence under conditions that promote dissolution of the hydrogel, thereby dissolving the hydrogel. In some embodiments, the dissolution is complete. In other embodiments, the dissolution is partial.

In certain embodiments, the present invention provides a method of dissolving a hydrogel, said method comprising treating a hydrogel with a transpeptidase, in the absence of an acceptor substrate sequence under conditions that promote dissolution of the hydrogel, wherein the hydrogel comprises a transpeptidase recognition motif. In this example, the transpeptidase activates the recognition motif in the hydrogel to form an acyl-enzyme intermediate, which is subsequently hydrolyzed.

In certain embodiments, the hydrogel comprises a transpeptidase substrate sequence (recognition motif or acceptor substrate sequence) that is within the crosslink bridge. That is, the substrate sequence occurs at the junction where two polymers crosslink (or join).

In certain embodiments, the present invention provides a method of dissolving a hydrogel, said method comprising: treating a hydrogel comprising a sortase recognition motif with a sortase and a peptide comprising an acceptor substrate sequence under conditions that promote dissolution of the hydrogel, thereby dissolving the hydrogel.

In certain embodiments, the hydrogel comprises a transpeptidase substrate sequence that is within the crosslink bridge. That is, the substrate sequence occurs at the junction where two polymers crosslink (or join).

In certain embodiments, the sortase is Sortase A. In other embodiments, the sortase is a modified Sortase A as described in Chen, I. et al., PNAS 108: 11399-11404 (2011). However, other sortases can readily be applied to the present methods, so long as the substrate sequences are identifiable. Methods for producing sortase variants and determining specificities of variants have been described (Dorr et al., PNAS 111(37):13343-48, 2014, incorporated by reference herein in its entirety).

In another embodiment, the sortase recognition motif comprises a sequence selected from the group consisting of: LPXSG, LPXTG, and LAXTG. However, other sortase substrate sequences are also possible (see, e.g., Dorr et al, PNAS 111(37):13343-48, 2014, incorporated by reference herein in its entirety). In one embodiment, the first sortase substrate sequence within the hydrogel is LPXTG, where X is R or E.

Additionally, as described herein, the peptide used to treat the hydrogel comprises an acceptor substrate sequence. In one embodiment, the peptide comprises NH₂-(G)ₙ (where n is equal to or greater than 1). In one embodiment, the peptide comprises NH₂-GGG. However, it is possible to use other sortase substrate sequences as part of the peptide (see, e.g., Baer et al., Organic & Biomolecular Chemistry, 12:2675, 2014) to effect hydrogel dissolution. Further, the peptide may comprise other groups in addition to the sortase acceptor substrate sequence, so long as the peptide has a free nucleophilic NH₂-(G)ₙ terminus. For example, the peptide may be of the structure NH₂-(G)ₙ-(R), wherein R is a moiety that increases or decreases the rate of gel dissolution. Thus, the peptide may be designed to fine-tune the rate of dissolution.

In particular embodiments, the hydrogel is treated with sortase at a concentration of approximately 2 μM, 5 μM, 8 μM, 10 μM, 15 μM, 20 μM, 25 μM, 5004, 7504, 100 μM, 125 μM, 150 μM, 175 μM, 200 μM, 225 μM, 250 μM, 275 μM, 300 μM, 325 μM, 335 μM, 350 μM, 375 μM, 400 μM, 425 μM, 450 μM, 475 μM, or 500 μM. In particular embodiments, the hydrogel is treated with the peptide comprising an acceptor substrate sequence at a concentration of 100 μM, 150 μM, 200 μM, 250 μM, 300 μM, 400 μM, 500 μM, 600 μM, 700 μM, 800 μM, 900 μM, 1 mM, 3 mM, 5 mM, 10 mM, 15 mM, 17 mM or 20 mM. Generally, the kinetics of dissolution can be fined-tuned by providing more or less of the sortase and peptide. Additionally, preincubating the gel with sortase will allow rapid dissolution with a lower concentration of sortase (e.g., approximately 10 μM).

In certain embodiments, the hydrogel to be dissolved encapsulates a biomaterial such as a cell, a tissue, or an organ.

In some embodiments, dissolution of the gel occurs rapidly. For example, a polyethylene glycol (PEG) hydrogel having a 20 μl volume completely dissolves in less than 5 minutes (FIG. 19). However, complete dissolution is not required; rather, dissolution that allows the release of a biomaterial (e.g., cells, tissue, organ) is sufficient.

In some embodiments, it may desirable to dissolve the hydrogel using sortase in conditions that promote hydrogel dissolution, but in the absence of a peptide that comprises a sortase acceptor substrate sequence (e.g., a GGG peptide). In such an embodiment, hydrogel dissolution is expected to occur more slowly as compared to hydrogel dissolution in the presence of a peptide that comprises a sortase substrate sequence.

In other embodiments, the present invention provides a kit for hydrogel formation comprising: an isolated transpeptidase enzyme; and a plurality of scaffold macromers, wherein said plurality comprises at least a first macromer having a terminal transpeptidase recognition motif (e.g., crosslinked or bound to the macromer at the N-terminus of the recognition motif, while the C-terminus is free and accessible to a transpeptidase), and at least a second macromer having a terminal transpeptidase acceptor substrate sequence.

In some embodiments, the first and second macromer are different from each other (e.g., one is PEG, while the other is dextran). In other embodiments, the first and second macromer are the same.

In further embodiments, the kit further comprises a suitable buffer, as described herein. Suitable buffer conditions used for crosslinking are well known in the art.

In one embodiment, the transpeptidase is a sortase, or more specifically, a modified Sortase A. In another embodiment, the second transpeptidase substrate comprises NH₂-(G)ₙ, where n is equal to or greater than 1. In some embodiments, the second transpeptidase substrate comprises NH₂-triglycine (GGG).

In another embodiment, the present invention relates to gels formed by any of the methods described herein.

The gels of the present invention, as applied to any of the foregoing embodiments, may be formed using any of the polymers, or “scaffold macromers”, described herein or known in the art. Examples of such scaffold macromers include, but are not limited to, polyethylene glycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin, or combinations thereof In another embodiment, copolymers that comprise at least one hydrophilic polymer may also be used, alternatively or in combination with scaffold macromers.

### Exemplification

The following Examples are merely illustrative, and are not intended to limit the scope or content of the invention in any way.

### Example 1

### Hydrogel Formation Using SrtA

End-functionalized PEG macromers can be rapidly crosslinked by SrtA to yield mechanically robust hydrogels that preserve viability of encapsulated cells. FIG. 17 shows a schematic of sortase-mediated bulk crosslinking to form PEG hydrogels. As shown in FIG. 17, PEG polymers having C-LPRTG-fam at their ends crosslink with PEG polymers having GGG-C at their ends in the presence of sortase, forming a gel. Cells or tissue may be added to the polymer mixture with sortase to encapsulate them. The encapsulated cells or tissue may then be assayed for viability and/or for functional properties according to the methods described herein.

Three different SrtA mutants have comparable values of k_{cat} (4.8-5.4 s⁻¹), but different individual substrate affinities (Table 1 below from Chen et al., PNAS 108, 11399-11404, 2011), and therefore different relative forward and reverse kinetics. Other modified sortases with different kinetics and substrate specificity may also be used in the present methods.

TABLE 1
Comparison of k_{cat} and substrate affinities for SrtA mutants

| | K_{m, LPTG} | K_{m, GGG} | K_{cat/Km, LPTG} |
|---|---|---|---|
| Mutant | (mM) | (mM) | (M⁻¹s⁻¹) |
| Triple (SrtA-3M) | 0.6 | 1.8 | 8600 |
| Tetra (SrtA-4M) | 0.2 | 4.8 | 28000 |
| Penta (SrtA-5M) | 0.2 | 2.9 | 23000 |

The SrtA triple mutant (“SrtA-3M”) was used for gelation studies (Chen et al., PNAS 108, 11399-11404, 2011; Krueger et al., Angew. Chem. Int. Ed. 53, 2662-2666, 2014). A 40,000 Mw, 8-arm PEG (Jenken), modified with vinyl sulfone (VS), was functionalized with either -CLPRTG or GGGC- in HEPES buffer at pH 8 for 4 hours at a 1:1.2 VS:peptide ratio. Product was dialyzed for 3 days (4° C.), frozen (−80° C.), and lyophilized. The functionalized macromers were redissolved in a HEPES buffer (pH 7.4) with CaCl₂ (10 mM) and mixed with SrtA-3M to yield a 5% (w/w) macromer solution containing 650 μM each of the -LPRTG and -GGG functionalized PEG ends, and either 135 or 338 μM SrtA-3M. Oscillatory shear rheological measurements (0.5 Hz with a strain of 1%) confirmed gel formation of the precursor solutions (FIG. 1) as indicated by the rapid rise in storage modulus G′, which reached a plateau in under 4 min for the 338 μM SrtA-3M concentration. The final modulus was comparable for both SrtA-3M concentrations, but the gel formation kinetics are dependent on enzyme concentration. The maximum observed value for G′, 713 Pa, corresponds to a bulk elastic modulus, E, of 2.1 kPa, which is comparable to literature values for PEG-based synthetic ECM gels (Fairbanks et al., Adv. Mater. 21, 5005-5010, 2009; Zustiak et al., Acta Biomaterialia 6, 3404-3414, 2010). The present assay used SrtA-3M at substrate:enzyme ratios of 3:1 and 7:1, hence each SrtA enzyme variant would likely act primarily locally and gelation may have been limited by enzyme kinetics. Regimes that force enzyme diffusion for complete reaction will be examined.

Next, the viability of a human mesenchymal stem cell (MSC) line encapsulated in gels formed by a well-established Michael-type addition reaction of acrylate/thiol macromers was compared to the viability of MSCs encapsulated in gels formed by SrtA-3M-mediated crosslinking, using 5% (w/w) macromer gels. Macromers for the acrylate/thiol gel were 10K 8-arm PEG acrylate with a 5K 4-arm PEG-thiol. To mitigate loss of viability in gels lacking adhesion ligands, gels were formed on top of MSCs attached to the bottom of a 96-well imaging plate (Ibidi). Four hours after plating, the culture medium was removed and 10 μL hydrogel precursor solution (or PBS control) was added. After 5 minutes at 37° C., 60 μl of medium (10% serum) was added to each well (N=3). Cells were incubated for 24 hr and stained for viability (FIG. 2). Cells cultured under the Michael-type and SrtA-3M crosslinked gels showed comparable viability (˜82%), while cells cultured with no hydrogel were 94% viable. The slightly lower viability under gels compared to PBS may reflect removal of dead cells during post-stain washing steps in PBS, or transient serum deprivation for cells cultured under the gel. MSC appear to have a high tolerance for SrtA-3M, as it was present at ˜50 μM during culture.

### Example 2

### Hydrogel dissolution Using SrtA

A. SrtA combined with a small peptide substrate leads to rapid gel breakdown.

Using gels formed by crosslinking 5 uL of precursor solution comprising 5 wt % macromer (338 μM SrtA-3M) in Eppendorf tubes in HEPES pH 7.4 with 10 mM CaCl2, reversibility was evaluated by adding 15 μl of one of the following at time zero: i) buffer; (ii) buffer+SrtA-3M (338 μM); or (iii) buffer+SrtA-3M (338 μM)+GGG (990 μM). The time of hydrogel dissolution was determined as the time at which the entire 20 μl volume could be pipetted up and down (n=2). In the presence of SrtA-3M and soluble GGG, the gel was broken down in 11 minutes, while the gel with SrtA-3M broke down in ˜2.5 hours. The hydrogel to which only buffer was added was stable after 4 hrs; SrtA was not washed out of this gel post-formation and was present at an average concentration of 85 μM. Thus, addition of soluble GGG substrate dramatically enhances the breakdown of the gel, pushing the kinetics within the range of interest for analysis of cell signaling.

B. Further Characterization of Sortase-Mediated Hydrolytic Degradation

Sortase-mediated hydrogel degradation in the presence of soluble GGG was visualized. Briefly, PEG-norbornene (Mw 20k, 8-arm) gels crosslinked with the peptide sequence GCRD-LPRTGGPQGIWGQ-DRCG, 30 μM GCRDRGDSP-fluorescein for visualization was conjugated to the gels, formed in a syringe (17 μl, disk of 2.35 mm radius and ˜1 mm height before swelling). Gels were soaked for 24 hours in DMEM+10% FBS. Gels were then transferred into an Eppendorf tube and sortase (pentamutant—SrtA-5M) solution (in FBS media) was added for 30 minutes at 37° C. GGG was added to a final concentration of 18 μM, and final concentration of sortase was 50 μM. Gels were placed in a tube shaker (400 rpm) at 37 ° C. Gels were visualized under blue light until the fluorescent signal was homogeneous in the solution—time at which the gel has completely degraded. The time line of the assay from beginning to end is illustrated in FIG. 14. FIG. 15 shows preliminary studies demonstrating that gel dissolution in the absence of GGG is very slow even in the presence of high (416 μM) or low (250 μM) sortase concentration (using either Srt-3M—triple mutant—or Srt-5M—pentamutant). FIGS. 23-26 herein provide additional gel dissolution data as measured by release of macromers; these additional data show faster dissolution time that is more amenable to studies in, e.g., cell signaling.

The dissolution rates may depend on properties of the enzyme, the reaction product driving the reverse reaction, gel dimensions, and/or the timing of steps in the protocol, as well as how these parameters influence cell functions. One advantage of the SrtA-mediated approach is the two factors used for dissolution, SrtA and GGG, which can be introduced in temporally discrete steps, thus potentially improving the kinetics of dissolution and minimizing disruption of cellular behavior. The general strategy is to first saturate the gel with SrtA. SrtA acts only weakly on the gel in absence of GGG, causing minimal perturbation of the cellular microenvironment during this step. Dissolution is then initiated by applying a relatively high external concentration of a small peptide substrate (e.g., GGG) to drive the reverse reaction. An identical strategy can be used to release growth factors such as EGF that are tethered to the gel with an LPRTG sequence in the tether (as exemplified herein).

Small-dimension gels dissolved within 11 minutes using SrtA-3M (KM, GGG=1.8 mM) with GGG concentrations <1 mM. The penetration time for GGG in the center of a 1 mm-thick gel to reach ˜10% of the external concentration, estimating DGGG-gel as ˜5×10−6 cm2/s, is roughly 2 min. Hence an external concentration of 18 mM would be expected to drive dissolution of 1 mm gels pre-saturated with SrtA-3M within 13 min, and if dissolution rates scale with kcat/K M, LPTG, the time could be decreased around 3-fold to ˜4 min. The dissolution data described in the assay above can be used to screen a parameter space comprising the 3 SrtA mutants, GGG, and LPRTG concentrations ranging from 0.1-10 KM, GGG and 0.1-10 KM, LPTG against a panel of gels made by SrtA-mediated crosslinking or by standard norbornene-UV crosslinking, where the norbornene/UV-crosslinked gels is made with macromers containing the LPRTG sequence; in both types of gels the context of the LPRTG sequence may be varied by including protease degradation peptides.

Complementary approaches may be used to assess whether the dissolution process significantly modifies cell behavior. For example, acute loss of viability or changes in morphology (assessed by phalloidin staining of actin) may be detected upon exposure to SrtA and the substrate for the observed dissolution times, using an assay on gel-covered adherent cells similar to that shown in FIG. 2 and cells entirely encapsulated in thin gels (as in FIG. 3). A more sensitive assay that can be carried out in situ as gels are dissolving is examination of intracellular ROS (reactive oxygen species) using visualization of the dye. Measuring intracellular signaling pathways in an unperturbed fashion may be difficult to implement for control conditions if cells are encapsulated, as this requires cell lysis. Thus, changes in intracellular Erk, AKT, and P38 activities may be examined for cells cultured on top of gels, as cells can be fully lysed under control (not exposed) or reagent-exposed time points and lysates can be analyzed directly with direct-activity reagents (Stains, et al. Chemistry & Biology 19, 210-217 (2012)). Culturing on top of gels will serve as a control for how the cell signaling changes as the adhesion sites are diminished in the microenvironment during gel dissolution.

### Example 3

### Functional Studies of Modified PEG Hydrogels

SrtA variants have been widely used for protein modification. The present invention provides methods of using SrtA-mediated coupling to effect, e.g., growth factor and adhesion ligand incorporation into PEG gels, thereby modifying the hydrogel to produce a 3D environment to support tissue morphogenesis in vitro. It has been previously shown that by combining sortase-mediated coupling and tetrazine ligation approaches, two epidermal growth factor (EGF) or two neuregulin-1 (NRG) moieties via PEG tethers can be efficiently linked over a range of PEG tether lengths (Krueger et al., Angew. Chem. Int. Ed. 53, 2662-2666, 2014). This was accomplished by developing high-yield (30-50 mg/L) expression protocols for EGF and NRG containing the relevant SrtA substrate motifs linked to the C- or N-terminus. Indeed, virtually any biomolecule may be incorporated (“tethered” and “functionalized”) into the hydrogel using the present methods. FIG. 16 shows a generalized schematic of sortase grafting EGF or Neuregulin to PEG hydrogels. Here, C-LPRTG-fam (GCRE-LPRTGGGK(fluorescein)- NH₂), C-LPRTG (LPRTGGGK- NH₂), or GGG-C (GGGTTSS-ERCG- NH₂) is mixed with PEG maleimide (Mw 10k, 20k, 40k) and PEG thiol and polymerized to form a gel. Sortase and GGG-EGF (or GGG-Neuregulin) are subsequently added to tether EGF (or Neuregulin) to the gel. As FIG. 16 shows, G-fam, which is quantifiable, is released as a result of the sortase-mediated reaction.

A. Modified PEG hydrogels support survival and remodeling by human endometrial cells and iPS-derived endothelial cells.

Thus, the methods of forming and dissolving gels according to the present invention will be used to support 3D tissue morphogenesis in vitro. Complementary morphogenesis assays may be used in order to assess properties of gels different cell types, as illustrated in FIGS. 3A, 3B and 3C, where established methods were used to encapsulate cells in functionalized PEG hydrogels. Representative images of the Ishikawa human endometrial epithelial cell line show polarization behaviors of cells cultured in gels formed via crosslinking 8-arm 40 kDa PEG vinyl sulfone with an MMP-1 degradable crosslinker, CRDGPQGIAGQDRC (FIGS. 3A, 3B and 3C). PEG macromers were pre-functionalized to give a final ligand concentration of 250 μM. Cells were cultured for 7 days to form cysts, then stained with phalloidin to highlight actin, and assessed for proper polarization (apical actin) or dysregulated polarization (diffuse or basal actin). It has been shown previously that a small branched peptide containing both the PHSRN and RGD motifs (“Syn-K-RGD”), needed for interaction with integrin α₅β₁, enhances cell adhesion and function compared to RGD, which interacts primarily with αᵥβ₃. Interestingly, Ishikawa cells polarize properly in the presence of Syn-K-RGD (FIG. 3A, left image), but polarization is dysregulated with canonical RGD (FIG. 3A, right image). FIG. 3B shows the results of a similar peptide screen for morphogenesis of human iPS-derived endothelial cells in PEG gels. A panel of integrin ligands and protease cleavage sites were used. The image shown is a confocal of cells fixed on day 3, phalloidin-stained for actin, and color coded to show interconnected structures that span 300 μm in the z direction. The human MSC cell line, which recognizes RGD, forms networks in PEG gels functionalized with RGD (FIG. 3C). Gels in FIGS. 3B and 3C were crosslinked with norbornene groups via UV activation.

B. Optimizing Complementary Functional Assays

Cells require adhesion ligands and proteolytic remodeling sites for function in gels. Although the minimal peptide sequence RGD can engender adhesion of many cultured cell lines, primary cells often have more complex requirements involving protein domains (Mehta et al., Biomaterials 31, 4657-4671, 2010), and as shown in FIG. 3A, epithelial cell lines exhibit more physiological polarization in response to the SYN-K-RGD peptide compared to RGD. Hence for generality SYN-K-RGD will be employed as a representative adhesion sequence, and will include the full FN9-10 domain in some studies as a comparator large ligand needed for primary cells. Peptide degradation motifs will be included as domains flanking the SrtA ligation motifs to form a section of the crosslinks. Two motifs will be assessed, which may have differential effects on biological and crosslinking responses: (i) a well-established GGPQGIAGQ motif cleaved by MMP2, 7, and 8, employed in the assay for epithelial polarization shown in FIG. 3A and (ii) an alternate motif GCREGPLGMRGERCG which is cleaved effectively by MMP2, 9, 13 and 14, which have previously been used for assessing protease activity (Miller et al., PNAS 110, E2074-E2083, 2013; Marcantonio et al., Biomaterials 30, 4629-4638, 2009; Miller et al., Integr Biol (Camb) 3, 422-438, 2011) as well as in the assay shown in FIG. 3B. To demonstrate that the approach is generalizable to include multiple ligands, EGF will be incorporated using reagents described herein. EGF receptor is polarized in epithelial cells and inclusion of tethered EGF may enhance epithelial function, MSC survival, and angiogenesis.

Different cell types may exhibit different sensitivities to the crosslinking process, thus two general phenotypic assays will be used on a routine basis: tube-forming capabilities of iPS-derived human endothelial cells (FIG. 3B) and survival and polarization of the human endometrial Ishikawa cell line (FIG. 3A). The survival and function of primary human endometrial epithelial cells in co-culture with endometrial stromal cells will also be investigated as representative of fragile primary cells, which may be more susceptible to damage by crosslinking conditions. Isolation and culture of cells from endometrial biopsies may be performed using standard protocol.

C. Optimizing relationships between gel formation variables and resulting gel functional properties for gels formed or functionalized by SrtA-mediated crosslinking

Design principles outlining the structure-function relationship of SrtA-crosslinked hydrogels may be further fine-tuned, and compared to well-established theory for end-crosslinked PEG systems (Lutolf and Hubbell, Biomacromolecules 4, 713-722, 2003). Commercially-available (Jenken) 8-arm PEG macromers have been grafted through a Michael-type reaction to cysteine-conjugated, N-terminal GGG or LPXTG SrtA recognition sites. Additional studies will be carried out using SrtA mutants over ranges of enzyme:substrate of 1:3 to 1:50, using previously-described high-yield expression methods for expression of mutants (Krueger, et al., Angew. Chem. Int. Ed. 53, 2662-2666, 2014). To access a wide range of bulk gel properties, the polymer sol concentration (2.5-10% total polymer), the stoichiometries of grafted GGG and LPXTG ligands (0.5:1 to 1:1.5), and the 8 arm PEG macromer molecular weight (10k and 40k) may be varied. These parameters are expected to generate gels that span the physiologically relevant range of mechanical properties and pore size, as well as ligand “tether” mobility during crosslinking. Oscillatory shear rheometry and AFM indentation will characterize the time to gelation and bulk properties of the resultant gels, and swelling ratios will be characterized (Griffith and Lopina, Biomaterials 19, 979-986, 1998; Williams et al., Tissue Eng Part A 17, 1055-1068, 2011; Peyton et al., Biotechnol. Bioeng. 108, 1181-1193, 2011; Oelker, et al., Soft Matter 8, 10887, 2012). Fluorescence recovery after photobleaching, (FRAP) an established technique (Oelker, et al., Soft Matter 8, 10887, 2012) will be used to assess diffusivity of proteins in hydrogels, employing GFP as a model for SrtA due to their comparable Mw and dimensions; ovalbumin (45 kDa); and IgG (150 kDa). A limited set of investigation of the effects of 10% serum on crosslinking will be carried out, as it may help cells survive post-crosslinking. It is expected that sortase allows minimal non-specific protein incorporation in the gels formed by sortase crosslinking.

The competing processes of crosslinking and hydrolysis can be more fully characterized by monitoring the rate and extent of the forward reaction by quantifying the release of a fluorescein-labelled carboxy termini of the LPRTG peptide; i.e., the product of the ligation reaction between GGG-(PEG) and (PEG)-LPRTGGK(fluorescein), is soluble GGK(fluorescein) which diffuses out of the gel and can be quantified. The extent of forward reaction is not necessarily indicative of the total crosslinking, but when coupled with mechanical measurements, gives a comprehensive metric for monitoring and optimizing the reaction of SrtA-crosslinked systems. The rate of forward reaction to the aggregate degree of crosslinking can be compared, as monitored by real-time mechanical measurements and extent of swelling. Gel stability up to 5 days post-crosslinking can also be characterized, using a combination of swelling and protein diffusion studies to monitor integrity of crosslinks. Gel stability may be affected by residual SrtA (although preliminary data herein suggests this effect is modest for SrtA-3M); with a characteristic diffusion time of τ_{D}˜L²/(4D_{SrtA-gel}) and values of SrtA diffusion coefficients in the gels in the range 10⁻⁷-10⁻⁶ , cm²/s (Sperinde and Griffith, Macromolecules 33, 5476-5480, 2000), SrtA may require hours or days to wash out of a 1 mm thick gel completely, the time scale for which can be determined.

Gelation kinetics and properties with protease-sensitive peptides and cell adhesion and growth factor motifs incorporated into the gels may be further characterized. Using a “one-pot” gelation approach, PEG macromers (fully functionalized with SrtA ligation motifs), and bioactive adhesion and growth factor ligands (also modified with GGG- or -LPRTG) are added in stoichiometric amounts ranging from 1:8 to 1:3 (total ligand: total PEG chain ends). With this approach, complete consumption of all PEG chain ends requires stoichiometrically-matched amounts of ligands with complementary SrtA substrate motifs; this can be an advantage when it is desired to tether two ligands (e.g., adhesion and growth factor). Further, cell responses to the gelation procedures for gelation processes with kinetics in a range of <10 minutes will be characterized. Initial tests involve simple viability assays 2 hr post-encapsulation. For gels that preserve cell viability (e.g., where viability is >80% and comparable to standard norbornene-crosslinked control), phenotypic assays were carried out, with cells observed daily for up to 2 weeks.

### Example 4

### SrtA-Mediated Dissolution of Synthetic Extracellular Matrix for Studying Cell-Cell Communication

The studies herein demonstrate the feasibility and advantages of using transpeptidase-sensitive sequence-containing hydrogels for examining cell-cell communication and signaling. As shown herein, gels crosslinked by various methods can be dissolved rapidly by SrtA in the presence of soluble GGG peptide, when the crosslink contains LPRTG. The dissolution process does not negatively affect cell viability. Due to the low extracellular abundance of C-terminal sequences that can serve as substrates for sortase (e.g., LPXTG), the present method minimizes reaction with native proteins.

Materials and Methods

A. Hydrogel Fabrication

Hydrogels were fabricated using norbornene/thiol-ene click chemistry or Michael addition chemistry inside a 1 mL syringe (Becton, Dickson and Company, REF 309659) modified by cutting of the tip at the 0.1 mL mark syringe.

PEG norbornene (PEG-NB) hydrogels were crosslinked by UV irradiating 18 uL of a solution containing 4 wt % 8-arm PEG-NB (M_{W} 20,000, JenKem Technology, Beijing), an MMP- and SrtA-sensitive dithiol-terminated peptide [(Ac)GCRDLPRTGGPQGIWGQDRCG(Am), Boston Open Labs, Cambridge)] at a stoichiometric ratio (r) of 0.55 thiols per norbornene (unless otherwise indicated), the cell attachment peptide (Ac)PHSRNGGGK-(Ac)GGGERCG-GGRGDSPY(Am) (Syn-K-RGD) (Boston Open Labs, Cambridge, Mass.) at 500 μM nominal concentration (unless otherwise indicated), and IRGACURE 2959 (0.05 wt %) (Ciba, Prod. No. 0298913AB). For quantification of macromer release during gel dissolution, fluorescein-labeled SynKRGD [(Ac)PHSRNGGGK-(fluorescein)GGGERCG-GGRGDSPY(Am) (F-Syn-K-RGD) (Boston Open Labs, Cambridge, Mass.)] was substituted for 14% of the total attachment peptide. The precursor solution was UV-irradiated for 5 seconds at ˜800mW/cm² and resulting hydrogels were placed in a 24-well plate with 600 μL DMEM/F12/FBS per well and allowed to swell in a humidified atmosphere at 37° C. and 5% CO₂ to emulate culture conditions. Cell encapsulation followed similar procedures (described below).

PEG vinyl sulfone (PEG-VS) hydrogels were fabricated using peptide-functionalized macromers (where peptides refer to adhesion peptides) prepared by reacting a 7.2 mM solution of 8-arm PEG-VS [M_{W} 40,000 Da ; JenKem Technology, Beijing] with free thiols (—SH) on adhesion peptides (Syn-K-RGD) in 1× PBS with 1 M HEPES (pH 7.8) for 30 minutes. Immediately after the functionalization reaction, the peptide-functionalized PEG-VS (fPEG-VS) macromer (average 6.7 free -VS groups per macromer) solution was diluted in PBS to 5 wt %. Peptide-functionalized 8-arm PEG-VS macromers were then reacted with the cysteine thiol (-SH) groups on the bifunctional sortase and MMP sensitive peptide crosslinker (Ac)GCRD-LPRTG-GPQGIWGQ-DRCG(Am) (SM-CL_{W}) in volumetric ratios of 4.9:8.6:1 fPEG-VS:cells:SM-CL_{W} to yield a final crosslinking solution with nominal (before swelling) composition of 0.5 mM Syn-K-RGD peptide and 1.2 mM total PEG macromers (5 wt %) in PBS, 1 M HEPES buffer (pH 7.8). For quantification of macromer release during gel dissolution characterization experiments, the 14% of the adhesion peptide was F-Syn-K-RGD.

Srt-A crosslinked PEG hydrogels (PEG-SrtA) were fabricated using stoichiometrically-balanced ratios of two complementary macromers prepared by reacting a 7.2 mM solution of 8-arm PEG-VS (M_{W} 40,000 Da) with free thiols (—SH) on SrtA substrates -CLPRTG or GGGC- (1:1.2 VS:peptide ratio) in 1× PBS with 1 M HEPES (pH 7.8) for at least 30 minutes. Product was dialyzed for 3 days (4° C.), frozen (−80° C.) and lyophilized. Initial characterization of the SrtA-mediated gelation process was carried out by mixing macromers (each 2.5 wt % or 650 uM) in a HEPES buffer (pH 7.4) with CaCl₂ (10 mM) with SrtA (final concentration135 uM or 338 uM) and conducting oscillatory shear measurements (0.5 Hz with strain=1%) on TA Instrument AR2000 rheometer. Details of conditions for cell encapsulation are given below.

B. Hydrogel Dissolution Quantification

PEG-NB or PEG-VS hydrogels (18 μL) were synthesized as described above and allowed to swell in a humidified atmosphere at 37° C. and 5% CO₂ for 24 hours in DMEM/F12/FBS. Swollen gels (˜25 μL swollen volume) were removed from the media using a spatula and transferred into a 1.6 mL Eppendorf tube. A 60 μL solution of sortase A penta-mutant P94R/D160N/D165A/K190E/K196T (SrtA), expressed and purified as previously reported (Liu, Krueger), and Gly-Gly-Gly (GGG) (Sigma-Aldrich) in DMEM/F12/FBS was added to the hydrogel in the Eppendorf tube at 50 μM and 18 mM respectively unless otherwise specified. Where indicated, srtA was added for 10 minutes or 30 minutes and incubated at 37° C. prior to adding GGG. Upon addition of both srtA and GGG, the tubes were placed on a thermal shaker and mixed at 300 RPM during gel dissolution. At each of the time points indicated in the plots, 2 μL were removed from the gel-containing tubes and added to 38 μL of 50 μM HEPES buffer in a 384-well plate. Fluorescence (λₑₓ=485 nm, λₑₘ=525 nm) of each time point sample was measured using a microplate reader (SpectraMax M2^{e}, Molecular Devices). A fluorescein linear standard curve containing 0, 20, 50, 100, or 250 μM was established to ensure the fluorescence measurements for each time point were in a linear range.

C. Cell Maintenance

Human mesenchymal stem cells immortalized with h-tert (tHMSC) were routinely cultured in a humidified atmosphere at 37° C. and 5% CO₂ in Dulbecco's modified Eagle's medium (DMEM) containing 10% fetal bovine serum (FBS; Atlanta Biologics), 1% L-glutamine, 1% non-essential amino acids, 1% sodium pyruvate and 1% penicillin/streptomycin (Gibco). (Alverez et al., JBC 286(31): 27729-40, 2011). Ishikawa human endometrial adenocarcinoma cells (Sigma-Aldrich){Nishida, Lessey 1996} and htert-immortalized human endometrial stromal cells (tHESCs) (ATCC) were routinely cultured in a humidified atmosphere at 37° C. and 5% CO₂ in phenol red free DMEM/F12 (mixture of Dulbecco's Modified Eagle's Medium and Ham's F-12 (Gibco) media supplemented with 1% penicillin/streptomycin (Gibco) and 10% v/v dextran/charcoal treated fetal bovine serum (Atlanta Biologicals) (DMEM/F12/FBS), replacing medium every 2-3 days. Cells were split 1:4 (Ishikawa) or 1:2 (tHESCs) once they reached 70-80% confluency and used prior to passage 10. Following reports of possible contamination of endometrial cell lines by HeLa or other lines, STR profiling analysis (Genetic Resources Core Facility, Johns Hopkins School of Medicine, Institute of Genetic Medicine) confirmed the fidelity of the tHESCs and Ishikawa cell lines.

Human colorectal adenocarcinoma SW48 cells (ATTC, CCL-231), and human colorectal adenocarcinoma SW48 G12D mutant cells (Horizon, HD 103-011) were routinely cultured in a humidified atmosphere at 37° C. and 5% CO2 in RPMFFBS (Roswell Park Memorial Institute medium (Gibco) and 10% v/v fetal bovine serum (Atlanta Biologicals), with medium replacement every 2 days. SW48 WT and SW48 G12D mutant cells were split 1:10 once they reached 70-80% confluency, and routinely used prior to passage 10.

D. Cell Encapsulation in SrtA-Crosslinked Gels

In pilot experiments, tHMSC adherent to 96-well angiogenesis plates (Ibidi) were overlaid by a SrtA-crosslinked gel. Four hr post plating, medium was replaced by PBS or by 10 uL of gel precursor solution comprising 5 wt % peptide macromers (1:1 mix of 8-arm PEG-C-LPRTG and PEG-GGG) in PBS containing Ca++ and Mg++ with 338 μM triple mutant sortase. After 5 min, 60 uL of medium containing 10% FBS was added to each well (N=3). Cells were incubated for 24 hours. Parallel control cultures were overlaid with a PEG-VS gel formed by Michael-type addition of 5 wt % total polymer 1:1 tiol:VS (10 kDa 8-arm PEG acrylate/5 kDa 4-arm PEG-thiol).

E. 3D stromal and epithelial co-culture cell encapsulation in PEG-VS hydrogels

Hydrogel precursors were combined with cells and polymerization was carried out in 1-mL modified syringes using the PEG-VS fabrication protocol described above, with modification of macromer composition where indicated. Peptide-functionalized PEG-VS (fPEG-VS) macromer (average 6.7 free—VS groups per macromer) solution was mixed with a cell suspension of 1:1 stromal and epithelial cells (13.49×10⁶ cells/mL in PBS). Peptide-functionalized 8-arm PEG-VS macromers were then reacted with the cysteine thiol (-SH) groups on the bifunctional sortase and MMP sensitive peptide crosslinker SM-CL in volumetric ratios of 4.9:8.6:1 fPEG-VS:cells:SM-CL to yield a final crosslinking solution comprising 8×10⁶ cell/mL (200,000 cells in 25 uL), 2 mM total adhesion peptide, 1.2 mM total PEG macromers (5 wt %), and 2.5 mM crosslinking peptide in PBS, 1 M HEPES buffer (pH 7.8). Nominal adhesive peptide concentrations in the final gel was 2 mM Syn-K-RGD. In separate experiments, matrix binding peptides were included to stabilize an epithelial layer formed on top of the hydrogel. After pipetting the mixture up and down for 2 minutes, 25 μL was pipetted onto each syringe. The solutions were allowed to gel (˜6 additional minutes) and were incubated at RT for 15 minutes to allow crosslinking to proceed to completion. After gelation was complete, the gels were moved to 24 well plates and 400 μL of DMEM/F12/FBS was added to each gel. Cultures were maintained in a humidified incubator at 37 ° C., 95% air, 5% CO₂.

F. 3D epithelial cell encapsulation for acinar structure 3D passaging

Hydrogels were fabricated on the top membrane of Transwell inserts (Corning #3470, 6.5 mm diameter, 0.4 um pores, 0.33 cm² culture area) using Michael-type reaction chemistry. Briefly, peptide-functionalized macromers were prepared by reacting 8-arm PEG-VS (1.4 mM) with free thiols (-SH) on adhesion peptides in 1× PBS with 1 M HEPES (pH 7.8) for 30 minutes. Immediately after the functionalization reaction, the peptide-functionalized PEG-VS (fPEG-VS) macromer (average 6.7 free—VS groups per macromer) solution was mixed with a Ishikawa cell suspension (8.2×10⁷ cells/mL in PBS). Peptide-functionalized 8-arm PEG-VS macromers were then reacted with the cysteine thiol (—SH) groups on the bifunctional sortase sensitive and MMP-degradable peptide (SM-CL) in volumetric ratios of 8.6:1:0.4 fPEG-VS:cells:MMP-CL to yield a final crosslinking solution comprising 8×10⁶ cell/mL (96,000 cells in 12 μL), 2 mM total adhesion/matrix binding peptide, 1.2 mM total PEG macromers (5 wt %), and 1.9 mM crosslinking peptide in PBS, 1 M HEPES buffer (pH 7.8). Nominal adhesive peptide concentration in the final gel was 2 mM Syn-K-RGD. After pipetting the mixture up and down for 2 minutes, 12 uL/insert was pipetted onto Transwell inserts (12 μL/insert) contained in 24 well plates. After the gelation process (gelation occurred at ˜7 minutes), plates were incubated at RT for 15 minutes to allow crosslinking to proceed to completion. After gelation was complete, DMEM/F12/FBS was added to the apical (100 uL) and basolateral (600 μL) sides of the Transwell. Cultures were maintained in a humidified incubator at 37° C., 95% air, 5% CO₂.

G. ERK and MAPK Phosphosignaling Response to SrtA and GGG

200,000 per well SW48 wild type and KRAS G12D mutant cells were seeded in 6-well plates in 3 mL per well of RPMI1640 10% FBS. Cells were cultured for 4 days in a humidified incubator at 37° C., 95% air, 5% CO₂ with a medium change at day 2. At day 4, 100 μL of concentrated srtA and/or GGG solution was added to the culture medium to yield final concentrations of 0, 4.5, 9, or 19 mM GGG, 0 or 50 uM srtA, 50 μM srtA+18 mM GGG, or a buffer control (50 mM HEPES, 150 mM NaCl, 10 mM CaCl2, pH 7.9), and incubated at 37° C. for 30 minutes. The 24-well plate was then placed on ice and the media and treatment solutions were aspirated. RIPA buffer (100 μL) (Sigma-Aldrich, R0278) was then immediately added to each well to lyse the cells. After less than 5 minutes, the lysates were transferred into Eppendorf tubes and frozen at −80° C. Western blot indicates that SrtA and GGG combinations have no effect on ERK and MET phosphorylation signaling (FIG. 22).

H. Determination of Soluble Cytokine Depletion by SrtA, Trypsin, and Liberase™ Using Luminex

A solution of 27 cytokines of known concentrations (67 μL), recombinant standards from a Bio-Plex Pro human cytokine 27-plex assay (Bio-Rad, #M500KCAF0Y) were incubated with 14 μL of SrtA (10, 30, or 50 μM final), GGG (9 or18 mM final), srtA+GGG (50 μM+18 mM, or 30 μM+9 mM final), trypsin (1X=0.25% final) (Gibco, Ref 15090-046), Liberase™ (10 μg/mL final) (Roche, Ref 05401119001), or a buffer control (50 mM HEPES, 150 mM NaCl, 10 mM CaCl2, pH 7.9). After a 45 mins incubation, 8.1 μL of protease inhibitor cocktail (Roche, Prod. No. 05892953001) were added to all conditions for a final concentration of 5 mg/mL, as recommended by the vendor. The cytokine concentrations after treatment were measured by Luminex assay as described below. Data reported as percent decrease compared to the buffer control.

I. IL1-b Stimulation of Endometrial Epithelial/Stromal Co-Cultures

1. Multiplex Measurement of Protein Concentrations Inside Hydrogel and in Culture Media of 3D Epithelial/Stromal Co-Culture

Epithelial and stromal cell co-cultures were encapsulated in PEG-VS as described above in 25 μL hydrogels submerged in 400 μL of DMEM/F12/FBS. Blank PEG-VS gels (hydrogels of the same exact composition but with no cells) were fabricated at the same time and submerged in 400 μL of 50 mM HEPES, 150 mM NaCl, 10 mM CaCl2, pH 7.9. At the time points indicated, the co-culture and blank gels were removed from the culture media, transferred into Eppendorf tubes, and their weight was recorded to estimate their volume for future dilution correction of cytokine concentrations (swollen gels were ˜60 μL). Co-culture gels were dissolved in 90 μL of srtA and GGG at 50 μM and 18 mM final concentrations (accounting for gel volume), respectively at 37° C. in 50 mM HEPES, 150 mM NaCl, 10 mM CaCl₂. To favor homogeneous dissolution, hydrogels were infused with 76.5 μL srtA for 10 minutes at 37° C. prior to adding GGG (13.5 ηL). Simultaneously, 60 μL of culture media from each co-culture gel were added to blank gels, and then 30 μL of srtA and GGG at 50 μM and 18 mM final concentrations (accounting for gel volume) respectively were added at 37° C. in 50 mM HEPES, 150 mM NaCl, 10 mM CaCl₂. Co-culture gels and their respective media were diluted equivalently in the dissolution process. Dissolution was allowed to take place on a thermal shaker with gentle mixing at 300 RPM. Upon gel dissolution (8-10 minutes), the dissolved-gel solutions/cell suspensions were spun down for 3.5 minutes at 350 RCFs and the supernatant for each sample was transferred into a new tube to remove the cells prior to soluble cytokine measurements. 10 μL of protease inhibitor cocktail (Roche, Prod. No. 05892953001) were added to all conditions for a final concentration of 5 mg/mL, as recommended by the vendor prior to Luminex assay cytokine quantification.

2. Cytokine Luminex Assay

Cytokines from dissolved co-culture gels, co-culture gel media with dissolved blank gels, and cytokine solutions of known concentrations incubated with the enzymes described above, were all measured by Luminex assay (BioRad). Protocols provided by the manufacturer were adapted to allow the assay to be performed in a 384 well plate to avoid introducing batch effects. Ten-point standard curves plus blanks (DMEM/F12/FBS never exposed to cells) were included for quantification.

For each cytokine, 5-parameter logistic curves were fit to the standards, including the blanks (Cardillo G. (2012) Five parameters logistic regression—There and back again, <<www.mathworks.com/matlabcentral/fileexchange/38043>>). Curves were used to calculate concentrations for each sample replicate. Median fluorescence intensities (MFI) for the samples below the lower asymptote or above the upper asymptote of the standard curve were imputed to be either the MFI of the minimum asymptote or 99% of the MFI of the maximum asymptote, respectively. Reported values are the mean of 3 technical replicates and 3 biological replicates minus concentrations of no cell media controls.

3. DNA Synthesis Assay on Epithelial Acini Cultures

Click-iT EdU Alexa Fluor 488 kit (Life Technologies) was used to quantify cells actively synthesizing DNA. On days 7, 9, or 12 of culture, hydrogels were incubated with 10 μM of 5-ethynyl-2′-deoxyuridine (EdU) for 4 h at 37 ° C., 95% air, 5% CO₂. Cells were fixed with 3.7% formaldehyde in PBS for 15 min at RT. Cells were washed twice with 3% BSA in PBS and permeabilized with 0.5% Triton X-100 in PBS for 20 min at RT. Click-iT reaction cocktail was prepared as described by the manufacturer and 100 μL per well was added. Cells were incubated for 30 min at RT protected from light. After cells were washed once with 3% BSA in PBS and once with PBS, they were incubated with Hoechst 33342 diluted 1:2000 in PBS for 30 min at RT protected from light. Cells were washed twice with PBS and imaged using a Leica DMI 6000 microscope and Oasis Surveyor software. Cell nuclei were counted using ImageJ64 software. The percentage of cells synthesizing DNA was computed as the ratio of EdU positive cells divided by the total number of Hoechst counter-stained cells.

Results

A. SrtA Crosslinks Peptide-Modified PEG in a Cell-Friendly Manner to Form a Synthetic ECM

The versatility of SrtA to catalyze protein-PEG conjugates, and to link proteins to the surfaces of cells engineered to express SrtA substrates, motivated us to test whether SrtA could be used to form crosslinked PEG hydrogels in a cell-friendly manner. SrtA crosslinking is appealing, as the SrtA substrates are extremely rare in mammalian proteins, hence SrtA is unlikely to crosslink cells to the gel or alter cell surface proteins. We first established that PEG macromers undergo SrtA-mediated crosslinking by using the SrtA triple mutant to crosslink complementary 8-arm PEG macromers modified with -CLPRTG or GGGC-, using oscillatory shear measurements to characterize gel formation. We observed a rapid rise in storage modulus G′, which reached a plateau of 713 Pa (E=2.1 kPa, comparable to literature values for PEG gels) in under 4 min when using a high concentration of SrtA (338 uM) and within 20 min at more modest concentrations (135 uM). In a pilot experiment, we observed that the viability of culture-adherent htMSC line encapsulated in PEG gels formed by SrtA was comparable to that of MSCs in gels formed by standard Michael-type addition 24 hr after encapsulation (82% viability for both conditions, comparable to literature values for Michael-type encapsulation). SrtA appears to have minimal effects on cultured MSCs, as it was present at ˜50 uM during culture.

As a more stringent test of using SrtA-mediated crosslinking to create a synthetic ECM for 3D culture, we evaluated behavior of endometrial and stromal epithelial cells co-cultured in SrtA-crosslinked PEG gels containing the RGDS adhesion and an MMP-sensitive crosslinker in comparison to PEG gels formed by well-established Michael-type addition (Lutolf and Hubbell). Epithelial cells proliferated and formed acinar structures (FIG. 20A, 32, and 33) in synthetic ECM gels, and production of a characteristic endometrial functional marker, IGFBP-1, was indistinguishable between the two crosslinking systems (FIG. 20B). This suggests that SrtA crosslinking is suitable for complex 3D cultures, offering the advantage of the enzyme bio-orgthogonality and potentially allowing cell encapsulation to be done in the presence of serum proteins without non-specific covalent incorporation into the gel in a one-pot synthesis.

B. Synthetic ECM is Rapidly Dissolved by SrtA-Mediated Transpeptidation, Releasing Intact Epithelial Acinar Structures

The transpeptidase activity of SrtA can be a drawback in the context of protein ligation reactions, as desirable product can be further modified in the presence of N-terminal glycine substrates. We speculated that this behavior could be exploited, however, to dissolve synthetic ECM hydrogels crosslinked with an LPRTG sequence, as addition of SrtA together with soluble GGG drives a transpeptidase reaction that functionally severs the crosslink (Cambria et al., Biomacromolecules, 16(8): 2316-26, 2015, incorporated by reference herein in its entirety). In order to establish kinetics of the dissolution process, as well as to determine whether rapid dissolution (e.g., within minutes) could be accomplished for a range of enzyme, substrate, and gel crosslinking parameter values, we synthesized gels incorporating fluorescently-tagged versions of the adhesive peptide Syn-K-RGD to monitor macromer release as a measure of gel degradation (e.g., FIGS. 23A-23D, 24 and 25).

As a benchmark, we first tested dissolution of relatively large gels (discs 1 mm thick with 4.7 mm diameter post-swelling) using a concentration of SrtA at the upper end of the values reported for cell surface labeling (50 uM) and a concentration of soluble GGG of 18 mM, which is approximately 5-fold above the SrtA-5M Kₘ for the N-terminal glycine substrate (K_{M, GGG}=2.9 mM (Liu et al., PNAS 108(28): 11399-11404). This protocol resulted in complete gel dissolution in 14-17 min (FIG. 23B), and the gel appeared to dissolve from the surface (i.e., to shrink in dimensions as the process proceeded). SrtA (M_{w}=23,000) diffuses more slowly than GGG (Mw=235) and is catalytically required for the cleavage, hence the dissolution with the initial protocol is likely limited by the time required for SrtA to penetrate the gel fully. Defining the effective penetration time for GGG as the time required for the GGG concentration to reach ˜2× Km (˜6 mM)—i.e., a concentration that drives the reaction at near the maximum rate—it is possible to shorten the effective penetration time of GGG by increasing the concentration external to the gel.

We therefore postulated that rapid, homogeneous gel dissolution could be accomplished by a two-step process: addition of SrtA first followed by addition of GGG at a relatively high external concentration of GGG. Indeed, addition of SrtA for 30 minutes prior to addition of GGG (final 50 μM SrtA and 18 mM GGG) resulted in gel dissolution at ˜5 minutes upon addition of GGG (FIG. 23B), with dissolution appearing to occur as a bulk breakdown rather than surface erosion. A modest detectable release of PEG macromer was observed during the SrtA incubation step as seen in minute one and zero in FIGS. 23B and 23C, respectively, presumably due to the known ability of SrtA to catalyze hydrolysis in the absence of an N-terminal glycine. Because we conducted the dissolution experiments in the presence of medium containing 10% serum, an alternate mechanism for the low level of SrtA-mediated reaction in the absence of GGG is the presence of N-terminal glycines in the serum; peptides containing N-terminal glycines can arise in serum from proteolytic degradation of hormones such as GNRH, and glycine itself has been shown to be present at 0.7 mM in serum. However, gel dissolution times were similar in serum-containing and serum-free media, hence the presence of serum does not affect dissolution times. The dissolution kinetics can also be modulated by the concentration of SrtA (e.g., FIG. 23C and FIG. 24), and are modestly influenced by crosslinking properties, including crosslink density and macromer properties (FIGS. 25 and 26), though relatively unaffected by the particular type of crosslinking chemistry used to make the gel (FIG. 25, upper panel). Altogether these data suggest that the dissolution method is robust to a wide variety of hydrogel properties and that it is easily transferable to other hydrogel systems.

With a suitable dissolution protocol established (FIGS. 20A and 23A), we next used SrtA-mediated dissolution to release co-cultures of endometrial stromal and epithelial cells encapsulated in synthetic ECM created by either SrtA-mediated crosslinking or standard Michael-type addition (schematic shown in FIG. 20A) and cultured for 6 days.

As expected from the relative lack of SrtA substrates in extracellular mammalian proteins, SrtA-mediated dissolution released intact epithelial acini along with stromal cells (FIGS. 21B and 33). Single cells and epithelial acini replated on TCPS for 3 hr after SrtA-mediated release from synthetic ECM showed strong positive immunostaining for endometrial epithelial (BerEp4) and stromal (CD10) surface markers and epithelial cells retain strong cell-cell junctions (FIG. 21B—Ber-EP4 staining indicated by thick arrow; example of CD10 staining indicated by dashed arrow).

These findings suggest that SrtA-mediated dissolution may provide a novel way to retrieve cells from 3D matrices in a gentle fashion, preserving cell surface protein expression for subsequent analysis or sorting by FACS or other methods, or for subsequent subculture.

C. SrtA-Mediated Gel Dissolution Enables Recovery of Intact Cell-Produced Signaling Molecules, Enabling Multiplex Analysis of temporal Evolution of Local Cell-Cell Communication Networks

Paracrine growth factor and cytokine signaling between stromal and epithelial cells regulates myriad tissue functions but is difficult to parse these extracellular protein networks as they occur in 3D culture. Measurement of molecules in the culture supernate is only partially representative of paracrine networks, due to diffusion barriers for escape of molecules from 3D matrices, and proteolytic degradation of 3D matrices also degrades many extracellular proteins, such that they cannot be quantitatively analyzed by standard immunoassays. We postulated that SrtA dissolution would enable quantitative analysis of growth factors and cytokines in the extracellular environment, and may reveal new features of local communication networks as they occur in real time.

We first compared the effects of the SrtA-mediated gel dissolution protocol to protocols involving standard proteolytic degradation (trypsin and Liberase) on the quantitative recovery of 27 cytokines and growth factors, using a multiplex bead-based immunoassay (Luminex) panel for analysis. Whereas about half the target proteins were undetectable after trypsin or collagenase degradation of gels, the SrtA-mediated dissolution process rendered only IL-15 undetectable (data not shown). This is consistent with the observation that IL-15 is a rare mammalian protein with an LPRTG motif, rendering it susceptible to the SrtA transpeptidase reaction. This finding indicates that SrtA can be used to recover local proteins lacking the substrate motif.

Next, we used SrtA-mediated dissolution to determine whether cytokine accumulations in the local pericellular environment in the synthetic ECM differed significantly from those in the culture supernate. Endometrial epithelial and stromal cells were cultured for 24 hours in the presence of the adhesive peptide Syn-K-RGD. Removing the gels from the culture media and dissolving it allowed for quantification of local hydrogel cytokines. As described in the methods section, in order to avoid any potential artifacts in Luminex detection arising from the presence of PEG macromers, peptides, SrtA, and GGG, the culture media was exposed to a blank PEG hydrogel being dissolved with SrtA and GGG in parallel to the dissolution of the cell-containing gels (FIG. 27 upper panel). FIG. 27, lower panel, reveals significant discrepancies between several of cytokines measured. Notably, at 24 hours, basic FGF and IL-7 were below detectable levels in the culture media, but were at significant concentrations inside the gel where the cells can sense them. IL-10, IL-12p70, and VEGF were at significantly higher concentrations inside the gel compared to the culture media. This data shows that SrtA-mediated dissolution can be used to interrogate the local microenvironment that the cells experience to gain a more accurate representation of it.

D. The SrtA Dissolution Protocol Exerts Negligible Effects on Intracellular Kinase Signaling Pathways

Although SrtA has been used without noticeable effects on viability to label cells that have been engineered to express the SrtA substrates on their surface, the effect of the enzyme on more subtle signaling processes has not been previously investigated. To test the effect of the enzyme and the soluble GGG substrate, SW48 wild type and KRAS G12D mutant cells (on TCPS) were incubated with different combinations of SrtA and GGG for 30 minutes at concentrations comparable to those used previously in the literature (Cambria et al., Biomacromolecules, 16(8): 2316-26, 2015). ERK and MET phosphorylation was qualitatively assessed through Western blot (FIG. 22). Qualitative differences were not observed in kinase phosphorylation between the buffer control and any of the SrtA and GGG combinations. The fact that the SrtA and GGG combinations do not affect these sensitive readouts in cell lines with different basal activation suggests that SrtA/GGG does not dramatically impact signaling processes, while other proteases are likely to influence growth factor/receptor interactions.

### Example 5

### SrtA-Mediated tethering and Cleavage of tagged GGG-EGF to Pre-Formed PEG Hydrogels Containing LPRTG Substrate

The studies herein demonstrate the simplicity, modularity, and high specificity of sortase-mediated ligation and modification of hydrogels, using tethering of EGF as an example.

Materials and Methods

A. Hydrogel Fabrication and Sortase-Mediated GGG-EGF Tethering

PEG macromers (10 kDa 8-arm PEG-acrylate and 5 kDa 4-arm PEG-thiol) were purchased from JenKem. Amidated cystein-terminated peptides (LPRTG=GCRE-LPRTGGGK-NH₂, LPRTG-fam=GCRE-LPRTGGGK(fluorescein)-NH₂, synKRGD=PHSRNGGGK(GGGERCG-act)-GGRGDSPY and scrambled synKRGD=PHSRNGGGK(GGGERCG-act)-GGRDGSPY) were purchased from Boston Open Labs. The recombinant sortase-tagged GGG-EGF (human epidermal growth factor terminated by a triglycine sequence at the N-terminus) and the triple mutant of the sortase A enzyme (SrtA-3M) (Chen et al., Proceedings of the National Academy of Sciences 108, 11399-11404, 2011) were expressed and purified as previously reported (Krueger et al., Angewandte Chemie International Edition 53, 2662-2666, 2014). Using Michael-type addition as described by Lutolf and Hubbell (Lutolf and Hubbell, Biomacromolecules 4, 713-722, 2003), 5% w/v polymer hydrogels with 1:1 thiols:non-thiols ratio were synthesized by pre-incubating PEG-acrylate macromers with peptides in PBS at pH 6.9 for 20 minutes. PEG-thiol macromers were finally added and 10 μl hydrogels were allowed to form in the inner wells of 96-well angiogenesis plates (0.125 cm²; Ibidi). After gelation, hydrogels were covered with PBS and allowed to swell for 90 minutes at 4° C. with PBS changes every 30 minutes. They were further allowed to swell in intermediate buffer (50 mM HEPES, 150 mM NaCl pH 7.9) for 90 minutes at 4° C. with buffer changes every 30 minutes. Hydrogels were blocked with 70₁1.1/well calcium buffer (50 mM HEPES, 150 mM NaCl, 10 mM CaCl₂, pH 7.9) containing 0.5% purified bovine casein (Sigma-Aldrich) for 30 minutes at room temperature (RT). Tethering solution containing 2 or 20 μM GGG-EGF and 15 μM sortase enzyme in calcium buffer was added on hydrogels (50 μl per well) and reaction was allowed to occur for 1 hour at RT with constant agitation at 30 rpm. Tethering solution without sortase was used to test non-specific binding of GGG-EGF to hydrogels and calcium buffer was used for controls and soluble EGF conditions. Sortase reaction was stopped by addition of 5μl/well EDTA 300 mM. Supernatants were collected and frozen at −80° C. for EGF quantification and hydrogels were extensively washed (3× immediately and every 30 minutes for 3 hours) with 70 μl/well intermediate buffer and soaked overnight at 4° C.

B. Fluorescence Measurements

Hydrogels containing a mix of 96% regular LPRTG and 4% LPRTG-fam peptides were made in 96-well angiogenesis plates as described in Materials and Methods section A above. After gelation and washes in PBS and intermediate buffer, 50 μl of intermediate buffer was added on top of each gel and fluorescence was measured using a microplate reader (SpectraMax M2e from Molecular Devices) and SoftMax Pro 5.4 software. The excitation wavelength was set to 485 nm and the emission wavelength to 530 nm. Plate was scanned without lid using top read mode. Fluorescence was also measured in the same way after overnight swelling following EGF tethering and after washes following EGF cleavage (Materials and Methods section D). Between fluorescence measurements, the plate was protected from light with an aluminum foil.

C. GGG-EGF Detection with Direct ELISA on Hydrogels

Hydrogels were blocked with Odyssey Blocking Buffer (Li-Cor Biosciences) diluted 1:1 with PBS (OBB-PBS) for 1 hour at RT and washed 3× with 0.1% Tween-20 in PBS. Reagents from the DuoSet EGF ELISA kit (R&D Systems, DY236-05) were used for EGF detection. Hydrogels were incubated with biotinylated goat anti-human EGF detection antibody at a concentration of 50 ng/ml in OBB-PBS for 2 hours at RT with constant agitation at 30 rpm. The washing steps were repeated and hydrogels were incubated with streptadivin-HRP diluted 1:40 in OBB-PBS for 20 minutes at RT with constant agitation at 30 rpm and protected from light with an aluminum foil. After washes, hydrogels were incubated with substrate solution consisting of a 1:1 mixture of Color Reagent A (H₂O₂) and Color Reagent B (tetramethylbenzidine) (R&D Systems, DY994) for 20-30 minutes at RT protected from light. The reaction was stopped with 2 N H₂SO₄.20 μl of supernatants were transferred to a clear bottom Nunc MaxiSorp 384-well plate (Thermo Scientific) and optical density at 450 nm was measured using a microplate reader (SpectraMax M2e, Molecular Devices) and SoftMax Pro 5.4 software. Optical density at 540 nm was subtracted to account for optical imperfections of the plate.

D. Sortase-Mediated GGG-EGF Cleavage and Quantification of Tethered EGF with Modified Sandwich ELISA

GGG-EGF-tethered hydrogels were soaked in calcium buffer for 1 hour at 4° C. and incubated for 48 hours at 4° C. with constant agitation with 50 μI/well cleavage solution containing 20 mM GGG peptide (Gly-Gly-Gly, Sigma-Aldrich) and 200 μM sortase in calcium buffer. Supernatants were collected and frozen. Reagents from the DuoSet EGF ELISA kit (R&D Systems, DY236-05) were used for EGF quantification. A clear bottom Nunc MaxiSorp 384-well plate (Thermo Scientific) was coated with mouse anti-human EGF capture antibody diluted at the recommended concentration of 4.0 μg/ml in sterile PBS. The plate was covered with an adhesive strip, spun at 1500 rpm for 3 minutes and incubated overnight at 4° C. with constant agitation. Three washing steps with 100 μl/well 0.1% Tween-20 in PBS were performed at RT using an automatic plate washer (405 Touch Microplate Washer, BioTek). The plate was blocked with 100 μl/well OBB-PBS and incubated for 2 to 6 hours at RT with constant agitation at 30 rpm. Hydrogels supernatants recovered after EGF tethering and EGF cleavage steps were first diluted with calcium buffer containing 1% BSA in 1.5 ml Protein LoBind tubes (Eppendorf) and then serially diluted in 0.5 ml Protein LoBind tubes (Eppendorf). Standard curves were made by serially diluting GGG-EGF in 1% BSA in calcium buffer. After the washing steps, samples were plated and the plate was covered with an adhesive strip, spun at 1500 rpm for 3 min and incubated for 2 hours at room temperature with constant agitation. This process was repeated with the biotinylated goat anti-human EGF detection antibody (R&D Systems, DY236-05) diluted at the recommended working concentration of 50 ng/ml in OBB-PBS. The plate was washed, incubated with streptadivin-HRP (R&D Systems, DY236-05) diluted 1:40 in OBB-PBS and spun at 1500 rpm for 3 minutes protected from light with an aluminum foil. After 20 minutes incubation at room temperature with constant agitation, the plate was washed and incubated with substrate solution for 20-30 minutes at RT protected from light. The reaction was stopped with 2 N H₂SO₄ and optical density at 450 nm was measured as described in Materials and Methods section C.

E. Sources of Endometrial Tissues

Eutopic endometrial biopsies were obtained from 2 premenopausal women in the proliferative phase of the menstrual cycle, who were undergoing surgery for benign gynaecological diseases. Selective criteria included that the patients had regular menstrual cycles (26 to 35 days) and did not undergo hormonal treatment before surgery. A standardized questionnaire was used to document all clinical data. Tissues were collected with the approval of the Partners Human Research Committee and the Massachusetts Institute of Technology Committee on the Use of Humans as Experimental Subjects and with the informed consent of each patient.

F. Primary Endometrial Epithelial Cells Isolation and Purification

Tissues were dissociated and cells purified as described by Osteen and coworkers (Osteen et al. 1989) with some modifications. Biopsy specimens were collected using a pipelle and immediately placed in an ice-cold 1:1 mixture of Dulbecco's Modified Eagle's Medium and Ham's F-12 (Gibco) supplemented with 1% penicillin/streptomycin (DMEM/F12). The tissue was washed twice by centrifugation at 400X g in DMEM/F12 and dissected into small pieces (1-2 mm³). Tissue pieces were incubated for 1 hour at 37° C. in DMEM-F12 supplemented with 0.5% collagenase Type IV (Worthington Biochemical Corporation LS004188), 0.02% DNAase (Sigma-Aldrich DN25) and 2% chicken serum (Sigma-Aldrich C5405) and vortexed every 15 minutes. As a result of this first dissociation, stromal cells are present as single cells while epithelial cells remain aggregated. The cell suspension was then filtered twice through a 70 μm membrane filter (Falcon 352350) in order to separate the stromal cells from the epithelial cell clumps. The latter were collected on the surface of the filters and washed by centrifugation with sterile PBS. Further dissociation of the epithelial aggregates was achieved by incubation with an enzyme mixture supplemented with 0.5% collagenase, 0.1% hyaluronidase (Sigma-Aldrich H3506), 0.1% pronase (Sigma-Aldrich P5147), 0.02% DNAase and 2% chicken serum in PBS for 15-20 minutes at 37° C. in a water bath. The cell preparation was filtered through a 70 μm membrane filter in order to get rid of the remaining stromal cells that were released during this digestion and epithelial cell clumps were collected again and further digested with fresh enzyme mixture for 30-45 minutes at 37° C. This final digestion resulted in small epithelial cell clumps of 50-100 cells that were purified by differential sedimentation at unit gravity as follows. Cells were centrifuged and resuspended in 2 ml DMEM/F12 containing 10% v/v dextran/charcoal treated fetal bovine serum (Atlanta biologicals) (DMEM/F12/FBS). Cells were layered slowly over 10 ml of DMEM/F12/FBS in a sterile 15 ml conical tube and the tube was incubated in an upright position at 37° C., 95% air, 5% CO₂ for 30 minutes. The top 10 ml of sedimentation medium were discarded and the sedimentation step was repeated with the bottom 2 ml. The top 10 ml of sedimentation medium were discarded again and 5 ml of DMEM/F12/FBS were added. Final purification was achieved by selective attachment of any remaining stromal cells to plastic substrate as follows. Cells were seeded in a 75 cm² tissue culture flask and incubated at 37° C., 95% air, 5% CO₂ for 1 hour. Non-attached epithelial cells were collected and cultured in DMEM/F12/FBS at a density of 3×10⁵ cells/ml for 2-3 days. After this plating period, medium was changed every other day for 2-3 days until further use.

G. Endometrial Epithelial Cell Culture on Hydrogels

Before cell seeding, hydrogels were washed with PBS, UV-sterilized for 15 minutes and soaked in DMEM/F12/FBS with or without 20 ng/ml hEGF (Invitrogen PHG0313) for 1 hour. Cultured endometrial epithelial cells (EECs) were trypsinized and seeded on hydrogels and on standard plastic bottoms in 96-well angiogenesis plates (0.125 cm²; Ibidi) at a density of 20,000 cells/cm² (2500 cells/well) in 50 μl/well DMEM/F12/FBS. EECs were incubated at 37° C., 95% air, 5% CO₂. 24 hours after cell seeding, medium was switched to serum-free medium DMEM/F12 with or without 20 ng/ml hEGF and cells were incubated for 16 hours.

H. Human Cryopreserved Hepatocytes Cell Culture on Hydrogels

After a PBS wash and UV-sterilization, hydrogels were soaked in human hepatocyte seeding medium (hHSM; Williams E medium supplemented with 5% FBS, 1 μM hydrocortisone, 1% penicillin/streptomycin (P/S), 4 μg/ml human recombinant insulin, 2 mM GlutaMAX, 15 mM HEPES; CM3000 pack; Life Technologies) with or without 20 ng/ml hEGF (Invitrogen PHG0313) for 1 hour. Cryopreserved hepatocytes (Human Plateable Hepatocytes, Induction Qualified, Hu1663, Life Technologies) were quickly thawed in 37° C. water bath, transferred into 25 ml/vial pre-warmed Cryopreserved Hepatocyte Recovery Medium (CHRM; CM7000; Life Technologies) and centrifuged at RT at 100 g for 8 minutes. Warm hHSM (1 ml) was added to the cell pellet and cells were gently rocked, counted and placed on ice. Hepatocytes were seeded on hydrogels and on collagen I (BioCoat, BD Biosciences) coated wells in 96-well angiogenesis plates (0.125 cm²; Ibidi) at a density of 60,000 cells/cm² (7500 cells/well) in 50₁1.1/well hHSM. Hepatocytes were incubated at 37° C., 95% air, 5% CO₂. 24 hours after cell seeding, medium was switched to serum-free human hepatocyte maintenance medium (hHMIM; Williams E medium supplemented with 0.1 μM hydrocortisone, 0.5% P/S, ITS+(human recombinant insulin (6.25 μg/ml), human transferrin (6.25 μg/ml), selenous acid (6.25 ng/mL), bovine serum albumin (1.25 mg/ml), linoleic acid (5.35 μg/mL)), 2 mM GlutaMAX, 15 mM HEPES; CM4000 pack; Life Technologies) with or without 20 ng/ml hEGF and cells were incubated for 24 hours.

I. DNA Synthesis Assay

Reagents of Click-iT EdU Alexa Fluor 488 kit (Life Technologies) were used for DNA synthesis assay. 40 hours after cell seeding, EECs were incubated with 10 μM of 5-ethynyl-2′-deoxyuridine (EdU) in DMEM/F12 with or without 20 ng/ml hEGF for 24 hours at 37° C., 95% air, 5% CO₂. Similarly, hepatocytes were incubated 48 hours post seeding with 10 μM EdU in hHMIIVI with or without hEGF for 24 hours. Both cell-types were fixed with 3.7% formaldehyde in PBS for 15 minutes at RT. Cells were washed twice with 3% BSA in PBS and permeabilized with 0.5% Triton X-100 in PBS for 20 minutes at RT. Click-iT reaction cocktail was prepared as described by the manufacturer and after the washing steps were repeated, 20 ₁1.1 per well were added. Cells were incubated for 30 minutes at RT protected from light with an aluminum foil. After cells were washed once with 3% BSA in PBS and once with PBS, they were incubated with Hoechst 33342 diluted 1:2000 in PBS for 30 minutes at RT protected from light. Cells were finally washed twice with PBS and imaged using a Leica DMI 6000 microscope and Oasis Surveyor software. Images were processed and cell nuclei were counted using ImageJ64 software. The percentage of cells synthesizing DNA was computed as the ratio of cells positively stained with Alexa Fluor 488 divided by the total number of cells given by Hoechst counter-staining.

Results

SrtA-mediated tethering of epidermal growth factor (EGF) to pre-formed PEG hydrogels enhances DNA synthesis of primary epithelial cells. Characterization herein shows that the amount of tethered EGF increases with both the concentration of LPRTG in hydrogels and the concentration of GGG-EGF, in agreement with enzymatic kinetics associated with a ping-pong mechanism. DNA synthesis assays with human primary hepatocytes and endometrial epithelial cells validated the biological activity of tethered EGF, which considerably stimulated DNA synthesis for both cell types compared to unmodified hydrogels.

Hydrogels were formed by copolymerizing acrylate-terminated multi-arm PEG with a Cys-terminated LPRTG peptide through Michael-type addition as reported by Lutolf and Hubbell (Lutolf and Hubbell, Biomacromolecules 4, 713-722, 2003). Subsequently, N-terminal Gly₃-tagged epidermal growth factor (GGG-EGF) was tethered mainly close to the surface of the hydrogels through Sortase A (triple mutant “SrtA 3M”)-mediated ligation (SML). EGF was chosen as a model growth factor as it has been extensively studied since its discovery in 1986. Cell functions influenced by EGF receptor (EGFR) signaling include mitogenesis, apoptosis, migration, protein secretion, and differentiation. Moreover, an attractive feature to explore with tethered EGF compared to the soluble form is the supposed sustained signaling caused by the fact that the bound EGFR cannot be internalized (Ito, Soft Matter 4, 46, 2008). EGFR signaling is also known to interact with several mechanisms, including integrins signaling, in order to modulate cell adhesion and migration.

As described herein, to promote cell adhesion on hydrogels, PHSRN-RGD peptide (synKRGD) was used, which is an improved variant of the well-known RGD peptide. Indeed, cell adhesion through ⊕₅β₁ integrin is enhanced by synergistic sites in the cell-adhesive domain of fibronectin and the short peptide sequence PHSRN was found to be the minimal sequence to promote synergistic activity. Motivated by the abundant EGFR expression of hepatocytes (over 250,000 EGFR per cell in primary rat hepatocytes) and by several studies showing the phenotypic effects of soluble and tethered EGF on this cell-type (Kuhl and Griffith-Cima Nature medicine, 2(9), 1022-1027, 1996; Mehta, et al., Biomaterials 31, 4657-46712010; Williams et al., Tissue Engineering Part A 17, 1055-1068, 2011), human primary hepatocytes were chosen to validate the biological activity of the sortase-tethered EGF. Although poorly studied until now, endometrial epithelial cells (EECs) have recently raised great interest owing to their implication in cancer, endometriosis, and fertility. Cultured in presence of soluble EGF, human EECs as well as endometrial cancer cell lines were shown to possess EGFRs, thus also rendering them an attractive cell-type to seed on EGF-modified hydrogels.

A. Quantification of SrtA-Mediated GGG-EGF Tethering with Sandwich ELISA

SrtA-mediated ligation is highly specific. Here, the ability of SrtA-3M to recognize the LPRTG substrate incorporated in PEG hydrogels and to specifically tether the GGG-EGF substrate was examined. PEG hydrogels containing 0, 20, 50, 100 or 250 μM LPRTG peptide were cross-linked through Michael-type addition as schematized in FIG. 4A. Tethering solution containing 15 μM sortase and 2 or 20 μM GGG-EGF was then added on top of pre-formed hydrogels for SML, which is illustrated in FIG. 4B. The amount of GGG-EGF present in initial tethering solution and in hydrogels supernatants post tethering was quantified with sandwich ELISA. FIGS. 5A and 5B display the amount of GGG-EGF as a function of LPRTG concentration in hydrogels for tethering with 2 and 20 μM GGG-EGF respectively. Interestingly, for both GGG-EGF concentrations, the amount of GGG-EGF in supernatants post-reaction decreases with LPRTG concentration in hydrogels. However, for tethering at 2μM GGG-EGF, the decrease was fast at lower LPRTG concentration and slowed down at higher substrate concentration with a hyperbolic trend reminiscent of Michaelis-Menten kinetics. The decrease seemed more continuous with 20 μM GGG-EGF in solution. GGG-EGF may thus represent the limiting factor of the reaction when tethered at 2 μM. In both cases, the dependence on LPRTG concentration suggests that at least part of the GGG-EGF substrate is tethered to LPRTG substrate through sortase reaction. Another part of GGG-EGF likely diffuses through the hydrogel without being bound and, finally, a third part may non-specifically stick to the hydrogel. The hypothesis of non-specific binding is in part corroborated by the fact that the amount of GGG-EGF after tethering with 20 μM GGG-EGF and 0μM LPRTG in hydrogel is significantly lower than the initial amount of EGF in tethering solution. Remarkably, for each LPRTG concentration the amount of GGG-EGF in supernatants post tethering is about ten times higher for the 20 μM tethering compared to the 2 μM one. This suggests an enzymatic kinetics that is linearly dependent on the concentration of the GGG-EGF substrate. All these findings match a ping-pong mechanism, described by equation 1 (Huang et al., Biochemistry, 42(38), 11307-11315, 2003).

This enzymatic kinetic is typical of transpeptidases reactions where the enzyme first binds to a primary substrate and is converted to an intermediate enzyme, which can then bind and process a secondary substrate. In the case of sortase, the enzyme first recognizes an LPXTG sequence and cleaves the amide bond between the threonine and the glycine yielding a thioacyl intermediate. This intermediate is then resolved by the N-terminus of an oligogylcine nucleophile. In absence of a nucleophile, water resolves the intermediate and a hydrolysis product is formed.

B. Fluorescence Measurement of Sortase-Mediated GGG-EGF Tethering

To determine the amount of LPRTG molecules effectively processed by sortase, 4% of the LPRTG peptide in hydrogel was replaced by LPRTG-fluorescein (LPRTG-fam). Fluorescein was meant to act as an indicator of the proper functioning of the site-specific enzymatic reaction. Indeed, as the sortase enzyme cleaves the peptide bond between the threonine and the glycine of the LPRTG motif, fluorescein is released, thus resulting in fluorescence decrease. A linear standard curve (R²=0.9994) was established by measuring the fluorescence of hydrogels containing 0, 20, 50, 100 or 250 μM of total LPRTG peptide (FIG. 6A). It allowed conversion of fluorescent units into supposed amount of LPRTG in pmol. Fluorescence was measured before and after GGG-EGF SML and controls allowed estimating photobleaching and potential non-specific release of the fluorescein. FIG. 6B displays the amount of reacted LPRTG as a function of initial LPRTG concentration in hydrogels. This metric was obtained by subtracting final hydrogels fluorescence from initial fluorescence and by converting the result in pmol. Estimated amount of photobleached LPRTG obtained from the controls was then subtracted. Interestingly, photobleaching unexpectedly increased from 2 to 5% of the initial fluorescence between 20 and 250 μM (data not shown). Corrected data show that the amount of reacted LPRTG increases with LPRTG concentration in a hyperbolic manner for both 2 and 20 μM GGG-EGF concentrations. Again, the increase is much slower at 2μM GGG-EGF (FIG. 6B). This suggests that the evolved triple mutant enzyme (SrtA-3M) is capable of recognizing the LPRTG motif incorporated in the hydrogels and of cleaving the peptide bond between the threonine and the glycine, thereby releasing the fluorescein. Moreover these data seem to support again ping-pong kinetics.

C. Direct ELISA on Hydrogels

To verify site-specific sortase-mediated EGF tethering, the presence of EGF on the hydrogels was detected with direct ELISA after tethering of 2 or 20 μM GGG-EGF. Non-specific binding was tested after incubation of the hydrogels with 2 or 20 μM GGG-EGF in absence of sortase. Hydrogels were extensively washed and successively incubated with biotinylated detection antibody, streptadivin-HRP and substrate solution. Optical density of hydrogels supernatants was measured using a plate reader. While the amount of EGF bound at 2 μM without sortase yielded a measurement that was constant and only slightly higher than background, non-specific binding at 20 μM GGG-EGF was higher and appeared to fluctuate (FIG. 7A). Non-specific binding, which seems to be enhanced by a higher concentration of GGG-EGF in tethering solution, may be due to a combination of non-covalent interactions like ionic, hydrophobic or Van der Waals forces. Nevertheless, the amount of non-specifically bound GGG-EGF remained considerably lower than the amount of GGG-EGF bound with sortase. Surpisingly, the presence of sortase seems to increase non-specific binding in the particular case where the LPRTG substrate is absent, suggesting that sortase can bind to the hydrogel and affinity capture GGG-EGF. In order to compare sortase-mediated tethering at 2 and 20 μM GGG-EGF, measurements performed in absence of sortase were subtracted from measurements performed in presence of the enzyme (FIG. 7B). Remarkably, a steady and identical increase in the amount of detected GGG-EGF was observed between 0 and 50 μM LPRTG in hydrogel for both GGG-EGF concentrations (FIG. 7B). Starting just before 50 μM LPRTG, the two curves continued to increase more slowly and separately, with the 2 μM GGG-EGF curve increasing less than the 20 μM GGG-EGF curve. The fact that the amount of bound GGG-EGF is similar for both GGG-EGF concentrations until around 50 μM LPRTG may indicate that LPRTG is likely a limiting factor below 50 μM in hydrogel, while beyond this concentration GGG-EGF becomes a limiting factor.

D. Sortase-Mediated Hydrogels Cleavage and Mass Balance

A noteworthy aspect of sortase-mediated tethering is that the product formed contains a LPRTGGG sequence that becomes itself a potential substrate. This feature was beneficially used to quantify the amount of tethered GGG-EGF after cleaving it using sortase (FIG. 8). In order to assess completeness of the cleavage, fluorescein was used again as an indicator. After 2 or 20 μM GGG-EGF tethering, hydrogels containing 0, 20, 50, 100 or 250 μM of total LPRTG at a ratio of 4% LPRTG-fam and 96% regular LPRTG were incubated with 20 mM GGG and 200 μM sortase for 48 hours at 4° C. Fluorescence was measured as previously described. After hydrogels cleavage, i.e. cleavage of previously tethered GGG-EGF plus LPRTG and LPRTG-fam, the percentage of retained fluorescence dropped to a similar level of 9 to 12% of initial fluorescence for all LPRTG concentrations and for both GGG-EGF concentrations (FIG. 9). Differences seem to be due to photobleaching, which slightly increased with LPRTG concentration from 5 to 8% of initial fluorescence (FIG. 9). Residual fluorescence may be caused by inaccessibility of certain LPRTG-fam molecules or possible attachment of fluorescein to the plastic of the plate. Reaching the same low level of fluorescence for all the conditions ensures complete cleavage of the hydrogels and thus full release of previously tethered GGG-EGF in solution.

Hydrogels supernatants were collected after washes following tethering and after hydrogel cleavage; GGG-EGF was quantified as previously described with a modified sandwich ELISA. FIGS. 10A and 10B present the amount of released GGG-EGF as a function of LPRTG concentration for tethering at 2 and 20 μM GGG-EGF respectively. For both concentrations, the amount of GGG-EGF released during washes after tethering is low, constant, and proportional to the initial concentration of GGG-EGF used. Cleaved GGG-EGF increased with LPRTG concentration in hydrogels, thus confirming previous experiments. Although the amount of cleaved GGG-EGF should match the amount of reacted LPRTG peptides yielded by fluorescence measurements, it is possible that the latter may be overestimated due to photobleaching and hydrolysis. On the contrary, the amount of cleaved GGG-EGF is possibly underestimated due to probable loss of protein during freezing and dilutions of the supernatants. Notably, when the amount of GGG-EGF remaining in solution after tethering was combined with the amount of GGG-EGF released by washes and the amount of cleaved GGG-EGF, this sum completed the mass balance of GGG-EGF (FIGS. 11A and 11B). Differences observed when comparing to the initial amount of GGG-EGF may be due to non-specifically bound GGG-EGF. This seems to be especially the case for hydrogels lacking LPRTG substrate for tethering at 2μM GGG-EGF, which, in the absence of peptide, offer a wider surface for non-specific binding (FIG. 11A). Non-specific binding seems to be higher and more uniform relatively to the LPRTG concentration for tethering at 20 μM GGG-EGF (FIG. 11B). Based on these experiment, experiments will be performed on hydrogels containing 250 μM LPRTG, in order to maximize tethered EGF surface density, with 2μM GGG-EGF in tethering solution in order to minimize non-specific binding.

E. Cell Attachment and DNA Synthesis Assay with Cryopreserved Human Hepatocytes and Primary Endometrial Epithelial Cells

To validate the system and the biological activity of the sortase-tethered GGG-EGF, human hepatocytes and primary endometrial epithelial cells (EECs) were cultured on hydrogels containing 0 or 500 μM synKRGD adhesion peptide. The concentration of 500 μM synKRGD was chosen as it was found to be an optimal intermediate concentration, which was sufficient to induce cell attachment and which did not mask the effect of EGF (data not shown). Since primary hepatocytes are a well-known EGF-responsive cell type, they serve as a good means of comparison to investigate EGF response of less studied but nonetheless interesting EECs. Hepatocytes and EECs were also cultured on collagen I coated bottom and on standard plastic bottom respectively. Cells were either unexposed to EGF or were presented with GGG-EGF tethered at 2μM on 250 μM LPRTG hydrogels or with soluble human EGF (hEGF, Invitrogen) at 20 ng/ml. Incubation with EdU was performed 48 hours after seeding for 24 hours for hepatocytes and 40 hours after seeding for 24 hours for EECs. In absence of EGF, while only a small number of cells attached on hydrogels without adhesion peptide, cell attachment on 500 μM synKRGD hydrogels was comparable to positive controls on collagen I or standard plastic bottom (FIGS. 12A and 13A). Interestingly, tethered EGF had no effect on hepatocyte attachment, contrary to soluble hEGF, which slightly enhanced attachment on 500 μM synKRGD hydrogels compared to unexposed hydrogels, and which yielded a 1.8 fold increase on collagen I coated bottoms. On the other hand, tethered EGF was as effective as the soluble form in promoting EECs adhesion both on 0 and 500 μM synKRGD hydrogels. As expected, soluble EGF also substantially enhanced EECs attachment on standard plastic substrates.

As to hepatocytes DNA synthesis, soluble EGF considerably and similarly increased basal DNA synthesis from 9 to 21% on 500 μM synKRGD hydrogels and from 10 to 22% on standard plastic. However, tethered EGF seems to stimulate DNA synthesis significantly more than soluble EGF, by essentially triplicating basal DNA synthesis from 9 to 27% on 500 μM synKRGD hydrogels. Remarkably, soluble EGF had the same effect on EECs as on hepatocytes, by doubling basal DNA synthesis from 6 to 12 and 15% on 500 μM synKRGD hydrogels and plastic respectively. While tethered EGF significantly improved DNA synthesis from 6 to 14% compared to unexposed hydrogels, this performance was only slightly better than the one of soluble EGF.

As the results herein demonstrate, the SrtA-mediated approach is not only simple and relatively inexpensive, but also displays high specificity and modularity. The reversibility of the sortase reaction, which is usually perceived as a defect, was used beneficially to cleave previously bound EGF and perform absolute quantification experiments with a sandwich ELISA assay. Supported by fluorescence measurement, results showed that the amount of grafted growth factor was readily controlled by the amount of LPRTG substrate incorporated in hydrogels and the amount of GGG-EGF present in solution, suggesting ping-pong enzymatic kinetics. While the sortase enzyme was capable of recognizing and processing LPRTG peptides that were incorporated in the hydrogels through Michael-type addition, this method represents a useful tool for specific modification of any type of hydrogel containing the appropriate substrates. Helped by the well-known anti-fouling properties of the PEG polymer as well as enzymatic specificity, reduction of GGG-EGF concentration in initial tethering solution allowed minimizing non-specific binding. Finally, biological activity of the tethered EGF was confirmed by the phenotypic response of human primary epithelial cells. The well-studied hepatocyte cell-type validated the system while preliminary experiments with EECs constituted a first example of EECs culture on hydrogels in presence of a tethered growth factor. Overall, SrtA-mediated ligation of bioactive molecules to hydrogels promises to contribute to the development of improved culture systems for in vitro models, to closely recapitulate the ECM.

### REFERENCES

1. Griffith, L. G. & Swartz, M. A. Capturing complex 3D tissue physiology in vitro. Nat. Rev. Mol. Cell Biol. 7,211-224 (2006).

2. Lutolf, M. P. & Hubbell, J. A. Synthetic biomaterials as instructive extracellular microenvironments for morphogenesis in tissue engineering. Nat Biotechnol 23, 47-55 (2005).

3. Burdick, J. A. & Murphy, W. L. Moving from static to dynamic complexity in hydrogel design. Nature Communications 3,1269 (2012).

4. Paszek, M. J. et al. Tensional homeostasis and the malignant phenotype. Cancer Cell 8, 241-254 (2005).

5. Osteen, K. G. et al. Stromal-epithelial interaction mediates steroidal regulation of metalloproteinase expression in human endometrium. Proc. Natl. Acad. Sci. U.S.A. 91, 10129-10133 (1994).

6. Rothberg, J. M., Sameni, M., Moin, K. & Sloane, B. F. Live-cell imaging of tumor proteolysis: Impact of cellular and non-cellular microenvironment. Biochimica et Biophysica Acta (BBA)−Proteins and Proteomics 1824, 123-132 (2012).

7. Camp, J. T. et al. Interactions with Fibroblasts Are Distinct in Basal-Like and Luminal Breast Cancers. Molecular Cancer Research 9, 3-13 (2011).

8. Chen, I., Dorr, B. M. & Liu, D. R. A general strategy for the evolution of bond-forming enzymes using yeast display. Proceedings of the National Academy of Sciences 108, 11399-11404 (2011).

9. Popp, M. W.-L. & Ploegh, H. L. Making and Breaking Peptide Bonds: Protein Engineering Using Sortase. Angew. Chem. Int. Ed. 50, 5024-5032 (2011).

10. Chan, L. et al. Covalent Attachment of Proteins to Solid Supports and Surfaces via Sortase-Mediated Ligation. PLoS ONE 2, e1164 (2007).

11. Liu, F., Luo, E. Y., Flora, D. B. & Mezo, A. R. Irreversible Sortase A-Mediated Ligation Driven by Diketopiperazine Formation. J. Org. Chem. 79, 487-492 (2014).

12. Miller, M. A. et al. ADAM-10 and -17 regulate endometriotic cell migration via concerted ligand and receptor shedding feedback on kinase signaling. Proceedings of the National Academy of Sciences 110, E2074-E2083 (2013).

13. Cosgrove, B. D. et al. An inducible autocrine cascade regulates rat hepatocyte proliferation and apoptosis responses to tumor necrosis factor-alpha. Hepatology 48, 276-288 (2008).

14. Sperinde, J. J. & Griffith, L. G. Control and Prediction of Gelation Kinetics in Enzymatically Cross-Linked Poly(ethylene glycol) Hydrogels. Macromolecules 33, 5476-5480 (2000).

15. Ehrbar, M. et al. Biomolecular Hydrogels Formed and Degraded via Site-Specific Enzymatic Reactions. Biomacromolecules 8, 3000-3007 (2007).

16. Kobel, S. & Lutolf, M. P. Biomaterials meet microfluidics: building the next generation of artificial niches. Curr Opin Biotechnol 22, 690-697 (2011).

17. Lutolf, M. P., Gilbert, P. M. & Blau, H. M. Designing materials to direct stem-cell fate. Nature 462, 433-441 (2009).

18. Ehrbar, M. et al. Enzymatic formation of modular cell-instructive fibrin analogs for tissue engineering. Biomaterials 28, 3856-3866 (2007).

19. Teixeira, L. S. M., Feijen, J., van Blitterswijk, C. A., Dijkstra, P. J. & Karperien, M. Biomaterials. Biomaterials 33, 1281-1290 (2012).

20. Zhu, J. Biomaterials. Biomaterials 31, 4639-4656 (2010).

21. Lei, Y. & Schaffer, D. V. A fully defined and scalable 3D culture system for human pluripotent stem cell expansion and differentiation. Proceedings of the National Academy of Sciences 110, E5039-48 (2013).

22. Zhang, J., Skardal, A. & Prestwich, G. D. Engineered extracellular matrices with cleavable crosslinkers for cell expansion and easy cell recovery. Biomaterials 29, 4521-4531 (2008).

23. Billiet, T., Vandenhaute, M., Schelfhout, J., Van Vlierberghe, S. & Dubruel, P. A review of trends and limitations in hydrogel-rapid prototyping for tissue engineering. Biomaterials 33, 6020-6041 (2012).

24. DeForest, C. A. & Anseth, K. S. Photoreversible Patterning of Biomolecules within Click-Based Hydrogels. Angew. Chem. Int. Ed. 51, 1816-1819 (2011).

25. Tibbitt, M. W., Kloxin, A. M. & Anseth, K. S. Modeling controlled photodegradation in optically thick hydrogels. J. Polym. Sci. A Polym. Chem. 51, 1899-1911 (2013).

26. Griffith, L. G. & Lopina, S. Microdistribution of substratum-bound ligands affects cell function: hepatocyte spreading on PEO-tethered galactose. Biomaterials 19, 979-986 (1998).

27. Maheshwari, G., Brown, G., Lauffenburger, D. A., Wells, A. & Griffith, L. G. Cell adhesion and motility depend on nanoscale RGD clustering. Journal of Cell Science 113 (Pt 10), 1677-1686 (2000).

28. Koo, L. Y., Irvine, D. J., Mayes, A. M., Lauffenburger, D. A. & Griffith, L. G. Co-regulation of cell adhesion by nanoscale RGD organization and mechanical stimulus. Journal of Cell Science 115, 1423-1433 (2002).

29. Williams, C. M. et al. Autocrine-controlled formation and function of tissue-like aggregates by primary hepatocytes in micropatterned hydrogel arrays. Tissue Eng Part A 17, 1055-1068 (2011).

30. Mehta, G. et al. Synergistic effects of tethered growth factors and adhesion ligands on DNA synthesis and function of primary hepatocytes cultured on soft synthetic hydrogels. Biomaterials 31, 4657-4671 (2010).

31. Marcantonio, N. A. et al. The influence of tethered epidermal growth factor on connective tissue progenitor colony formation. Biomaterials 30, 4629-4638 (2009).

32. Kuhlman, W., Taniguchi, I., Griffith, L. G. & Mayes, A. M. Interplay between PEO tether length and ligand spacing governs cell spreading on RGD-modified PMMA-g-PEO comb copolymers. Biomacromolecules 8, 3206-3213 (2007).

33. Stains, C. I. et al. Interrogating Signaling Nodes Involved in Cellular Transformations Using Kinase Activity Probes. Chemistry & Biology 19, 210-217 (2012).

34. Cosgrove, B. D. et al. Cytokine-associated drug toxicity in human hepatocytes is associated with signaling network dysregulation. Mol Biosyst 6, 1195-1206 (2010).

35. Krueger, A. T., Kroll, C., Sanchez, E., Griffith, L. G. & Imperiali, B. Tailoring Chimeric Ligands for Studying and Biasing ErbB Receptor Family Interactions. Angew. Chem. Int. Ed. 53, 2662-2666 (2014).

36. Fairbanks, B. D. et al. A Versatile Synthetic Extracellular Matrix Mimic via Thiol-Norbornene Photopolymerization. Adv. Mater. 21, 5005-5010 (2009).

37. Zustiak, S. P., Durbal, R. & Leach, J. B. Acta Biomaterialia. Acta Biomaterialia 6, 3404-3414 (2010).

38. Fan, V. H. et al. Tethered Epidermal Growth Factor Provides a Survival Advantage to Mesenchymal Stem Cells. Stem Cells 25, 1241-1251 (2007).

39. Rodrigues, M., Griffith, L. G. & Wells, A. Growth factor regulation of proliferation and survival of multipotential stromal cells. Stem Cell Res Ther 1, 32 (2010).

40. Kreiner, M. et al. Self-assembling multimeric integrin 5 1 ligands for cell attachment and spreading. Protein Engineering Design and Selection 21, 553-560 (2008).

41. Miller, M. A. et al. Proteolytic Activity Matrix Analysis (PrAMA) for simultaneous determination of multiple protease activities. Integr Biol (Camb) 3, 422-438 (2011).

42. Singh, B. & Coffey, R. J. Trafficking of Epidermal Growth Factor Receptor Ligands in Polarized Epithelial Cells. Annu. Rev. Physiol. 76, 275-300 (2014).

43. Kuhl, P. R. & Griffith-Cima, L. G. Tethered epidermal growth factor as a paradigm for growth factor-induced stimulation from the solid phase. Nat. Med. 2, 1022-1027 (1996).

44. SEMINO, C., KAMM, R. & LAUFFENBURGER, D. Autocrine EGF receptor activation mediates endothelial cell migration and vascular morphogenesis induced by VEGF under interstitial flow. Experimental Cell Research (2005). doi:10.1016/j.yexcr.2005.10.029

45. Osteen, K. G., Hill, G. A., Hargrove, J. T. & Gorstein, F. Development of a method to isolate and culture highly purified populations of stromal and epithelial cells from human endometrial biopsy specimens. Fertility and Sterility 52, 965-972 (1989).

46. Lutolf, M. P. & Hubbell, J. A. Synthesis and Physicochemical Characterization of End-Linked Poly(ethylene glycol)- co-peptide Hydrogels Formed by Michael-Type Addition. Biomacromolecules 4, 713-722 (2003).

47. Peyton, S. R. et al. Marrow-derived stem cell motility in 3D synthetic scaffold is governed by geometry along with adhesivity and stiffness. Biotechnol. Bioeng. 108, 1181-1193 (2011).

48. Oelker, A. M., Morey, S. M., Griffith, L. G. & Hammond, P. T. Helix versus coil polypeptide macromers: gel networks with decoupled stiffness and permeability. Soft Matter 8, 10887 (2012).

### Incorporation By Reference

The relevant teachings of all patents, published applications, and references cited herein are incorporated by reference in their entirety.

### Equivalents

While this invention has been particularly shown and described with references to examples of embodiments thereof, it will be understood by those skilled in the art that various changes in form and details may be made therein without departing from the scope of the invention encompassed by the appended claims.

1. A hydrogel comprising one or more scaffold macromers crosslinked to a mixture of peptides, wherein all or a portion of the peptides in the mixture comprise a recognition motif cleavable by a transpeptidase.

2. The hydrogel of claim 1, wherein the transpeptidase is a sortase or a sortase variant.

3. The hydrogel of claim 1, wherein the recognition sequence comprises a motif selected from the group consisting of: LPXSG, LPXTG, and LAXTG.

4. The hydrogel of any of claim 1, wherein 0.001% to 80% of the peptides in the mixture comprise a recognition motif cleavable by a first transpeptidase.

5. The hydrogel of claim 4, wherein each peptide that comprises a first recognition sequence cleavable by the first transpeptidase does not comprise a second recognition motif cleavable by a second transpeptidase.

6. The hydrogel of any of claim 1, wherein the peptide further comprises a sequence cleavable by a protease.

7. The hydrogel of claim 6, wherein the protease is an endopeptidase or a metalloprotease.

8. The hydrogel of claim 1, wherein the peptide comprises the amino acid sequence GCRDLPRTGGPQGIWGQDRCG.

9. The hydrogel of claim 1, wherein a portion of the peptides in the mixture is crosslinked to a macromer at its N-terminus, and is free at its C-terminus.

10. The hydrogel of claim 1, wherein the hydrogel encapsulates a cell, a tissue, or an organ.

11. The hydrogel of claim 1, wherein the scaffold macromer is selected from any one or more of polyethyleneglycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin.

12. The hydrogel of claim 11, wherein the PEG is a linear or a branched PEG.

13. The hydrogel of claim 11, wherein the water-soluble polypeptide is a branched polypeptide.

14. A method of forming a hydrogel dissolvable by a transpeptidase, said method comprising:
combining
1) a mixture of peptides, wherein all or a portion of the peptides in the mixture comprise a recognition motif cleavable by a transpeptidase, each peptide having a first crosslinking moiety;
2) one or more scaffold macromers having a second crosslinking moiety; and
3) a suitable crosslinking agent
under suitable conditions that promote crosslinking of the first and second crosslinking moieties, thereby forming a hydrogel dissolvable by a transpeptidase.

15. The method of claim 14, wherein the transpeptidase is a sortase or a sortase variant.

16. The method of claim 14, wherein the recognition motif comprises a sequence selected from the group consisting of: LPXSG, LPXTG, and LAXTG.

17. The method of claim 14, wherein 0.001% to 80% of the peptides in the mixture comprise a recognition motif cleavable by a first transpeptidase.

18. The method of claim 17, wherein a peptide that comprises a first recognition motif cleavable by a first transpeptidase does not comprise a second recognition motif cleavable by a second transpeptidase.

19. The method of claim 14, wherein the peptide further comprises a sequence cleavable by a protease.

20. The method of claim 19, wherein the protease is an endoprotease or a metalloprotease.

21. The method of claim 14, wherein the peptide comprises the amino acid sequence GCRDLPRTGGPQGIWGQDRCG.

22. The method of claim 14, wherein the mixture of peptides further comprises a terminal peptide having a recognition motif cleavable by a transpeptidase, said terminal peptide having a crosslinking moiety on one end.

23. The method of claim 14, further comprising combining a cell, a tissue, or an organ.

24. The method of claim 14, wherein the scaffold macromer is selected from any one or more of polyethylene glycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin.

25. The method of claim 24, wherein the PEG is a linear or branched PEG.

26. The method of claim 24, wherein the water-soluble polypeptide is a branched polypeptide.

27. A method of dissolving the hydrogel of claim 1, said method comprising treating the hydrogel with a first transpeptidase and a peptide comprising an acceptor substrate sequence of the first transpeptidase under conditions that promote dissolution of the hydrogel, thereby dissolving the hydrogel.

28. The method of claim 27, wherein the acceptor substrate sequence comprises NH₂-(G)ₙ, wherein n is equal to or greater than 1.

29. A method of dissolving a hydrogel, said method comprising:
treating a hydrogel comprising a transpeptidase recognition motif with a transpeptidase and a peptide comprising an acceptor substrate sequence under conditions that promote dissolution of the hydrogel,
thereby dissolving the hydrogel.

30. The method of claim 29, wherein the transpeptidase is a sortase or a sortase variant.

31. The method of claim 29, wherein the transpeptidase recognition motif comprises a sequence selected from the group consisting of: LPXSG, LPXTG, and LAXTG.

32. The method of claim 29, wherein the acceptor substrate sequence comprises NH₂-(G)ₙ, wherein n is equal to or greater than 1.

33. The method of claim 29, wherein the acceptor substrate sequence comprises NH₂-triglycine (GGG).

34. The method of claim 29, wherein the hydrogel encapsulates a cell, a tissue, or an organ.

35. The method of claim 29, wherein the hydrogel is pretreated with sortase prior to treatment with the peptide.

36. The method of claim 29, wherein the hydrogel is sufficiently dissolved to release the cell, tissue, or organ.

37. A method of forming a hydrogel comprising a pendant transpeptidase recognition motif, said method comprising:
combining one or more scaffold macromers having a first crosslinking moiety, a peptide comprising a transpeptidase recognition motif having a second crosslinking moiety at its N-terminal end, and a suitable crosslinking agent under conditions that promote crosslinking of the first and second crosslinking moieties,
thereby forming a hydrogel comprising a pendant transpeptidase recognition motif.

38. The method of claim 37, wherein the transpeptidase is a sortase.

39. The method of claim 38, wherein the transpeptidase recognition motif comprises a sequence selected from the group consisting of: LPXSG, LPXTG, and LAXTG.

40. The method of claim 39, wherein the transpeptidase recognition motif comprises LPRTG.

41. The method of claim 37 further comprising treating the hydrogel with a biomolecule having an acceptor substrate sequence that comprises NH₂-(G)ₙ, where n is equal to or greater than 1.

42. The method of claim 41, wherein the acceptor substrate sequence comprises NH₂-triglycine (GGG).

43. The method of claim 41, wherein the biomolecule is a growth factor or an adhesion factor.

44. The method of claim 37, wherein the scaffold macromer is selected from any one or more of polyethyleneglycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin.

45. A method of forming a functionalized hydrogel, said method comprising:
combining
a first scaffold macromer having a terminal transpeptidase recognition motif;
a second scaffold macromer having a terminal transpeptidase acceptor substrate sequence;
one or more biomolecules having a terminal transpeptidase recognition motif or an acceptor substrate sequence; and
a transpeptidase under conditions that promote transpeptidase ligation of the transpeptidase recognition motif with the acceptor substrate sequence,
thereby forming a functionalized hydrogel.

46. The method of claim 45, wherein the biomolecule is a growth factor or an adhesion factor.

47. The method of claim 45, wherein the biomolecule is an epidermal growth factor (EGF) or Neuregulin-1 (NRG).

48. The method of claim 45, wherein the first or second scaffold macromer is selected from any one or more of polyethyleneglycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin.

49. The method of claim 27, wherein the transpeptidase is a sortase.

50. The method of claim 27, wherein the transpeptidase is Sortase A.

51. The method of claim 45, wherein the transpeptidase recognition motif comprises a sequence selected from the group consisting of: LPXSG, LPXTG, and LAXTG.

52. The method of claim 51, wherein the transpeptidase recognition motif comprises LPRTG.

53. The method of claim 45, wherein the transpeptidase acceptor substrate sequence comprises NH₂-(G)ₙ, where n is equal to or greater than 1.

54. The method of claim 45, wherein the transpeptidase acceptor substrate sequence comprises NH₂-triglycine (GGG).

55. A kit for hydrogel formation comprising:
an isolated transpeptidase enzyme; and
a plurality of scaffold macromers, wherein said plurality comprises at least a first macromer having a terminal transpeptidase recognition motif, and at least a second macromer having a terminal transpeptidase acceptor substrate sequence.

56. The kit of claim 55, further comprising a suitable buffer.

57. The kit of claim 55 wherein the scaffold macromer is selected from any one or more of polyethyleneglycol (PEG), a dextran, hyaluronic acid, nipaam, alginate, polyacrylic acid, polyhydroxymethacrylate, elastin polypeptide, silk polypeptide, water-soluble polypeptide, chitosan, agarose, heparin sulfate, or heparin.

58. The kit of claim 55, wherein the transpeptidase is a sortase.

59. The kit of claim 58, wherein the sortase is a modified Sortase A.

60. The kit of claim 55, wherein the transpeptidase acceptor substrate sequence comprises NH₂-(G)ₙ, wherein n is equal to or greater than 1.

61. The kit of claim 55, wherein the transpeptidase acceptor substrate sequence comprises NH₂-triglycine (GGG).

62. The kit of claim 55, wherein the at least first macromer and the at least second macromer are provided in separate containers.


SEQUENCE LISTING (31 sequences)

SEQ ID NO: 1 - protein, 21 aa, Artificial Sequence
  peptide
GCRDLPRTGGPQGIWGQDRCG

SEQ ID NO: 2 - protein, 5 aa, Artificial Sequence
  peptide
LPRTG

SEQ ID NO: 3 - protein, 5 aa, Artificial Sequence
  peptide
  VARIANT 3: Xaa = Any Amino Acid
  VARIANT 3: Xaa = Any Amino Acid
LPXTG

SEQ ID NO: 4 - protein, 7 aa, Artificial Sequence
  peptide
  VARIANT 3: Xaa = Any Amino Acid
  VARIANT 3: Xaa = Any Amino Acid
LPXTGGG

SEQ ID NO: 5 - protein, 5 aa, Artificial Sequence
  peptide
  VARIANT 3: Xaa = Any Amino Acid
  VARIANT 3: Xaa = Any Amino Acid
LPXSG

SEQ ID NO: 6 - protein, 5 aa, Artificial Sequence
  peptide
  VARIANT 3: Xaa = Any Amino Acid
  VARIANT 3: Xaa = Any Amino Acid
LAXTG

SEQ ID NO: 7 - protein, 7 aa, Artificial Sequence
  peptide
LPRTGGG

SEQ ID NO: 8 - protein, 12 aa, Artificial Sequence
  peptide
GCRELPRTGGGK

SEQ ID NO: 9 - protein, 6 aa, Artificial Sequence
  peptide
CLPRTG

SEQ ID NO: 10 - protein, 4 aa, Artificial Sequence
  peptide
GGGC

SEQ ID NO: 11 - protein, 9 aa, Artificial Sequence
  peptide
GCRDRGDSP

SEQ ID NO: 12 - protein, 4 aa, Artificial Sequence
  peptide
LPTG

SEQ ID NO: 13 - protein, 8 aa, Artificial Sequence
  peptide
LPRTGGGK

SEQ ID NO: 14 - protein, 11 aa, Artificial Sequence
  peptide
GGGTTSSERCG

SEQ ID NO: 15 - protein, 14 aa, Artificial Sequence
  peptide
CRDGPQGIAGQDRC

SEQ ID NO: 16 - protein, 5 aa, Artificial Sequence
  peptide
PHSRN

SEQ ID NO: 17 - protein, 9 aa, Artificial Sequence
  peptide
GGPQGIAGQ

SEQ ID NO: 18 - protein, 15 aa, Artificial Sequence
  peptide
GCREGPLGMRGERCG

SEQ ID NO: 19 - protein, 7 aa, Artificial Sequence
  peptide
LPRTGGK

SEQ ID NO: 20 - protein, 24 aa, Artificial Sequence
  peptide
PHSRNGGGKGGGERCGGGRGDSPY

SEQ ID NO: 21 - protein, 4 aa, Artificial Sequence
  peptide
RGDS

SEQ ID NO: 22 - protein, 8 aa, Artificial Sequence
  peptide
PHSRNRGD

SEQ ID NO: 23 - protein, 24 aa, Artificial Sequence
  peptide
PHSRNGGGKGGGERCGGGRDGSPY

SEQ ID NO: 24 - protein, 4 aa, Artificial Sequence
  peptide
DGGG

SEQ ID NO: 25 - protein, 5 aa, Artificial Sequence
  Peptide where Pro is a hydroxyproline
FPGER

SEQ ID NO: 26 - protein, 4 aa, Artificial Sequence
  Peptide
IAGQ

SEQ ID NO: 27 - protein, 9 aa, Artificial Sequence
  Peptide
IAGQLPRTG

SEQ ID NO: 28 - protein, 13 aa, Artificial Sequence
  Peptide
GPQGIAGQLPRTG

SEQ ID NO: 29 - protein, 13 aa, Artificial Sequence
  Peptide
LPRTGGPQGIAGQ

SEQ ID NO: 30 - protein, 11 aa, Artificial Sequence
  Peptide
GCRDLPRTGGG

SEQ ID NO: 31 - protein, 12 aa, Artificial Sequence
  Peptide
GPQGIWGQDRCG